(19) United States
(12) Patent Application Publication
(10) Pub. No.: US 2023/0279487 A1
(43) Pub. Date: Sep. 7, 2023

(54) METHODS, SYSTEMS, AND APPARATUS FOR HIGH THROUGHPUT SEQUENCING

(71) Applicant: Ultima Genomics, Inc., Newark, CA (US)

(72) Inventors: Gilad ALMOGY, Palo Alto, CA (US); Nathan BECKETT, Oakland, CA (US); Mark PRATT, Bozeman, MT (US); Anatoly A. SURDUTOVICH, Redwood City, CA (US); Nathan CASWELL, Sunnyvale, CA (US); Patrick D. KINNEY, Hayward, CA (US)

(21) Appl. No.: 18/124,481

(22) Filed: Mar. 21, 2023

Related U.S. Application Data
(63) Continuation of application No. PCT/US2021/052902, filed on Sep. 30, 2021.
(60) Provisional application No. 63/085,791, filed on Sep. 30, 2020.

(51) Int. Cl. C12Q 1/6869; B01L 3/00; G01N 35/10

(52) U.S. Cl. CPC C12Q 1/6869; B01L 3/5085; G01N 35/1002; B01L 2200/16; B01L 2300/023

(57) ABSTRACT

Provided herein methods, systems, and apparatus for high throughput sequencing, such as at an industrial scale. A sequencing system and/or apparatus may comprise one or more stations that can be operated in parallel and/or independent of one or more other stations.

### CROSS-REFERENCE

This application is a continuation of International Patent Application No. PCT/US2021/52902, filed Sep. 30, 2021, which claims benefit of U.S. Provisional Patent Application No. 63/085,791, filed Sep. 30, 2020, which is entirely incorporated herein by reference.

### BACKGROUND

Biological sample processing has various applications in the fields of molecular biology and medicine (e.g., diagnosis). For example, nucleic acid sequencing may provide information that may be used to diagnose a certain condition in a subject and in some cases tailor a treatment plan. Sequencing is widely used for molecular biology applications, including vector designs, gene therapy, vaccine design, industrial strain design and verification. Biological sample processing may involve a fluidics system and/or a detection system.

### SUMMARY

The advent of next generation sequencing (NGS) and massively parallel sequencing technologies have significantly increased the efficiency of sequencing compared to prior generation technologies (e.g., Sanger sequencing), such as by simultaneously processing clonally amplified nucleic acid templates in a flow cell. However, these technologies are not readily scalable and often require intimate operator attention and intervention. Significantly, for these sequencing processes, once a sequencer is started, the various process flows are inflexible and cannot be adjusted during a sequencing run or cycle until the sequencing run or cycle has finished. Recognized herein is a need for methods, systems, and apparatus that address at least the abovementioned problems.

Provided herein are methods, systems, and apparatus for high throughput sequencing, such as at an industrial scale. When applicable, embodiments disclosed hereinbelow can be performed, independently or in combination with other embodiments, in connection with any aspect of the systems, methods and/or apparatus disclosed herein.

In an aspect, provided herein is a method for sequencing a plurality of nucleic acid samples, the method comprising: (a) providing a nucleic acid sequencer having (i) a processing station configured to bring a nucleic acid molecule of a nucleic acid sample of the plurality of nucleic acid samples immobilized adjacent to a substrate into contact with a reagent to sequence the nucleic acid molecule; (ii) a sample station configured to supply the nucleic acid sample to the processing station; (iii) a substrate station configured to supply the substrate to the processing station, which substrate immobilizes adjacent thereto the nucleic acid sample; and (iv) a reagent station configured to supply the reagent to the processing station, wherein the reagent is supplied from a first reservoir or a second reservoir; (b) executing, by one or more processors individually or collectively, (i) at least a portion of a first queuing instruction to introduce a first set of one or more nucleic acid samples of the plurality of nucleic acid samples, including the nucleic acid sample, from the sample station to the processing station according to a first order of introduction defined by the first queuing instruction; (ii) a substrate loading instruction to introduce the substrate from the substrate station to the processing station and immobilize the first set of one or more nucleic acid samples adjacent to the substrate; and (iii) a sequencing instruction to draw the reagent from the first reservoir, from the second reservoir, or alternately from the first reservoir and the second reservoir and deliver the reagent to the processing station; and (c) while the processing station is in operation, performing one or more actions selected from the group consisting of: (1) introducing an additional nucleic acid sample to the sample station, (2) inputting a second queuing instruction and executing at least a portion of the second queuing instruction, wherein the second queuing instruction defines a second order of introduction that is different than the first order of introduction, (3) introducing an additional substrate to the substrate station, and (4) introducing an additional volume of the reagent to the reagent station by one or more of (i) replacing the first reservoir or the second reservoir with a third reservoir containing the reagent and (ii) replenishing the first reservoir or the second reservoir with the reagent.

In another aspect, provided is a system for sequencing a plurality of nucleic acid samples, comprising: a processing station configured to bring a nucleic acid molecule of a nucleic acid sample of the plurality of nucleic acid samples immobilized adjacent to a substrate into contact with a reagent to sequence the nucleic acid molecule; a sample station configured to supply the nucleic acid sample to the processing station; a substrate station configured to supply the substrate to the processing station, which substrate is configured to immobilize adjacent thereto the nucleic acid sample; a reagent station configured to supply the reagent to the processing station, wherein the reagent is supplied from a first reservoir or a second reservoir; and one or more processors, individually or collectively, programmed to execute (i) at least a portion of a first queuing instruction to introduce a first set of one or more nucleic acid samples of the plurality of nucleic acid samples, including the nucleic acid sample, from the sample station to the processing station according to a first order of introduction defined by the first queuing instruction, (ii) a substrate loading instruction to introduce the substrate from the substrate station to the processing station and immobilize the first set of one or more nucleic acid samples adjacent to the substrate, and (iii) a sequencing instruction to draw the reagent from the first reservoir, from the second reservoir, or alternately from the first reservoir and the second reservoir and deliver the reagent to the processing station, wherein the processing station is capable of operating during performance of one or more actions selected from the group consisting of: (1) introducing an additional nucleic acid sample of the plurality of nucleic acid samples to the sample station, (2) inputting a second queuing instruction and executing at least a portion of the second queuing instruction, wherein the second queuing instruction defines a second order of introduction that is different than the first order of introduction, (3) introducing an additional substrate to the substrate station, and (4) introducing an additional volume of the reagent to the reagent station by one or more (i) replacing the first reservoir or the second reservoir with a third reservoir containing the reagent and (ii) replenishing the first reservoir or the second reservoir with the reagent.

In some embodiments, the processing station is configured to operate for at least 24 hours without or with only minimum human intervention. In some embodiments, the processing station is configured to operate for at least 10 days without or with only minimum human intervention. Minimum human intervention refers to restocking or loading reagents or substrate while the processing station continuously operates to render sequence reads.

In some embodiments, (c) comprises performing two or more actions selected from the group consisting of (1), (2), (3), and (4). In some embodiments, (c) comprises performing three or more actions selected from the group consisting of (1), (2), (3), and (4). In some embodiments, (c) comprises performing each of (1), (2), (3), and (4).

In some embodiments, the sequencing instruction in (b)(iii) comprises instructions to draw the reagent from the first reservoir until the first reservoir is depleted below a predetermined threshold level, then to draw the reagent from the second reservoir.

In some embodiments, (4) comprises replacing or replenishing a reservoir from the first reservoir and the second reservoir that is depleted below a predetermined threshold level.

In some embodiments, the reagent comprises one or more members selected from the group consisting of a nucleotide solution, a cleaving solution, and a washing solution. In some embodiments, the nucleotide solution comprises one or more members selected from the group consisting of adenine-containing nucleotides, cytosine-containing nucleotides, guanine-containing nucleotides, thymine-containing nucleotides, and uracil-containing nucleotides. In some embodiments, the nucleotide solution comprises labeled nucleotides.

In some embodiments, the substrate is a wafer.

In some embodiments, the substrate comprises a substantially planar array. In some embodiments, the substrate comprises a plurality of independently addressable locations.

In some embodiments, the substrate is configured to rotate about an axis in the processing station. In some embodiments, the substrate is configured to linearly translate in the processing station.

In some embodiments, the nucleic acid molecule is coupled to a bead, wherein the bead is immobilized adjacent to the substrate.

In some embodiments, a plurality of nucleic acid samples is immobilized adjacent to the substrate, wherein nucleic acid samples of the plurality of nucleic acid samples are from different sources. In some embodiments, the plurality of nucleic acid samples is compatible with a common sequencing protocol.

In some embodiments, the processing station is disposed in a first environment different from a second environment in which the sample station, substrate station, and/or reagent station is disposed. In some embodiments, the first environment has a higher relative humidity than the second environment. In some embodiments, the first environment comprises one or more regions of controlled average temperature different from a second average temperature of the second environment.

In some embodiments, the processing station is disposed in an environment different from an ambient environment. In some embodiments, the environment has a higher relative humidity than the ambient environment. In some embodiments, the environment comprises one or more regions of controlled average temperature different from an ambient temperature.

In some embodiments, the nucleic acid sequencer comprises a dilution station configured to dilute the reagent from the reagent station prior to delivery of the reagent to the processing station. In some embodiments, the reagent is diluted with deionized water.

In some embodiments, the substrate station comprises a sealed environment. In some embodiments, the substrate station comprises a hermetically sealed environment. In some embodiments, the substrate station comprise a vacuum desiccator.

In some embodiments, the one or more processors are configured to, individually or collectively, within at most 40 hours of running time of the processing station, output one or more selected from the group consisting of: (i) at least 1.0 giga reads per substrate, (ii) sequence reads averaging at least 140 base pairs (bp) in length, and (iii) at least 0.2 terabase reads per run. In some embodiments, the one or more processors are configured to, within at most 40 hours of running time of the processing station, output at least 40.0 Giga reads per substrate. In some embodiments, the one or more processors are configured to, within at most 40 hours of running time of the processing station, output at least 500 bp read length. In some embodiments, the one or more processors are configured to, within at most 40 hours of running time of the processing station, output sequence reads including sequences of at least 6.5 tera nucleotide bases per run. As disclosed herein, 1 giga reads refer to 1 billion sequence reads and a ltera reads refer to 1 trillion sequence reads, etc.

In some embodiments, the one or more processors are configured to, within at most 25 hours of running time of the processing station, output one or more selected from the group consisting of: (i) at least 1.5 giga reads per substrate, (ii) at least 140 base pairs (bp) read length, and (iii) at least 0.2 terabase reads per run.

In some embodiments, the one or more processors are configured to, within at most 15 hours of running time of the processing station, output one or more selected from the group consisting of: (i) at least 1.5 giga reads per substrate, (ii) at least 140 base pairs (bp) read length, and (iii) at least 0.2 terabase reads per run.

In some embodiments, the method further comprises (A) inputting (1) the plurality of nucleic acid samples, including the nucleic acid sample, to the sample station and (2) a plurality of substrates, including the substrate, to the substrate station; and (B) providing to the one or more processors user instructions to start two or more sequencing cycles. In some embodiments, the method further comprises (C) in a first sequencing cycle, processing a first nucleic acid sample from the plurality of nucleic acid samples on a first substrate of the plurality of substrates; and (D) during or subsequent to the first sequencing cycle, in a second sequencing cycle, processing a second nucleic acid sample from the plurality of nucleic acid samples on a second substrate of the plurality of substrates, wherein the second sequencing cycle is performed in absence of additional user intervention. In some embodiments, the two or more sequencing cycles are at least 5 sequencing cycles. In some embodiments, the two or more sequencing cycles are at least 10 sequencing cycles. In some embodiments, the two or more sequencing cycles are at least 20 sequencing cycles.

In some embodiments, the method further comprises purifying a reagent mixture comprising the reagent prior to delivery of the reagent to the processing station, wherein the reagent mixture comprises a plurality of nucleotides or nucleotide analogs.

In some embodiments, the purifying comprises (A) directing the reagent mixture to a reaction space comprising a support having a first plurality of nucleic acid molecules immobilized adjacent thereto; (B) incorporating a subset of nucleotides or nucleotide analogs from the plurality of nucleotides or nucleotide analogs into the first plurality of nucleic acid molecules, thereby providing a remainder of the plurality of nucleotides or nucleotides analogs, wherein (B) is performed without detecting the subset of nucleotides incorporated into the plurality of nucleic acid molecules; and (C) delivering the remainder of the plurality of nucleotides or nucleotide analogs to the processing station. In some embodiments, the method further comprises (D) incorporating at least a subset of the remainder of the plurality of nucleotides or nucleotides analogs into a growing stand associated with the nucleic acid molecule.

In some embodiments, the subset of nucleotides or nucleotide analogs comprises less than 10%, less than 5%, less than 1%, less than 0.1%, or less than 0.01% of the plurality of nucleotides or nucleotide analogs.

In some embodiments, the remainder of the plurality of nucleotides or nucleotide analogs has a ratio of a number of nucleotides or nucleotide analogs of one or more but less than all canonical types to a number of nucleotides or nucleotide analogs of all other canonical types which is greater than 19:1. In some embodiments, the ratio is at least 29:1. In some embodiments, the ratio is at least 99:1. In some embodiments, the ratio is at least 999:1.

In some embodiments, the purifying comprises (A) selecting from a set of canonical types of nucleotides or nucleotides analogs a subset of canonical types of nucleotides or nucleotide analogs; (B) directing the reagent mixture to a reaction space comprising a support having a plurality of nucleic acid molecules immobilized thereto, wherein a percentage of nucleotides or nucleotide analogs corresponding to the subset relative to all other nucleotides or nucleotide analogs in the mixture is greater than 50%; and (C) incorporating nucleotides or nucleotide analogs from the mixture that do not correspond to the subset into the plurality of nucleic acid molecules such that the percentage is increased following the incorporating, wherein (A)-(C) are performed in absence of sequencing or sequence identification of the plurality of nucleic acid molecules.

In some embodiments, the purifying comprises (A) directing the reagent mixture to a reaction space comprising a support having a plurality of nucleic acid molecules immobilized thereto; (B) incorporating a subset of nucleotides or nucleotide analogs form the plurality of nucleotides or nucleotide analogs into the plurality of nucleic acid molecules, thereby providing a remainder of the plurality of nucleotides or nucleotides analogs, wherein (A)-(B) are performed in absence of sequencing or sequence identification of the plurality of nucleic acid molecules. In some embodiments, the method further comprises (C) using the remainder of the plurality of nucleotides or nucleotide analogs to perform nucleic acid sequencing by synthesis.

In some embodiments, the processing station is configured to detect one or more signals or change thereof from the nucleic acid sample. In some embodiments, the nucleic acid sequencer further comprises a detection station configured to detect one or more signals or change thereof from the nucleic acid sample. In some embodiments, in (c), the detection system is in operation to detect the one or more signals or change thereof. In some embodiments, the processing system comprises the detection station.

In some embodiments, the nucleic acid sequencer comprises a network of sensors in operative communication with the one or more processors, wherein the one or more processors are configured to, based on one or more signals received from the network of sensors, calibrate, adjust, or maintain a component or process of the processing station, the sample station, the substrate station, or the reagent station, wherein the network of sensors comprises one or more sensors selected from the group consisting of a temperature sensor, pressure sensor, humidity sensor, weight sensor, friction sensor, flow meter, motion sensor, optical sensor, pH sensor, audio sensor, and voltage, current, and/or resistive sensor.

In another aspect, provided is a method for processing analytes, comprising: (a) executing, by one or more processors individually or collectively, at least a portion of a first queuing instruction to introduce a first set of one or more sample analytes of a plurality of sample analytes from a sample station of a system into a processing station of the system according to a first order of introduction defined by the first queuing instruction, wherein the sample station comprises a plurality of sample sources, wherein each of the plurality of sample sources is accessible for introduction of sample analytes from the plurality of samples sources into the processing station by one or more actuators, and wherein the first queuing instruction defines the first order of introduction of the sample analytes between the plurality of sample sources; (b) receiving a second queuing instruction, wherein the second queuing instruction defines a second order of introduction different from the first order of introduction; and (c) executing, by the one or more processors individually or collectively, at least a portion of the second queuing instruction to introduce a second set of one or more sample analytes of the plurality of sample analytes from the sample station to the processing station according to the second order of introduction while the system is in operation.

In some embodiments, (c) is performed while the processing system is in operation.

In some embodiments, the executing in (c) is performed in absence of terminating the operation of the processing station.

In some embodiments, during the operation, the processing station is maintained at a different environment than an ambient environment. In some embodiments, during the operation, the processing station is maintained at a different environment than an environment of the sample station. In some embodiments, during the operation, the processing station is maintained at a different temperature than an ambient temperature. In some embodiments, during the operation, the processing station is maintained at a different humidity than an ambient humidity.

In some embodiments, the processing station is configured to direct a sample analyte from a sample source in the sample station onto a substrate in the processing station. In some embodiments, the substrate is capable of processing a plurality of samples. In some embodiments, a group of samples are selected according to a sample selection instruction based at least in part on use of area of the substrate. In some embodiments, a group of samples are selected such that the group of samples can be processed using a first set of conditions which differs from a second set of conditions at which the other samples are processed.

In some embodiments, the processing station is configured to direct a reagent to contact a sample analyte from a sample source in the sample station.

In some embodiments, the processing station is configured to detect a signal associated with a sample analyte from a sample source in the sample station.

In some embodiments, the method further comprises, prior to (b), providing a new sample source in the sample station while the system is in operation. In some embodiments, the new sample source is provided while the processing station is in operation.

In some embodiments, the plurality of sample analytes comprises a plurality of nucleic acid molecules.

In some embodiments, the processing station is configured to detect one or more signals or change thereof from the plurality of sample analytes. In some embodiments, the system further comprises a detection station configured to detect one or more signals or change thereof from the plurality of sample analytes. In some embodiments, in (c), the detection system is in operation to detect the one or more signals or change thereof. In some embodiments, the processing system comprises the detection station.

In another aspect, provided is a method for processing analytes, comprising: (a) providing a first reagent source and a second reagent source in a reagent station, wherein each of the first reagent source and the second reagent source (i) comprises a first reagent, and (ii) is accessible for introduction of the first reagent from the reagent station to a processing station by a controller, wherein the processing station is configured to facilitate one or more operations using the first reagent; (b) directing the first reagent from the first reagent source to the processing station; (c) directing the first reagent from the second reagent source to the processing station; (d) while the processing station is in operation and receiving the first reagent from the second reagent source, (i) replacing the first reagent source with a third reagent source comprising the first reagent, wherein the third reagent source is accessible for introduction of the first reagent from the reagent station to the processing station by the controller, or (ii) replenishing the first reagent source with an additional volume of the first reagent; and (e) directing the first reagent from (i) the third reagent source, or (ii) the additional volume of the first reagent in the first reagent source, to the processing station.

In some embodiments, the controller is configured to control one or more actuators.

In some embodiments, the controller is configured to control one or more valves in fluid communication with the first reagent source or the second reagent source.

In some embodiments, (c) is initiated when the first reagent source is depleted below a predetermined threshold level. In some embodiments, the predetermined threshold level is a fully depleted level.

In some embodiments, (e) is initiated when the second reagent source is depleted below a predetermined threshold level. In some embodiments, the predetermined threshold level is a fully depleted level.

In some embodiments, (i) the replacing or (ii) the replenishing in (d) is performed in absence of terminating the operation of the processing station.

In some embodiments, the method further comprises diluting the first reagent with a diluent subsequent to departure from the reagent station and prior to delivery to the processing station. In some embodiments, the diluent is deionized water. In some embodiments, the diluent is delivered from a diluent source comprising the diluent. In some embodiments, the diluent is produced within an enclosure comprising therein the reagent station and the processing station.

In some embodiments, the directing the first reagent from the second reagent source in (c) commences subsequent to a volume of the first reagent in the first reagent source decreasing below a predetermined threshold. In some embodiments, the directing in (e) commences subsequent to a volume of the first reagent in the second reagent source decreasing below a second predetermined threshold. In some embodiments, the predetermined threshold and the second predetermined threshold are the same.

In some embodiments, during the operation, the processing station is maintained at a different environment than an ambient environment. In some embodiments, during the operation, the processing station is maintained at a different environment than an environment of the reagent station. In some embodiments, during the operation, the processing station is maintained at a different temperature than an ambient temperature. In some embodiments, during the operation, the processing station is maintained at a different humidity than an ambient humidity.

In some embodiments, the processing station is configured to direct the reagent to contact an analyte in the processing station. In some embodiments, the processing station is configured to detect a signal associated with the analyte. In some embodiments, the analyte is a nucleic acid molecule.

In some embodiments, the first reagent source comprises a container.

In some embodiments, the first reagent comprises a nucleotide solution, a washing solution, or a cleavage solution. In some embodiments, the nucleotide solution comprises adenine-containing nucleotides, cytosine-containing nucleotides, guanine-containing nucleotides, thymine-containing nucleotides, or uracil-containing nucleotides. In some embodiments, the nucleotide solution comprises labeled nucleotides.

In some embodiments, the method further comprises preparing the first reagent of the third reagent source or the additional volume of the first reagent source from a frozen concentrate.

In some embodiments, the processing station is configured to operate for at least 24 hours without human intervention. In some embodiments, the processing station is configured to operate for at least 40 hours without human intervention.

In some embodiments, the processing station is configured to detect one or more signals or change thereof from the analytes. In some embodiments, the method further comprises providing a detection station configured to detect one or more signals or change thereof from the analytes. In some embodiments, in (d), the detection system is in operation to detect the one or more signals or change thereof. In some embodiments, the processing system comprises the detection station.

In another aspect, provided is a method for processing analytes, comprising: (a) providing a plurality of substrates in a substrate station, wherein each of the plurality of substrates is accessible for introduction of substrates from the substrate station into a processing station of a system by one or more actuators; (b) delivering, by one or more actuators, a first substrate of the plurality of substrates into the processing station; (c) in the processing station, performing a process involving an analyte immobilized adjacent to the first substrate; and (d) delivering, by the one or more actuators, a second substrate of the plurality of substrates into the processing station while the system is in operation.

In some embodiments, the delivering in (d) is performed while the processing station is performing the process.

In some embodiments, the delivering in (d) is performed in absence of terminating the process of the processing station.

In some embodiments, during the process, the processing station is maintained at a different environment than an ambient environment. In some embodiments, during the process, the processing station is maintained at a different environment than an environment of the substrate station. In some embodiments, during the process, the processing station is maintained at a different temperature than an ambient temperature. In some embodiments, during the process, the processing station is maintained at a different humidity than an ambient humidity.

In some embodiments, the processing station is configured to perform processes on two or more substrates simultaneously.

In some embodiments, the processing station is configured to deposit the analyte onto the first substrate.

In some embodiments, the processing station is configured to direct a reagent to contact the analyte immobilized adjacent to the first substrate. In some embodiments, the reagent comprises a nucleotide solution, a washing solution, or a cleavage solution. In some embodiments, the nucleotide solution comprises adenine-containing nucleotides, cytosine-containing nucleotides, guanine-containing nucleotides, thymine-containing nucleotides, or uracil-containing nucleotides. In some embodiments, the nucleotide solution comprises labeled nucleotides.

In some embodiments, the processing station is configured to detect a signal associated with the analyte. In some embodiments, the signal is a fluorescent signal.

In some embodiments, the analyte is a nucleic acid molecule.

In some embodiments, plurality of substrates is a plurality of wafers.

In some embodiments, the first substrate is substantially planar.

In some embodiments, the first substrate is not a flow cell.

In some embodiments, the first substrate is patterned or textured.

In some embodiments, the substrate station comprises a rack containing the plurality of substrates. In some embodiments, the rack is a vertical rack that contains the plurality of substrates in a substantially horizontal position. In some embodiments, the rack is a horizontal rack that contains the plurality of substrates in a substantially vertical position.

In some embodiments, the first substrate is delivered to a first location of the processing station and the second substrate is delivered to a second location of the processing station that is different than the first location. In some embodiments, the second location is disposed below the first location. In some embodiments, the second location is adjacent to the first location. In some embodiments, further comprising removing the first substrate from the first location of the processing station. In some embodiments, further comprising delivering the second substrate to the first location of the processing station.

In some embodiments, the processing station is configured to operate, without human intervention, for at least 12 hours, 24 hours, 40 hours, 60 hours, 80 hours, 100 hours, 150 hours, 200 hours, 300 hours, 400 hours, 500 hours, 750 hours or 1000 hours. In some embodiments, minimum human intervention is needed, e.g., for replacing concentrated reagent stocks, loading additional substrate in a substrate holder, preferably while the processing station continuously operates to output sequence reads with no or minimum interruption. In some embodiments, the processing station is configured to detect one or more signals or change thereof from the analytes. In some embodiments, the system further comprises a detection station configured to detect one or more signals or change thereof from the analytes. In some embodiments, in (d), the detection system is in operation to detect the one or more signals or change thereof. In some embodiments, the processing system comprises the detection station.

In another aspect, provided is a method for processing analytes, comprising: (a) inputting (1) a plurality of nucleic acid samples from different sample sources, and (2) a plurality of substrates; (b) providing, to one or more processors, user instructions to start two or more sequencing cycles; (c) in a first sequencing cycle, processing a first nucleic acid sample of the plurality of nucleic acid samples on a first substrate of the plurality of substrates; and (d) during or subsequent to the first sequencing cycle, in a second sequencing cycle, processing a second nucleic acid sample of the plurality of nucleic acid samples on a second substrate of the plurality of substrates, wherein the second sequencing cycle is performed in absence of additional user intervention.

In some embodiments, the method further comprises, during or subsequent to an (n−1)^{th} sequencing cycle, in an nth sequencing cycle, processing an nth nucleic acid sample from the plurality of nucleic acid samples on an nth substrate of the plurality of substrates, wherein the nth sequencing cycle is performed in absence of additional user instructions from the user instructions.

In some embodiments, the plurality of substrates is a plurality of wafers.

In some embodiments, first substrate or the second substrate is substantially planar.

In some embodiments, the first substrate and the second substrate are not flow cells.

In some embodiments, the first substrate or the second substrate is textured or patterned.

In some embodiments, the first nucleic acid sample comprises a first plurality of nucleic acid molecules and the second nucleic acid sample comprises a second plurality of nucleic acid molecules.

In some embodiments, the method further comprises depositing the first nucleic acid sample onto the first substrate and depositing the second nucleic acid sample onto the second substrate.

In some embodiments, the first nucleic acid sample is immobilized adjacent to the first substrate and the second nucleic acid sample is immobilized adjacent to the second substrate. In some embodiments, the first nucleic acid sample is immobilized to the first substrate via a first plurality of particles and the second nucleic acid sample is immobilized to the second substrate via a second plurality of particles.

In some embodiments, the first sequencing cycle comprises directing, in sequence, a first set of reagents, a second set of reagents, a third set of reagents, and a fourth set of reagents to the first nucleic acid sample. In some embodiments, each of the first set of reagents, the second set of reagents, the third set of reagents, and the fourth set of reagents comprises a washing solution. In some embodiments, each of the first set of reagents, the second set of reagents, the third set of reagents, and the fourth set of reagents comprises a nucleotide solution. In some embodiments, the nucleotide solutions of the first set of reagents, the second set of reagents, the third set of reagents, and the fourth set of reagents comprise nucleotides of different canonical types. In some embodiments, the nucleotide solutions comprise labeled nucleotides. In some embodiments, each of the first set of reagents, the second set of reagents, the third set of reagents, and the fourth set of reagents comprises a cleavage solution.

In some embodiments, the first sequencing cycle comprises detecting signal associated with the first nucleic acid sample and the second sequencing cycle comprises detecting signal associated with the second nucleic acid sample. In some embodiments, the signal is fluorescent signal.

In another aspect, provided is a system, comprising: a sample station comprising a plurality of sample sources comprising a plurality of sample analytes, wherein the plurality of sample analytes comprises a first set of one or more sample analytes, wherein each of the plurality of sample sources is accessible for introduction of sample analytes from the plurality of sample sources into the processing station by one or more actuators; a processing station configured to receive sample analytes of the plurality of sample analytes; and one or more processors, individually or collectively, programmed to: (1) execute at least a portion of a first queuing instruction to introduce the first set of one or more sample analytes from the sample station into the processing station according to a first order of introduction defined by the first queuing instruction, wherein the first queuing instruction defines the first order of introduction of the sample analytes between the plurality of sample sources; (2) receive a second queuing instruction, wherein the second queuing instruction defines a second order of introduction different from the first order of introduction; and (3) execute at least a portion of the second queuing instruction to introduce a second set of one or more sample analytes of the plurality of sample analytes from the sample station to the processing station according to the second order of introduction while the system is in operation.

In some embodiments, the one or more processors are individually or collectively programmed to execute the at least the portion of the second queuing instruction while the processing station is in operation.

In some embodiments, (3) is performed in absence of terminating the operation of the processing station.

In some embodiments, during the operation, the processing station is maintained at (i) a different environment than an ambient environment, (ii) a different environment than an environment of the sample station, (iii) a different temperature than an ambient temperature, and/or (iv) a different humidity than an ambient humidity.

In some embodiments, the processing station is configured to direct a sample analyte from a sample source in the sample station onto a substrate in the processing station. In some embodiments, the substrate is capable of processing a plurality of samples. In some embodiments, a group of samples are selected according to a sample selection instruction based at least in part on use of area of the substrate. In some embodiments, a group of samples are selected such that the group of samples can be processed using a first set of conditions which differs from a second set of conditions at which the other samples are processed.

In some embodiments, the processing station is configured to direct a reagent to contact a sample analyte from a sample source in the sample station.

In some embodiments, the processing station is configured to detect a signal associated with a sample analyte from a sample source in the sample station.

In some embodiments, the processors are individually or collectively programmed to provide a new sample source in the sample station while the system is in operation.

In some embodiments, the plurality of sample analytes comprises a plurality of nucleic acid molecules.

In another aspect, provided is a system comprising: a reagent station comprising a first reagent source and a second reagent source, wherein each of the first reagent source and the second reagent source (i) comprises a first reagent and (ii) is accessible for introduction of the first reagent from the reagent station to a processing station by a controller; the processing station, wherein the processing station is configured to facilitate one or more operations using the first reagent; and one or more processors, individually or collectively, programmed to: (1) direct the first reagent from the first reagent source to the processing station; (2) direct the first reagent from the second reagent source to the processing station; (3) while the processing station is in operation and receiving the first reagent from the second reagent source, (i) replace the first reagent source with a third reagent source comprising the first reagent, wherein the third reagent source is accessible for introduction of the first reagent from the reagent station to the processing station by the controller, or (ii) replenish the first reagent source with an additional volume of the first reagent; and (4) direct the first reagent from (i) the third reagent source, or (ii) the additional volume of the first reagent in the first reagent source, to the processing station.

In another aspect, provided is a system comprising: a substrate station comprising a plurality of substrates, wherein each of the plurality of substrates is accessible for introduction of substrates from the substrate station into a processing station by one or more actuators; the processing station; and one or more processors, individually or collectively, programmed to: (1) deliver, by one or more actuators, a first substrate of the plurality of substrates into the processing station; (2) in the processing station, perform a process involving an analyte immobilized adjacent to the first substrate; and (3) deliver, by the one or more actuators, a second substrate of the plurality of substrates into the processing station while the system is in operation.

In some embodiments, the one or more processors are individually or collectively programmed to deliver the second substrate while the processing station is performing the process.

In another aspect, provided is a system comprising: a processing station configured to receive nucleic acid samples of a plurality of nucleic acid samples from different sample sources and substrates of a plurality of substrates; and one or more processors, individually or collectively, programmed to: (1) provide a first nucleic acid sample of the plurality of nucleic acid samples to a first substrate of the plurality of substrates; (2) provide a second nucleic acid sample of the plurality of nucleic acid samples to a second substrate of the plurality of substrates; (3) receive user instructions to start two or more sequencing cycles; (4) initiate a first sequencing cycle to process the first nucleic acid sample; and (5) during or subsequent to the first sequencing cycle, initiate a second sequencing cycle to process the second nucleic acid sample, wherein the second sequencing cycle is configured to be performed in absence of additional user intervention.

In some embodiments, the one or more processors are individually or collectively programmed to, during or subsequent to an (n−1)^{th} sequencing cycle, initiate an nth sequencing cycle to process an nth nucleic acid sample of the plurality of nucleic acid samples on an nth substrate of the plurality of substrates, wherein the nth sequencing cycle is configured to be performed in absence of additional user instructions from the user instructions.

It will be understood that embodiments disclosing various configuration and capacity of the one or more processers and/or processing station can be performed, individually or in combination with other embodiments, in connection with any aspect of the systems, methods and/or apparatus disclosed throughout.

In some embodiments, the one or more processors are configured to output one or more selected from the group consisting of: (i) at least 1.0 giga reads per substrate, (ii) sequence reads averaging at least 140 base pairs (bp) in length, and (iii) at least 0.2 terabase reads per run. In some embodiments, the one or more processors are configured to output at least 1.0 Giga reads, at least 1.5 Giga reads, at least 2.0 Giga reads, at least 6.0 giga reads, at least 10.0 giga reads, at least 20.0 giga reads, at least 40.0 giga reads, at least 50.0 giga reads, at least 100.0 giga reads, at least 200.0 giga reads, at least 500.0 giga reads, or at least 1 tera reads, per substrate. In some embodiments fewer than 1.0 giga reads can be generated for applications requiring a fast turnaround and less data, including but not limited to quick diagnostic tests for pathogens.

In some embodiments, the one or more processors are configured to output sequence reads of an average length up to about 100 bp, up to about 150 bp, up to about 200 bp, up to about 250 bp, up to about 300 bp, up to about 400 bp or longer, or up to about 500 bp. In some embodiments, the one or more processors are configured to output sequence reads of an average length longer than 500 bp, such as up to 550 bp, 600 bp, 700 bp, 800 bp, 900 bp, or up to 1000 bp or longer.

In some embodiments, the one or more processors are configured to output sequence reads comprising a total of up to about 0.4 tera bases, up to around 1 tera bases, up to around 1.5 tera bases, up to around 6.0 tera bases, up to around 6.5 tera bases, up to around 10 tera bases, up to around 20 tera bases, up to around 50 tera bases, up to around 100 tera bases, up to around 200 tera bases, up to around 300 tera bases, up to around 500 tera bases, up to around 1 peta bases, per run.

In some embodiments, the one or more processors are configured to output sequence information, continuously or with minimum human intervention, at a rate of up to 5 bp per hour (5 bp/hr: read out of 5 nucleotide sequence on each sequence read per hour), up to 10 bp/hr, up to 15 bp/hr, up to 20 bp/hr, up to 25 bp/hr, up to 30 bp/hr, up to 35 bp/hr, up to 40 bp/hr, up to 50 bp/hr, up to 60 bp/hr, up to 70 bp/hr, up to 80 bp/hr, up to 90 bp/hr, up to 100 bp/hr, up to 120 bp/hr, up to 150 bp/hr, up to 200 bp/hr, up to 250 bp/hr, up to 300 bp/hr, up to 400 bp/hr, or up to 100 bp/hr. In some embodiments, sequence information may be generated at fewer than 5 bp/hr or more than 500 bp/hr.

In some embodiments, the one or more processors are configured to output one or more selected from the group consisting of: (i) at least 1.0 giga reads per substrate, (ii) sequence reads averaging at least 140 base pairs (bp) in length, and (iii) at least 0.2 tera bases of sequence per run. As disclosed herein, a sequence run can be at most 5 hours, at most 10 hours, at most 15 hours, at most 20 hours, at most 25 hours, at most 30 hours, at most 35 hours, or at most 40 hours, of continuous running time of the processing station.

In some embodiments, the processing station is configured to detect one or more signals or change thereof from the plurality of nucleic acid samples. In some embodiments, the system further comprises a detection station configured to detect one or more signals or change thereof from the plurality of nucleic acid samples. In some embodiments, the processing system comprises the detection station.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein) of which:

FIG. 1 illustrates a non-limiting example of a high throughput system, as described herein.

FIG. 2 illustrates non-limiting examples of arrays on a substrate, as described herein.

FIGS. 3A-3C illustrate non-limiting examples of different stations in a sequencing system, as described herein.

FIGS. 3D-3E illustrate non-limiting example components of sample environment systems, as described herein.

FIG. 3F illustrates non-limiting example components of detection systems, as described herein.

FIG. 4 shows a computer system that is programmed or otherwise configured to implement methods provided herein.

FIG. 5 shows a non-limiting example of a system for sequencing a nucleic acid molecule, as described herein.

FIG. 6 shows a non-limiting example of a bowl implementation of the modular sample environment, as described herein.

FIGS. 7A and 7B illustrate non-limiting examples of cross-sectional views of the bowl and inner compartments, as described herein.

FIGS. 8A and 8B illustrate a non-limiting example of a liquid catching structure, as described herein.

FIG. 9 illustrates a non-limiting example of a cross-section of the bowl modular sample environment during a cleaning cycle, as described herein.

FIGS. 10A and 10B illustrate a non-limiting example of a bowl in fluid communication with a drain assembly and further downstream fluidic systems, as described herein.

FIG. 11 shows a non-limiting example of a heating and/or cooling element in contact with a bowl, as described herein.

FIG. 12 shows a flow diagram for a method of cleaning the chemical ceiling, as described herein.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Where values are described as ranges, it will be understood that such disclosure includes the disclosure of all possible sub-ranges within such ranges, as well as specific numerical values that fall within such ranges irrespective of whether a specific numerical value or specific sub-range is expressly stated.

The terms “about” and “approximately” shall generally mean an acceptable degree of error or variation for a given value or range of values, such as, for example, a degree of error or variation that is within 20 percent (%), within 15%, within 10%, or within 5% of a given value or range of values.

The term “subject,” as used herein, generally refers to an individual or entity from which a biological sample (e.g., a biological sample that is undergoing or will undergo processing or analysis) may be derived. A subject may be an animal (e.g., mammal or non-mammal) or plant. The subject may be a human, dog, cat, horse, pig, bird, non-human primate, simian, farm animal, companion animal, sport animal, or rodent. A subject may be a patient. The subject may have or be suspected of having a disease or disorder, such as cancer (e.g., breast cancer, colorectal cancer, brain cancer, leukemia, lung cancer, skin cancer, liver cancer, pancreatic cancer, lymphoma, esophageal cancer or cervical cancer) or an infectious disease. Alternatively or additionally, a subject may be known to have previously had a disease or disorder. The subject may have or be suspected of having a genetic disorder such as achondroplasia, alpha-1 antitrypsin deficiency, antiphospholipid syndrome, autism, autosomal dominant polycystic kidney disease, Charcot-Marie-tooth, cri du chat, Crohn's disease, cystic fibrosis, Dercum disease, down syndrome, Duane syndrome, Duchenne muscular dystrophy, factor V Leiden thrombophilia, familial hypercholesterolemia, familial Mediterranean fever, fragile x syndrome, Gaucher disease, hemochromatosis, hemophilia, holoprosencephaly, Huntington's disease, Klinefelter syndrome, Marfan syndrome, myotonic dystrophy, neurofibromatosis, Noonan syndrome, osteogenesis imperfecta, Parkinson's disease, phenylketonuria, Poland anomaly, porphyria, progeria, retinitis pigmentosa, severe combined immunodeficiency, sickle cell disease, spinal muscular atrophy, Tay-Sachs, thalassemia, trimethylaminuria, Turner syndrome, velocardiofacial syndrome, WAGR syndrome, or Wilson disease. A subject may be undergoing treatment for a disease or disorder. A subject may be symptomatic or asymptomatic of a given disease or disorder. A subject may be healthy (e.g., not suspected of having disease or disorder). A subject may have one or more risk factors for a given disease. A subject may be under the care of one or more health professionals. A subject may have a given weight, height, body mass index, or other physical characteristics. A subject may have a given ethnic or racial heritage, place of birth or residence, nationality, disease or remission state, family medical history, or other characteristic.

The term “sample,” as used herein, generally refers to a biological sample. The systems, methods, and apparatus provided herein may be particularly beneficial for analyzing biological samples, which can be highly sensitive to the environment, such as to the temperature, pressure, and/or humidity of the environment. Biological samples may be derived from any subject or living organism. For example, a subject may be an animal, a mammal, an avian, a vertebrate, a rodent (e.g., a mouse), a primate, a simian, a human, or other organism, such as a plant (e.g., as described herein). Animals may include, but are not limited to, farm animals, sport animals, and pets. A subject can be a healthy or asymptomatic individual, an individual that has or is suspected of having a disease (e.g., cancer) or a pre-disposition to the disease, and/or an individual that is in need of therapy or suspected of needing therapy. A subject can be a patient. A subject can be a microorganism or microbe (e.g., bacteria, fungi, archaea, viruses).

A sample may be obtained from a subject. The biological sample may be obtained directly or indirectly from the subject. A sample may be obtained from a subject via any suitable method, including, but not limited to, spitting, swabbing, blood draw, biopsy, obtaining excretions (e.g., urine, stool, sputum, vomit, or saliva), excision, scraping, and puncture. A sample may be obtained from a subject by, for example, intravenously or intraarterially accessing the circulatory system, collecting a secreted biological sample (e.g., stool, urine, saliva, sputum, etc.), breathing, or surgically extracting a tissue (e.g., biopsy). The sample may be obtained by non-invasive methods including but not limited to: scraping of the skin or cervix, swabbing of the cheek, or collection of saliva, urine, feces, menses, tears, or semen. Alternatively, the sample may be obtained by an invasive procedure such as biopsy, needle aspiration, or phlebotomy. A sample may comprise a bodily fluid such as, but not limited to, blood (e.g., whole blood, red blood cells, leukocytes or white blood cells, platelets), plasma, serum, sweat, tears, saliva, sputum, urine, semen, mucus, synovial fluid, breast milk, colostrum, amniotic fluid, bile, bone marrow, interstitial or extracellular fluid, or cerebrospinal fluid. For example, a sample may be obtained by a puncture method to obtain a bodily fluid comprising blood and/or plasma. Such a sample may comprise both cells and cell-free nucleic acid material. Alternatively, the sample may be obtained from any other source including but not limited to blood, sweat, hair follicle, buccal tissue, tears, menses, feces, or saliva. The biological sample may be a tissue sample, such as a tumor biopsy. The sample may be obtained from any of the tissues provided herein including, but not limited to, skin, heart, lung, kidney, breast, pancreas, liver, intestine, brain, prostate, esophagus, muscle, smooth muscle, bladder, gall bladder, colon, or thyroid. The methods of obtaining provided herein include methods of biopsy including fine needle aspiration, core needle biopsy, vacuum assisted biopsy, large core biopsy, incisional biopsy, excisional biopsy, punch biopsy, shave biopsy or skin biopsy. The sample may be a tissue sample, such as a biopsy, core biopsy, needle aspirate, or fine needle aspirate. The sample may be a fluid sample, such as a blood sample, urine sample, or saliva sample. The sample may be a skin sample. The sample may be a cheek swab. The sample may be a plasma or serum sample.

The biological sample may comprise one or more cells. The biological sample may be a cell line or cell culture sample. A biological sample may comprise one or more nucleic acid molecules such as one or more deoxyribonucleic acid (DNA) and/or ribonucleic acid (RNA) molecules (e.g., included within cells or not included within cells). Nucleic acid molecules may be included within cells. Alternatively or additionally, nucleic acid molecules may not be included within cells (e.g., cell-free nucleic acid molecules). The biological sample may be a cell-free sample. A cell-free sample may include extracellular polynucleotides. Extracellular polynucleotides may be isolated from a bodily sample that may be selected from the group consisting of blood, plasma, serum, urine, saliva, mucosal excretions, sputum, stool and tears. The biological sample can include one or more microbes. The biological sample may be a nucleic acid sample or protein sample. The biological sample may also be a carbohydrate sample or a lipid sample. The biological sample may be derived from another sample.

A biological sample may comprise one or more biological particles. The biological particle may be a macromolecule. The biological particle may be a small molecule. The biological particle may be a virus. The biological particle may be a cell or derivative of a cell. The biological particle may be an organelle. The biological particle may be a rare cell from a population of cells. The biological particle may be any type of cell, including without limitation prokaryotic cells, eukaryotic cells, bacterial, fungal, plant, mammalian, or other animal cell type, mycoplasmas, normal tissue cells, tumor cells, or any other cell type, whether derived from single cell or multicellular organisms. The biological particle may be a constituent (e.g., macromolecular constituent) of a cell, such as deoxyribonucleic acids (DNA), ribonucleic acids (RNA), nucleus, organelles, proteins, peptides, polypeptides, or any combination thereof. The RNA may be coding or non-coding. The RNA may be messenger RNA (mRNA), ribosomal RNA (rRNA) or transfer RNA (tRNA), for example. The RNA may be a transcript. The RNA may be small RNA that are less than 200 nucleic acid bases in length, or large RNA that are greater than 200 nucleic acid bases in length. Small RNAs may include 5.8S ribosomal RNA (rRNA), 5S rRNA, transfer RNA (tRNA), microRNA (miRNA), small interfering RNA (siRNA), small nucleolar RNA (snoRNAs), Piwi-interacting RNA (piRNA), tRNA-derived small RNA (tsRNA) and small rDNA-derived RNA (srRNA). The RNA may be double-stranded RNA or single-stranded RNA. The RNA may be circular RNA. The biological particle may be a hardened cell. Such hardened cell may or may not include a cell wall or cell membrane. Alternatively or additionally, samples of the present disclosure may include non-biological samples.

The term “cell-free sample,” as used herein, generally refers to a sample that is substantially free of cells (e.g., less than 10% cells on a volume basis). A cell-free sample may be derived from any source (e.g., as described herein). For example, a cell-free sample may be derived from blood, sweat, urine, or saliva. For example, a cell-free sample may be derived from a tissue or bodily fluid. A cell-free sample may be derived from a plurality of tissues or bodily fluids. For example, a sample from a first tissue or fluid may be combined with a sample from a second tissue or fluid (e.g., while the samples are obtained or after the samples are obtained). In an example, a first fluid and a second fluid may be collected from a subject (e.g., at the same or different times) and the first and second fluids may be combined to provide a sample. A cell-free sample may comprise one or more nucleic acid molecules such as one or more DNA or RNA molecules.

A sample that is not a cell-free sample (e.g., a sample comprising one or more cells) may be processed to provide a cell-free sample. For example, a sample that includes one or more cells as well as one or more nucleic acid molecules (e.g., DNA and/or RNA molecules) not included within cells (e.g., cell-free nucleic acid molecules) may be obtained from a subject. The sample may be subjected to processing (e.g., as described herein) to separate cells and other materials from the nucleic acid molecules not included within cells, thereby providing a cell-free sample (e.g., comprising nucleic acid molecules not included within cells). The cell-free sample may then be subjected to further analysis and processing (e.g., as provided herein). Nucleic acid molecules not included within cells (e.g., cell-free nucleic acid molecules) may be derived from cells and tissues. For example, cell-free nucleic acid molecules may derive from a tumor tissue or a degraded cell (e.g., of a tissue of a body). Cell-free nucleic acid molecules may comprise any type of nucleic acid molecules (e.g., as described herein). Cell-free nucleic acid molecules may be double-stranded, single-stranded, or a combination thereof. Cell-free nucleic acid molecules may be released into a bodily fluid through secretion or cell death processes, e.g., cellular necrosis, apoptosis, or the like. Cell-free nucleic acid molecules may be released into bodily fluids from cancer cells (e.g., circulating tumor DNA (ctDNA)). Cell free nucleic acid molecules may also be fetal DNA circulating freely in a maternal blood stream (e.g., cell-free fetal nucleic acid molecules such as cffDNA). Alternatively or additionally, cell-free nucleic acid molecules may be released into bodily fluids from healthy cells.

A biological sample may be obtained directly from a subject and analyzed without any intervening processing, such as, for example, sample purification or extraction. For example, a blood sample may be obtained directly from a subject by accessing the subject's circulatory system, removing the blood from the subject (e.g., via a needle), and transferring the removed blood into a receptacle. The receptacle may comprise reagents (e.g., anti-coagulants) such that the blood sample is useful for further analysis. Such reagents may be used to process the sample or analytes derived from the sample in the receptacle or another receptacle prior to analysis. In another example, a swab may be used to access epithelial cells on an oropharyngeal surface of the subject. Following obtaining the biological sample from the subject, the swab containing the biological sample may be contacted with a fluid (e.g., a buffer) to collect the biological fluid from the swab.

Any suitable biological sample that comprises one or more nucleic acid molecules may be obtained from a subject. A sample (e.g., a biological sample or cell-free biological sample) suitable for use according to the methods provided herein may be any material comprising tissues, cells, degraded cells, nucleic acids, genes, gene fragments, expression products, gene expression products, and/or gene expression product fragments of an individual to be tested. A biological sample may be solid matter (e.g., biological tissue) or may be a fluid (e.g., a biological fluid). In general, a biological fluid may include any fluid associated with living organisms. Non-limiting examples of a biological sample include blood (or components of blood—e.g., white blood cells, red blood cells, platelets) obtained from any anatomical location (e.g., tissue, circulatory system, bone marrow) of a subject, cells obtained from any anatomical location of a subject, skin, heart, lung, kidney, breath, bone marrow, stool, semen, vaginal fluid, interstitial fluids derived from tumorous tissue, breast, pancreas, cerebral spinal fluid, tissue, throat swab, biopsy, placental fluid, amniotic fluid, liver, muscle, smooth muscle, bladder, gall bladder, colon, intestine, brain, cavity fluids, sputum, pus, microbiota, meconium, breast milk, prostate, esophagus, thyroid, serum, saliva, urine, gastric and digestive fluid, tears, ocular fluids, sweat, mucus, earwax, oil, glandular secretions, spinal fluid, hair, fingernails, skin cells, plasma, nasal swab or nasopharyngeal wash, spinal fluid, cord blood, emphatic fluids, and/or other excretions or body tissues. Methods for determining sample suitability and/or adequacy are provided. A sample may include, but is not limited to, blood, plasma, tissue, cells, degraded cells, cell-free nucleic acid molecules, and/or biological material from cells or derived from cells of an individual such as cell-free nucleic acid molecules. The sample may be a heterogeneous or homogeneous population of cells, tissues, or cell-free biological material. The biological sample may be obtained using any method that can provide a sample suitable for the analytical methods described herein.

A sample (e.g., a biological sample or cell-free biological sample) may undergo one or more processes in preparation for analysis, including, but not limited to, filtration, centrifugation, selective precipitation, permeabilization, isolation, agitation, heating, purification, and/or other processes. For example, a sample may be filtered to remove contaminants or other materials. In an example, a sample comprising cells may be processed to separate the cells from other material in the sample. Such a process may be used to prepare a sample comprising only cell-free nucleic acid molecules. Such a process may consist of a multi-step centrifugation process. Multiple samples, such as multiple samples from the same subject (e.g., obtained in the same or different manners from the same or different bodily locations, and/or obtained at the same or different times (e.g., seconds, minutes, hours, days, weeks, months, or years apart)) or multiple samples from different subjects may be obtained for analysis as described herein. In an example, the first sample is obtained from a subject before the subject undergoes a treatment regimen or procedure and the second sample is obtained from the subject after the subject undergoes the treatment regimen or procedure. Alternatively or additionally, multiple samples may be obtained from the same subject at the same or approximately the same time. Different samples obtained from the same subject may be obtained in the same or different manner. For example, a first sample may be obtained via a biopsy and a second sample may be obtained via a blood draw. Samples obtained in different manners may be obtained by different medical professionals, using different techniques, at different times, and/or at different locations. Different samples obtained from the same subject may be obtained from different areas of a body. For example, a first sample may be obtained from a first area of a body (e.g., a first tissue) and a second sample may be obtained from a second area of the body (e.g., a second tissue).

A biological sample as used herein (e.g., a biological sample comprising one or more nucleic acid molecules) may not be purified when provided in a reaction vessel. Furthermore, for a biological sample comprising one or more nucleic acid molecules, the one or more nucleic acid molecules may not be extracted when the biological sample is provided to a reaction vessel. For example, ribonucleic acid (RNA) and/or deoxyribonucleic acid (DNA) molecules of a biological sample may not be extracted from the biological sample when providing the biological sample to a reaction vessel. Moreover, a target nucleic acid (e.g., a target RNA or target DNA molecules) present in a biological sample may not be concentrated when providing the biological sample to a reaction vessel. Alternatively, a biological sample may be purified and/or nucleic acid molecules may be isolated from other materials in the biological sample.

A biological sample as described herein may contain a target nucleic acid. As used herein, the terms “template nucleic acid”, “target nucleic acid”, “nucleic acid molecule,” “nucleic acid sequence,” “nucleic acid fragment,” “oligonucleotide,” “polynucleotide,” and “nucleic acid” generally refer to polymeric forms of nucleotides of any length, such as deoxyribonucleotides (dNTPs) or ribonucleotides (rNTPs), or analogs thereof, and may be used interchangeably. Nucleic acids may have any three-dimensional structure, and may perform any function, known or unknown. A nucleic acid molecule may have a length of at least about 10 nucleic acid bases (“bases”), 20 bases, 30 bases, 40 bases, 50 bases, 100 bases, 200 bases, 300 bases, 400 bases, 500 bases, 1 kilobase (kb), 2 kb, 3, kb, 4 kb, 5 kb, 10 kb, 50 kb, or more. An oligonucleotide is typically composed of a specific sequence of four nucleotide bases: adenine (A); cytosine (C); guanine (G); and thymine (T) (uracil (U) for thymine (T) when the polynucleotide is RNA). Oligonucleotides may include one or more nonstandard nucleotide(s), nucleotide analog(s) and/or modified nucleotides. Non-limiting examples of nucleic acids include DNA, RNA, genomic DNA (e.g., gDNA such as sheared gDNA), cell-free DNA (e.g., cfDNA), synthetic DNA/RNA, coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, complementary DNA (cDNA), recombinant nucleic acids, branched nucleic acids, plasmids, vectors, isolated DNA of any sequence, isolated RNA of any sequence, nucleic acid probes, and primers. A nucleic acid may comprise one or more modified nucleotides, such as methylated nucleotides and nucleotide analogs. If present, modifications to the nucleotide structure may be made before or following assembly of the nucleic acid. The sequence of nucleotides of a nucleic acid may be interrupted by non-nucleotide components. A nucleic acid may be further modified following polymerization, such as by conjugation or binding with a reporter agent.

A target nucleic acid or sample nucleic acid as described herein may be amplified to generate an amplified product. A target nucleic acid may be a target RNA or a target DNA. When the target nucleic acid is a target RNA, the target RNA may be any type of RNA, including types of RNA described elsewhere herein. The target RNA may be viral RNA and/or tumor RNA. A viral RNA may be pathogenic to a subject. Non-limiting examples of pathogenic viral RNA include human immunodeficiency virus I (HIV I), human immunodeficiency virus n (HIV 11), orthomyxoviruses, Ebola virus, Dengue virus, influenza viruses (e.g., H1N1, H3N2, H7N9, or H5N1), herpesvirus, hepatitis A virus, hepatitis B virus, hepatitis C virus (e.g., armored RNA-HCV virus), hepatitis D virus, hepatitis E virus, hepatitis G virus, coronaviruses, Epstein-Barr virus, mononucleosis virus, cytomegalovirus, SARS virus, West Nile Fever virus, polio virus, and measles virus.

A biological sample may comprise a plurality of target nucleic acid molecules. For example, a biological sample may comprise a plurality of target nucleic acid molecules from a single subject. In another example, a biological sample may comprise a first target nucleic acid molecule from a first subject and a second target nucleic acid molecule from a second subject.

The term “analyte,” as used herein, generally refers to an object that is analyzed, one or more properties measured determined, or otherwise assayed. An analyte may be a biological analyte, that is, or derived from, a biological sample for example. An analyte may be a non-biological analyte, that is, or derived from, a non-biological sample for example.

The term “nucleotide,” as used herein, generally refers to a substance including a base (e.g., a nucleobase), sugar moiety, and phosphate moiety. A nucleotide may comprise a free base with attached phosphate groups. A substance including a base with three attached phosphate groups may be referred to as a nucleoside triphosphate. When a nucleotide is being added to a growing nucleic acid molecule strand, the formation of a phosphodiester bond between the proximal phosphate of the nucleotide to the growing chain may be accompanied by hydrolysis of a high-energy phosphate bond with release of the two distal phosphates as a pyrophosphate. The nucleotide may be naturally occurring or non-naturally occurring (e.g., a modified or engineered nucleotide). The nucleotide analog may be a modified, synthesized or engineered nucleotide. The nucleotide analog may not be naturally occurring or may include a non-canonical base. The naturally occurring nucleotide may include a canonical base. The nucleotide analog may include a modified polyphosphate chain (e.g., triphosphate coupled to a fluorophore). The nucleotide analog may comprise a label. The nucleotide analog may be terminated (e.g., reversibly terminated). The nucleotide analog may comprise an alternative base.

The term “nucleotide analog,” as used herein, may include, but is not limited to, a nucleotide that may or may not be a naturally occurring nucleotide. For example, a nucleotide analog may be derived from and/or include structural similarities to a canonical nucleotide such as adenine-(A), thymine-(T), cytosine-(C), uracil-(U), or guanine-(G) including nucleotide. A nucleotide analog may comprise one or more differences or modifications relative to a natural nucleotide. Nonstandard nucleotides, nucleotide analogs, and/or modified analogs may include, but are not limited to, diaminopurine, 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methyl cytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-D46-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid(v), 5-methyl-2-thiouracil, 3-(3-amino-3-N-2-carboxypropyl) uracil, (acp3)w, 2,6-diaminopurine, ethynyl nucleotide bases, 1-propynyl nucleotide bases, azido nucleotide bases, phosphoroselenoate nucleic acids, and modified versions thereof (e.g., by oxidation, reduction, and/or addition of a substituent such as an alkyl, hydroxyalkyl, hydroxyl, or halogen moiety). Nucleic acid molecules (e.g., polynucleotides, double-stranded nucleic acid molecules, single-stranded nucleic acid molecules, primers, adapters, etc.) may be modified at the base moiety (e.g., at one or more atoms that typically are available to form a hydrogen bond with a complementary nucleotide and/or at one or more atoms that are not typically capable of forming a hydrogen bond with a complementary nucleotide), sugar moiety, or phosphate backbone. In some cases, a nucleotide may include a modification in its phosphate moiety, including a modification to a triphosphate moiety. Additional, non-limiting examples of modifications include phosphate chains of greater length (e.g., a phosphate chain having, 4, 5, 6, 7, 8, 9, 10 or more phosphate moieties), modifications with thiol moieties (e.g., alpha-thio triphosphate and beta-thiotriphosphates), and modifications with selenium moieties (e.g., phosphoroselenoate nucleic acids). A nucleotide or nucleotide analog may comprise a sugar selected from the group consisting of ribose, deoxyribose, and modified versions thereof (e.g., by oxidation, reduction, and/or addition of a substituent such as an alkyl, hydroxyalkyl, hydroxyl, or halogen moiety). A nucleotide analog may also comprise a modified linker moiety (e.g., in lieu of a phosphate moiety). Nucleotide analogs may also contain amine-modified groups, such as aminoallyl-dUTP (aa-dUTP) and aminohexylacrylamide-dCTP (aha-dCTP) to allow covalent attachment of amine reactive moieties, such as N-hydroxysuccinimide esters (NHS). Alternatives to standard DNA base pairs or RNA base pairs in the oligonucleotides of the present disclosure may provide, for example, higher density in bits per cubic mm, higher safety (resistant to accidental or purposeful synthesis of natural toxins), easier discrimination in photo-programmed polymerases, and/or lower secondary structure. Nucleotide analogs may be capable of reacting or bonding with detectable moieties for nucleotide detection.

The term “homopolymer,” as used herein, generally refers to a polymer or a portion of a polymer comprising identical monomer units. A homopolymer may have a homopolymer sequence. A nucleic acid homopolymer may refer to a polynucleotide or an oligonucleotide comprising consecutive repetitions of a same nucleotide or any nucleotide variants thereof. For example, a homopolymer can be poly(dA), poly(dT), poly(dG), poly(dC), poly(rA), poly(U), poly(rG), or poly(rC). A homopolymer can be of any length. For example, the homopolymer can have a length of at least 2, 3, 4, 5, 10, 20, 30, 40, 50, 100, 200, 300, 400, 500, or more nucleic acid bases. The homopolymer can have from 10 to 500, or 15 to 200, or 20 to 150 nucleic acid bases. The homopolymer can have a length of at most 500, 400, 300, 200, 100, 50, 40, 30, 20, 10, 5, 4, 3, or 2 nucleic acid bases. A molecule, such as a nucleic acid molecule, can include one or more homopolymer portions and one or more non-homopolymer portions. The molecule may be entirely formed of a homopolymer, multiple homopolymers, or a combination of homopolymers and non-homopolymers. In nucleic acid sequencing, multiple nucleotides can be incorporated into a homopolymeric region of a nucleic acid strand. Such nucleotides may be non-terminated to permit incorporation of consecutive nucleotides (e.g., during a single nucleotide flow).

The term “denaturation,” as used herein, generally refers to separation of a double-stranded molecule (e.g., DNA) into single-stranded molecules. Denaturation may be complete or partial denaturation. In partial denaturation, a single-stranded region may form in a double-stranded molecule by denaturation of the two deoxyribonucleic acid (DNA) strands flanked by double-stranded regions in DNA.

The term “melting temperature” or “melting point,” as used herein, generally refers to the temperature at which at least a portion of a strand of a nucleic acid molecule in a sample has separated from at least a portion of a complementary strand. The melting temperature may be the temperature at which a double-stranded nucleic acid molecule has partially or completely denatured. The melting temperature may refer to a temperature of a sequence among a plurality of sequences of a given nucleic acid molecule, or a temperature of the plurality of sequences. Different regions of a double-stranded nucleic acid molecule may have different melting temperatures. For example, a double-stranded nucleic acid molecule may include a first region having a first melting point and a second region having a second melting point that is higher than the first melting point. Accordingly, different regions of a double-stranded nucleic acid molecule may melt (e.g., partially denature) at different temperatures. The melting point of a nucleic acid molecule or a region thereof (e.g., a nucleic acid sequence) may be determined experimentally (e.g., via a melt analysis or other procedure) or may be estimated based upon the sequence and length of the nucleic acid molecule. For example, a software program such as MELTING may be used to estimate a melting temperature for a nucleic acid sequence (Dumousseau M, Rodriguez N, Juty N, Le Novére N, MELTING, a flexible platform to predict the melting temperatures of nucleic acids. BMC Bioinformatics. 2012 May 16; 13:101. doi: 10.1186/1471-2105-13-101). Accordingly, a melting point as described herein may be an estimated melting point. A true melting point of a nucleic acid sequence may vary based upon the sequences or lack thereof adjacent to the nucleic acid sequence of interest as well as other factors.

The term “sequencing,” as used herein, generally refers to a process for generating or identifying a sequence of a biological molecule, such as a nucleic acid molecule or a polypeptide. Such sequence may be a nucleic acid sequence, which may include a sequence of nucleic acid bases (e.g., nucleobases). Sequencing may be, for example, single molecule sequencing, sequencing by synthesis, sequencing by hybridization, or sequencing by ligation. Sequencing may be performed using template nucleic acid molecules immobilized on a support, such as a flow cell or one or more beads. A sequencing assay may yield one or more sequencing reads corresponding to one or more template nucleic acid molecules.

The term “read,” as used herein, generally refers to a nucleic acid sequence, such as a sequencing read. A sequencing read may be an inferred sequence of nucleic acid bases (e.g., nucleotides) or base pairs obtained via a nucleic acid sequencing assay. A sequencing read may be generated by a nucleic acid sequencer, such as a massively parallel array sequencer (e.g., Illumina or Pacific Biosciences of California). A sequencing read may correspond to a portion, or in some cases all, of a genome of a subject. A sequencing read may be part of a collection of sequencing reads, which may be combined through, for example, alignment (e.g., to a reference genome), to yield a sequence of a genome of a subject.

The term “support,” as used herein, generally refers to any solid or semi-solid article to which reagents such as nucleic acid molecules may be coupled (e.g., immobilized). Nucleic acid molecules may be synthesized, attached, ligated, or otherwise immobilized. Nucleic acid molecules may be coupled to (e.g., immobilized on) a support by any method including, but not limited to, physical adsorption, by ionic or covalent bond formation, or combinations thereof. A support may be 2-dimensional (e.g., a planar 2D support) or 3-dimensional. In some cases, a support may be a component of a flow cell and/or may be included within or adapted to be received by a sequencing instrument. A support may include a polymer, a glass, or a metallic material. Examples of supports include a membrane, a planar support, a microtiter plate, a bead (e.g., a magnetic bead), a filter, a test strip, a slide, a cover slip, and a test tube. A support may comprise organic polymers such as polystyrene, polyethylene, polypropylene, polyfluoroethylene, polyethyleneoxy, and polyacrylamide (e.g., polyacrylamide gel), as well as co-polymers and grafts thereof. A support may comprise latex or dextran. A support may also be inorganic, such as glass, silica, gold, controlled-pore-glass (CPG), or reverse-phase silica. The configuration of a support may be, for example, in the form of beads, spheres, particles, granules, a gel, a porous matrix, or a support. In some cases, a support may be a single solid or semi-solid article (e.g., a single particle), while in other cases a support may comprise a plurality of solid or semi-solid articles (e.g., a collection of particles). Supports may be planar, substantially planar, or non-planar. Supports may be porous or non-porous, and may have swelling or non-swelling characteristics. A support may be shaped to comprise one or more wells, depressions, or other containers, vessels, features, or locations. A plurality of supports may be configured in an array at various locations. A support may be addressable (e.g., for robotic delivery of reagents), or by detection approaches, such as scanning by laser illumination and confocal or deflective light gathering. For example, a support may be in optical and/or physical communication with a detector. Alternatively, a support may be physically separated from a detector by a distance. An amplification support (e.g., a bead) can be placed within or on another support (e.g., within a well of a second support).

The term “coupled to,” as used herein, generally refers to an association between two or more objects that may be temporary or substantially permanent. A first object may be reversibly or irreversibly coupled to a second object. For example, a nucleic acid molecule may be reversibly coupled to a particle. A reversible coupling may comprise, for example, a releasable coupling (e.g., in which a first object may be released from a second object to which the first object is coupled). A first object releasably coupled to a second object may be separated from the second object, e.g., upon application of a stimulus, which stimulus may comprise a photo stimulus (e.g., ultraviolet light), a thermal stimulus, a chemical stimulus (e.g., reducing agent), or any other useful stimulus. Coupling may encompass immobilization to a support (e.g., as described herein). Similarly, coupling may encompass attachment, such as attachment of a first object to a second object. A coupling may comprise any interaction that affects an association between two objects, including, for example, a covalent bond, a non-covalent interaction (e.g., electrostatic interaction [e.g., hydrogen bonding, ionic interaction, and halogen bonding], π-interaction [e.g., π-π interaction, polar-π interaction, cation-π interaction, and anion-π interaction], van der Waals force-based interactions [e.g., dipole-dipole interactions, dipole-induced dipole interactions, and induced dipole-induced dipole interactions], hydrophobic interaction), a magnetic interaction (e.g., magnetic dipole-dipole interaction, indirect dipole-dipole coupling), an electromagnetic interaction, adsorption, or any other useful interaction. For example, a particle may be coupled to a planar support via an electrostatic interaction. In another example, a particle may be coupled to a planar support via a magnetic interaction. In another example, a particle may be coupled to a planar support via a covalent interaction. Similarly, a nucleic acid molecule may be coupled to a particle via a covalent interaction. Alternatively or additionally, a nucleic acid molecule may be coupled to a particle via a non-covalent interaction. A coupling between a first object and a second object may comprise a labile moiety, such as a moiety comprising an ester, vicinal diol, phosphodiester, peptidic, glycosidic, sulfone, Diels-Alder, or similar linkage. The strength of a coupling between a first object and a second object may be indicated by a dissociation constant, Kd, that indicates the inclination of a coupled object comprising a first object and a second object to dissociate into the uncoupled first and second objects and may be expressed as a ratio of dissociated (e.g., uncoupled) objects to coupled objects. A smaller dissociation constant is generally indicative of a stronger coupling between coupled objects.

Coupled objects and their corresponding uncoupled components may exist in dynamic equilibrium with one another. For example, a solution comprising a plurality of coupled objects each comprising a first object and a second object may also include a plurality of first objects and a plurality of second objects. At a given point in time, a given first object and a given second object may be coupled to one another or the objects may be uncoupled; the relative concentrations of coupled and uncoupled components throughout the solution will depend upon the strength of the coupling between the first and second objects (reflected in the dissociation constant). For example, a binding moiety may be coupled to a nucleic acid molecule to provide a binding complex. In a solution comprising a plurality of binding complexes each comprising a binding moiety coupled to a nucleic acid molecule, the plurality of binding complexes may exist in equilibrium with their constituent nucleic acid molecules and binding moieties. The association between a given nucleic acid molecule and a given binding moiety may be such that, at a given point in time, at least 50%, such as at least 60%, 65%, 70%, 75%, 80%, 85%, 90%, 95%, 98%, or more, of the nucleic acid molecules may be components of a binding complex of the plurality of binding complexes.

The term “label,” as used herein, generally refers to a moiety that is capable of coupling with a species, such as, for example a nucleotide analog. A label may include an affinity moiety. In some cases, a label may be a detectable label that emits a signal (or reduces an already emitted signal) that can be detected. In some cases, such a signal may be indicative of incorporation of one or more nucleotides or nucleotide analogs. In some cases, a label may be coupled to a nucleotide or nucleotide analog, which nucleotide or nucleotide analog may be used in a primer extension reaction. In some cases, the label may be coupled to a nucleotide analog after a primer extension reaction. The label, in some cases, may be reactive specifically with a nucleotide or nucleotide analog. Coupling may be covalent or non-covalent (e.g., via ionic interactions, Van der Waals forces, etc.). In some cases, coupling may be via a linker, which may be cleavable, such as photo-cleavable (e.g., cleavable under ultra-violet light), chemically-cleavable (e.g., via a reducing agent, such as dithiothreitol (DTT), tris(2-carboxyethyl)phosphine (TCEP), tris(hydroxypropyl)phosphine (THP) or enzymatically cleavable (e.g., via an esterase, lipase, peptidase or protease). In some cases, the label may be luminescent; that is, fluorescent or phosphorescent. For example, the label may be or comprise a fluorescent moiety (e.g., a dye). Dyes and labels may be incorporated into nucleic acid sequences. Dyes and labels may also be incorporated into or attached to linkers, such as linkers for linking one or more beads to one another. For example, labels such as fluorescent moieties may be linked to nucleotides or nucleotide analogs via a linker (e.g., as described herein). Non-limiting examples of dyes include SYBR green, SYBR blue, DAPI, propidium iodine, Hoechst, SYBR gold, ethidium bromide, acridine, proflavine, acridine orange, acriflavine, fluorocoumarin, ellipticine, daunomycin, chloroquine, distamycin D, chromomycin, homidium, mithramycin, ruthenium polypyridyls, anthramycin, phenanthridines and acridines, propidium iodide, hexidium iodide, dihydroethidium, ethidium homodimer-1 and -2, ethidium monoazide, ACMA, Hoechst 33258, Hoechst 33342, Hoechst 34580, DAPI, acridine orange, 7-AAD, actinomycin D, LDS751, hydroxystilbamidine, SYTOX Blue, SYTOX Green, SYTOX Orange, POPO-1, POPO-3, YOYO-1, YOYO-3, TOTO-1, TOTO-3, JOJO-1, LOLO-1, BOBO-1, BOBO-3, PO-PRO-1, PO-PRO-3, BO-PRO-1, BO-PRO-3, TO-PRO-1, TO-PRO-3, TO-PRO-5, JO-PRO-1, LO-PRO-1, YO-PRO-1, YO-PRO-3, PicoGreen, OliGreen, RiboGreen, SYBR Gold, SYBR Green I, SYBR Green II, SYBR DX, SYTO labels (e.g., SYTO-40, -41, -42, -43, -44, and -45 (blue); SYTO-13, -16, -24, -21, -23, -12, -11, -20, -22, -15, -14, and -25 (green); SYTO-81, -80, -82, -83, -84, and-85 (orange); and SYTO-64, -17, -59, -61, -62, -60, and -63 (red)), fluorescein, fluorescein isothiocyanate (FITC), tetramethyl rhodamine isothiocyanate (TRITC), rhodamine, tetramethyl rhodamine, R-phycoerythrin, Cy-2, Cy-3, Cy-3.5, Cy-5, Cy5.5, Cy-7, Texas Red, Phar-Red, allophycocyanin (APC), Sybr Green I, Sybr Green II, Sybr Gold, CellTracker Green, 7-AAD, ethidium homodimer I, ethidium homodimer II, ethidium homodimer III, ethidium bromide, umbelliferone, eosin, green fluorescent protein, erythrosin, coumarin, methyl coumarin, pyrene, malachite green, stilbene, lucifer yellow, cascade blue, dichlorotriazinylamine fluorescein, dansyl chloride, fluorescent lanthanide complexes such as those including europium and terbium, carboxy tetrachloro fluorescein, 5 and/or 6-carboxy fluorescein (FAM), VIC, 5- (or 6-) iodoacetamidofluorescein, 5-{[2 (and 3)-5-(Acetylmercapto)-succinyl]amino} fluorescein (SAMSA-fluorescein), lissamine rhodamine B sulfonyl chloride, 5 and/or 6 carboxy rhodamine (ROX), 7-amino-methyl-coumarin, 7-Amino-4-methylcoumarin-3-acetic acid (AMCA), BODIPY fluorophores, 8-methoxypyrene-1,3,6-trisulfonic acid trisodium salt, 3,6-Disulfonate-4-amino-naphthalimide, phycobiliproteins, AlexaFluor labels (e.g., AlexaFluor 350, 405, 430, 488, 532, 546, 555, 568, 594, 610, 633, 635, 647, 660, 680, 700, 750, and 790 dyes), DyLight labels (e.g., DyLight 350, 405, 488, 550, 594, 633, 650, 680, 755, and 800 dyes), Black Hole Quencher Dyes (Biosearch Technologies) (e.g., BH1-0, BHQ-1, BHQ-3, and BHQ-10), QSY Dye fluorescent quenchers (Molecular Probes/Invitrogen) (e.g., QSY7, QSY9, QSY21, and QSY35), Dabcyl, Dabsyl, Cy5Q, Cy7Q, Dark Cyanine dyes (GE Healthcare), Dy-Quenchers (Dyomics) (e.g., DYQ-660 and DYQ-661), ATTO fluorescent quenchers (ATTO-TEC GmbH) (e.g., ATTO 540Q, ATTO 580Q, ATTO 612Q, Atto532 [e.g., Atto 532 succinimidyl ester], and Atto633), and other fluorophores and/or quenchers. Additional examples are included in structures provided herein. Dyes included in structures provided herein are contemplated for use in combination with any linker and substrate described herein. A fluorescent dye may be excited by application of energy corresponding to the visible region of the electromagnetic spectrum (e.g., between about 430-770 nanometers (nm)). Excitation may be done using any useful apparatus, such as a laser and/or light emitting diode. Optical elements including, but not limited to, mirrors, waveplates, filters, monochromators, gratings, beam splitters, and lenses may be used to direct light to or from a fluorescent dye. A fluorescent dye may emit light (e.g., fluoresce) in the visible region of the electromagnetic spectrum (e.g., between about 430-770 nm). A fluorescent dye may be excited over a single wavelength or a range of wavelengths. A fluorescent dye may be excitable by light in the red region of the visible portion of the electromagnetic spectrum (about 625-740 nm) (e.g., have an excitation maximum in the red region of the visible portion of the electromagnetic spectrum). Alternatively or additionally, fluorescent dye may be excitable by light in the green region of the visible portion of the electromagnetic spectrum (about 500-565 nm) (e.g., have an excitation maximum in the green region of the visible portion of the electromagnetic spectrum). A fluorescent dye may emit signal in the red region of the visible portion of the electromagnetic spectrum (about 625-740 nm) (e.g., have an emission maximum in the red region of the visible portion of the electromagnetic spectrum). Alternatively or additionally, fluorescent dye may emit signal in the green region of the visible portion of the electromagnetic spectrum (about 500-565 nm) (e.g., have an emission maximum in the green region of the visible portion of the electromagnetic spectrum).

Labels may be quencher molecules. The term “quencher,” as used herein, generally refers to molecules that may be energy acceptors. A quencher may be a molecule that can reduce an emitted signal. For example, a template nucleic acid molecule may be designed to emit a detectable signal. Incorporation of a nucleotide or nucleotide analog comprising a quencher can reduce or eliminate the signal, which reduction or elimination is then detected. Luminescence from labels (e.g., fluorescent moieties, such as fluorescent moieties linked to nucleotides or nucleotide analogs) may also be quenched (e.g., by incorporation of other nucleotides that may or may not comprise labels). In some cases, as described elsewhere herein, labelling with a quencher can occur after nucleotide or nucleotide analog incorporation (e.g., after incorporation of a nucleotide or nucleotide analog comprising a fluorescent moiety). In some cases, the label may be a type that does not self-quench or exhibit proximity quenching. Non-limiting examples of a label type that does not self-quench or exhibit proximity quenching include Bimane derivatives such as Monobromobimane. The term “proximity quenching,” as used herein, generally refers to a phenomenon where one or more dyes near each other may exhibit lower fluorescence as compared to the fluorescence they exhibit individually. In some cases, the dye may be subject to proximity quenching wherein the donor dye and acceptor dye are within 1 nm to 50 nm of each other. Examples of quenchers include, but are not limited to, Black Hole Quencher Dyes (Biosearch Technologies) (e.g., BH1-0, BHQ-1, BHQ-3, and BHQ-10), QSY Dye fluorescent quenchers (Molecular Probes/Invitrogen) (e.g., QSY7, QSY9, QSY21, and QSY35), Dabcyl, Dabsyl, Cy5Q, Cy7Q, Dark Cyanine dyes (GE Healthcare), Dy-Quenchers (Dyomics) (e.g., DYQ-660 and DYQ-661), and ATTO fluorescent quenchers (ATTO-TEC GmbH) (e.g., ATTO 540Q, ATTO 580Q, and ATTO 612Q). Fluorophore donor molecules may be used in conjunction with a quencher. Examples of fluorophore donor molecules that can be used in conjunction with quenchers include, but are not limited to, fluorophores such as Cy3B, Cy3, or Cy5; Dy-Quenchers (Dyomics) (e.g., DYQ-660 and DYQ-661); and ATTO fluorescent quenchers (ATTO-TEC GmbH) (e.g., ATTO 540Q, ATTO 580Q, and ATTO 612Q).

The term “labeling fraction,” as used herein, generally refers to the ratio of dye-labeled nucleotide or nucleotide analog to natural/unlabeled nucleotide or nucleotide analog of a single canonical type in a flow solution. The labeling fraction can be expressed as the concentration of the labeled nucleotide or nucleotide analog divided by the sum of the concentrations of labeled and unlabeled nucleotide or nucleotide analog. The labeling fraction may be expressed as a % of labeled nucleotides included in a solution (e.g., a nucleotide flow). The labeling fraction may be at least about 0.5%, 1%, 2%, 3%, 4%, 5%, 10%, 15%, 20%, 30%, 40%, 50%, 60%, 70%, 80%, 90%, or higher. For example, the labeling fraction may be at least about 20%. The labeling fraction may be about 100%. The labeling fraction may also be expressed as a ratio of labeled nucleotides to unlabeled nucleotides included in a solution. For example, the ratio of labeled nucleotides to unlabeled nucleotides may be at least about 1:5, 1:4, 1:3, 1:2, 1:1, 2:1, 3:1, 4:1, 5:1, or higher. For example, the ratio of labeled nucleotides to unlabeled nucleotides may be at least about 1:4. For example, the ratio of labeled nucleotides to unlabeled nucleotides may be at least about 1:1. For example, the ratio of labeled nucleotides to unlabeled nucleotides may be at least about 5:1.

The term “labeled fraction,” as used herein, generally refers to the actual fraction of labeled nucleic acid (e.g., DNA) resulting after treatment of a primer-template with a mixture of the dye-labeled and natural nucleotide or nucleotide analog. The labeled fraction may be about the same as the labeling fraction. For example, if 20% of nucleotides in a nucleotide flow are labeled, about 20% of nucleotides incorporated into a growing nucleic acid strand (e.g., during nucleic acid sequencing) may be labeled. Alternatively, the labeled fraction may be greater than the labeled fraction. For example, if 20% of nucleotides in a nucleotide flow are labeled, greater than 20% of nucleotides incorporated into a growing nucleic acid strand (e.g., during nucleic acid sequencing) may be labeled. Alternatively, the labeled fraction may be less than the labeled fraction. For example, if 20% of nucleotides in a nucleotide flow are labeled, less than 20% of nucleotides incorporated into a growing nucleic acid strand (e.g., during nucleic acid sequencing) may be labeled.

When a solution including less than 100% labeled nucleotides or nucleotide analogs is used in an incorporation process such as a sequencing process (e.g., as described herein), both labeled (“bright”) and unlabeled (“dark”) nucleotides or nucleotide analogs may be incorporated into a growing nucleic acid strand. The term “tolerance,” as used herein, generally refers to the ratio of the labeled fraction (e.g., “bright” incorporated fraction) to the labeling fraction (e.g., “bright” fraction in solution). For example, if a labeling fraction of 0.2 is used resulting in a labeled fraction of 0.4 the tolerance is 2. Similarly, if an incorporation process such as a sequencing process is performed using 2.5% labeled fraction in solution (b_{f}, bright solution fraction) and 5% is labeled (bk, bright incorporated fraction), the tolerance may be 2 (e.g., tolerance). This model may be linear for low labeling fractions (e.g., 10% or lower labeling fraction). For higher labeling fractions, tolerance may take into account competing dark incorporation. Tolerance may refer to a comparison of the ratio of bright incorporated fraction to dark incorporated fraction (bᵢ/dᵢ) to the ratio of bright solution fraction to dark solution fraction (b_{f}/d_{f}):

$$Tolerance = \frac{{b}_{i} / {d}_{i}}{{b}_{f} / {d}_{f}}$$

where dᵢ=1−bᵢ (e.g., dark incorporated fraction and bright incorporated fraction sum to 1 assuming 100% bright fraction is normalized to 1)

Though dᵢ cannot easily be measured, the bright incorporated fraction, can be measured (e.g., as described herein) and used to determine tolerance by fitting a curve of bright solution fraction (b_{f}) vs. bright incorporated fraction (N):

$${b}_{i} = \frac{tol ( {b}_{f} / {d}_{f} )}{1 + tol ( {b}_{f} / {d}_{f} )}$$

A “positive” tolerance number (>1) indicates that at 50% labeling fraction, more than 50% is labeled. A “negative” tolerance number (<1) indicates that at 50% labeling fraction, less than 50% is labeled.

The term “context,” as used herein, generally refers to the sequence of the neighboring nucleotides, or context, has been observed to affect the tolerance in an incorporation reaction. The nature of the enzyme, the pH and other factors may also affect the tolerance. Reducing context effects to a minimum greatly simplifies base determination.

The term “misincorporation,” as used herein, generally refers to occurrences when the DNA polymerase incorporates a nucleotide, either labeled or unlabeled, that is not the correct Watson-Crick partner for the template base. Misincorporation can occur more frequently in methods that lack competition of all four bases in an incorporation event, and leads to strand loss, and thus limits the read length of a sequencing method.

The term “mispair extension”, as used herein, generally refers to occurrences when the DNA polymerase incorporates a nucleotide, either labeled or unlabeled, that is not the correct Watson-Crick partner for the template base, then subsequently incorporates the correct Watson-Crick partner for the following base. Mispair extension generally results in lead phasing and limits the read length of a sequencing method.

Regarding quenching, dye-dye quenching between two dye moieties linked to different nucleotides (e.g., adjacent nucleotides in a growing nucleic acid strand, or nucleotides in a nucleic acid strand that are separated by one or more other nucleotides) may be strongly dependent on the distance between the two dye moieties. The distance between two dye moieties may be at least partially dependent on the properties of linkers connecting the two dye moieties to respective nucleotides or nucleotide analogs, including the linker compositions and functional lengths. Features of the linkers, including composition and functional length, may be affected by temperature, solvent, pH and salt concentration (e.g., within a solution). Quenching may also vary based on the nature of the dyes used. Quenching may also take place between dye moieties and nucleobase moieties (e.g., between a fluorescent dye and a nucleobase of a nucleotide with which the fluorescent dye is associated). Controlling quenching phenomena may be a key feature of the methods described herein.

Regarding flows, a nucleotide flow can consist of a mixture of labeled and unlabeled nucleotides or nucleotide analogs (e.g., nucleotides or nucleotide analogs of a single canonical type). For example, a solution comprising a plurality of optically (e.g., fluorescently) labeled nucleotides and a plurality of unlabeled nucleotides may be contacted with, e.g., a sequencing template (as described herein). The plurality of optically labeled nucleotides and a plurality of unlabeled nucleotides may each comprise the same canonical nucleotide or nucleotide analog. A flow may include only labeled nucleotides or nucleotide analogs. Alternatively, a flow may include only unlabeled nucleotides or nucleotide analogs. A flow may include a mixture of nucleotide or nucleotide analogs of different types (e.g., A and G).

A wash flow (e.g., a solution comprising a buffer) may be used to remove any nucleotides that are not incorporated into a nucleic acid complex (e.g., a sequencing template, as described herein). A cleavage flow (e.g., a solution comprising a cleavage reagent) may be used to remove dye moieties (e.g., fluorescent dye moieties) from optically (e.g., fluorescently) labeled nucleotides or nucleotide analogs. In some cases, different dyes (e.g., fluorescent dyes) may be removable using different cleavage reagents. In other cases, different dyes (e.g., fluorescent dyes) may be removable using the same cleavage reagents. Cleavage of dye moieties from optically labeled nucleotides or nucleotide analogs may comprise cleavage of all or a portion of a linker connecting a nucleotide or nucleotide analog to a dye moiety.

The term “cycle,” as used herein, generally refers to a process in which a nucleotide flow, a wash flow, and a cleavage flow corresponding to each canonical nucleotide (e.g., dATP, dCTP, dGTP, and dTTP or dUTP, or modified versions thereof) are used (e.g., provided to a sequencing template, as described herein). Multiple cycles may be used to sequence and/or amplify a nucleic acid molecule. The order of nucleotide flows can be varied.

Phasing can be lead or lag phasing. Lead phasing generally refers to the phenomenon in which a population of strands show incorporation of a nucleotide a flow ahead of the expected cycle (e.g., due to contamination in the system). Lag phasing refers to the phenomenon in which a population of strands shows incorporation of a nucleotide a flow behind the expected cycle (e.g., due to incompletion of extension in an earlier cycle).

The term “processing an analyte,” as used herein, generally refers to one or more stages of interaction with one more sample substances. Processing an analyte may comprise conducting a chemical reaction, biochemical reaction, enzymatic reaction, hybridization reaction, polymerization reaction, physical reaction, any other reaction, or a combination thereof with, in the presence of, or on, the analyte. Processing an analyte may comprise physical and/or chemical manipulation of the analyte. For example, processing an analyte may comprise detection of a chemical change or physical change, addition of or subtraction of material, atoms, or molecules, molecular confirmation, detection of the presence of a fluorescent label, detection of a Forster resonance energy transfer (FRET) interaction, or inference of absence of fluorescence. The term “analyte” may refer to molecules, cells, biological particles, or organisms. In some instances, a molecule may be a nucleic acid molecule, antibody, antigen, peptide, protein, or other biological molecule obtained from or derived from a biological sample. For example, an analyte may be a nucleic acid molecule. An analyte may originate from, and/or be derived from, a biological sample, such as from a cell or organism (e.g., as described herein). An analyte may be synthetic.

The term “detector,” as used herein, generally refers to a device that is capable of detecting or measuring a signal, such as a signal indicative of the presence or absence of an incorporated nucleotide or nucleotide analog, such as a nucleotide coupled to a fluorescent label (e.g., as described herein). A detector may detect multiple signals. One or more signals may be detected in real-time during, substantially during, or subsequent to a biological reaction, such as a sequencing reaction (e.g., sequencing comprising a primer extension reaction). A detector may include optical and/or electronic components that may detect and/or measure signals. Non-limiting examples of detection methods involving a detector include optical detection, spectroscopic detection, electrostatic detection, acoustic detection, magnetic detection, and electrochemical detection. Optical detection methods include, but are not limited to, light (e.g., UV-vis or infrared) absorption, light scattering, Rayleigh scattering, Raman scattering, surface enhanced Raman scattering, Mie scattering, fluorescence, luminescence, and phosphorescence. Spectroscopic detection methods include, but are not limited to, mass spectrometry, nuclear magnetic resonance (NMR) spectroscopy, and infrared spectroscopy. Electrostatic detection methods include, but are not limited to, gel-based techniques, such as, for example, gel electrophoresis. Electrochemical detection methods include, but are not limited to, electrochemical detection of amplified product after high-performance liquid chromatography separation of the amplified products. Detection may comprise continuous area scanning (e.g., as described herein).

Compounds and chemical moieties described herein, including linkers, may contain one or more asymmetric centers and thus give rise to enantiomers, diastereomers, and other stereoisomeric forms that are defined, in terms of absolute stereochemistry, as (R)- or (S)-, and, in terms of relative stereochemistry, as (D)- or (L)-. The D/L system relates molecules to the chiral molecule glyceraldehyde and is commonly used to describe biological molecules including amino acids. Unless stated otherwise, it is intended that all stereoisomeric forms of the compounds disclosed herein are contemplated by this disclosure. When the compounds described herein contain alkene double bonds, and unless specified otherwise, it is intended that this disclosure includes both E and Z geometric isomers (e.g., cis or trans.) Likewise, all possible isomers, as well as their racemic and optically pure forms, and all tautomeric forms are also intended to be included. The term “geometric isomer” refers to E or Z geometric isomers (e.g., cis or trans) of an alkene double bond. The term “positional isomer” refers to structural isomers around a central ring, such as ortho-, meta-, and para-isomers around a phenyl ring. Separation of stereoisomers may be performed by chromatography or by forming diastereomers and separating by recrystallization, or chromatography, or any combination thereof (Jean Jacques, Andre Collet, Samuel H. Wilen, “Enantiomers, Racemates and Resolutions”, John Wiley and Sons, Inc., 1981, herein incorporated by reference for this disclosure). Stereoisomers may also be obtained by stereoselective synthesis.

Compounds and chemical moieties described herein, including linkers, may exist as tautomers. A “tautomer” refers to a molecule wherein a proton shift from one atom of a molecule to another atom of the same molecule is possible. In circumstances where tautomerization is possible, a chemical equilibrium of the tautomers will exist. Unless otherwise stated, chemical structures depicted herein are intended to include structures which are different tautomers of the structures depicted. For example, the chemical structure depicted with an enol moiety also includes the keto tautomer form of the enol moiety. The exact ratio of the tautomers depends on several factors, including physical state, temperature, solvent, and pH. Some examples of tautomeric equilibrium include:

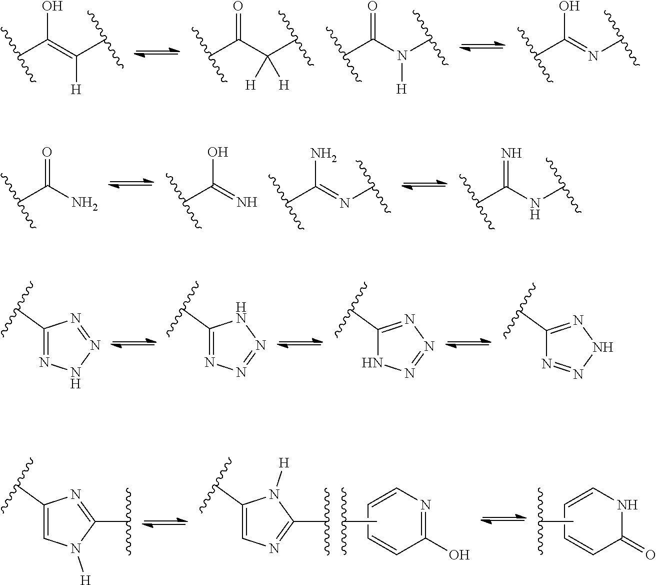

Compounds and chemical moieties described herein, including linkers and dyes, may be provided in different enriched isotopic forms. For example, compounds may be enriched in the content of ²H, ³H, ¹¹C, ¹³C and/or ¹⁴C. For example, a linker, substrate (e.g., nucleotide or nucleotide analog), or dye may be deuterated in at least one position. In some examples, a linker, substrate (e.g., nucleotide or nucleotide analog), or dye may be fully deuterated. Such deuterated forms can be made by the procedure described in U.S. Pat. Nos. 5,846,514 and 6,334,997, each of which are herein incorporated by reference in their entireties. As described in U.S. Pat. Nos. 5,846,514 and 6,334,997, deuteration can improve the metabolic stability and or efficacy, thus increasing the duration of action of drugs.

Unless otherwise stated, structures depicted and described herein are intended to include compounds which differ only in the presence of one or more isotopically enriched atoms. For example, compounds and chemical moieties having the present structures except for the replacement of a hydrogen by a deuterium or tritium, or the replacement of a carbon by ¹³C- or ¹⁴C-enriched carbon are within the scope of the present disclosure.

The compounds and chemical moieties of the present disclosure may contain unnatural proportions of atomic isotopes at one or more atoms that constitute such compounds. For example, a compound or chemical moiety such as a linker, substrate (e.g., nucleotide or nucleotide analog), or dye, or a combination thereof, may be labeled with one or more isotopes, such as deuterium (²H), tritium (³H), iodine-125 (¹²⁵I) or carbon-14 (¹⁴C). Isotopic substitution with ²H, ¹¹C, ¹³C, ¹⁴C, ¹⁵C, ¹²N, ¹³N, ¹⁵N, ¹⁶N, ¹⁶O, ¹⁷O, ¹⁴F, ¹⁵F, ¹⁶F, ¹⁷F, ¹⁸F, ³³S, ³⁴S, ³⁵S, ³⁶S, ³⁵Cl, ³⁷Cl, ⁷⁹Br, ⁸¹Br, and ¹²⁵I are all contemplated. All isotopic variations of the compounds and chemical moieties described herein, whether radioactive or not, are encompassed within the scope of the present disclosure.

Provided herein are methods, systems, and apparatus for high throughput sequencing, such as at an industrial scale. A sequencing system of the present disclosure may comprise a sequencing apparatus. A sequencing system of the present disclosure may comprise a plurality of sequencing apparatus. A sequencing system and/or sequencing apparatus of the present disclosure may comprise one or more stations for flexible control of individual stations and/or operations performed therein. For example, the one or more stations can include a sample station, a substrate station, a reagent station, a sequencing station, a processing station, and the like. In some instances, a station may be controlled independent of operations performed in other stations. In some instances, instructions to a station may be provided independent of operations performed in other stations. Beneficially, operation instructions may be provided, updated, adjusted, and/or cancelled in real-time, such as during sequencing.

### High-Throughput Sequencing Systems

The present disclosure provides sequencing systems, which sequencing systems may be used to process one or more nucleic acid samples. In some cases, a sequencing system provided herein may be used to process a plurality of nucleic acid samples in sequence or simultaneously. A sequencing system configured to process a plurality of nucleic acid samples may be considered a “high throughput” sequencing system.

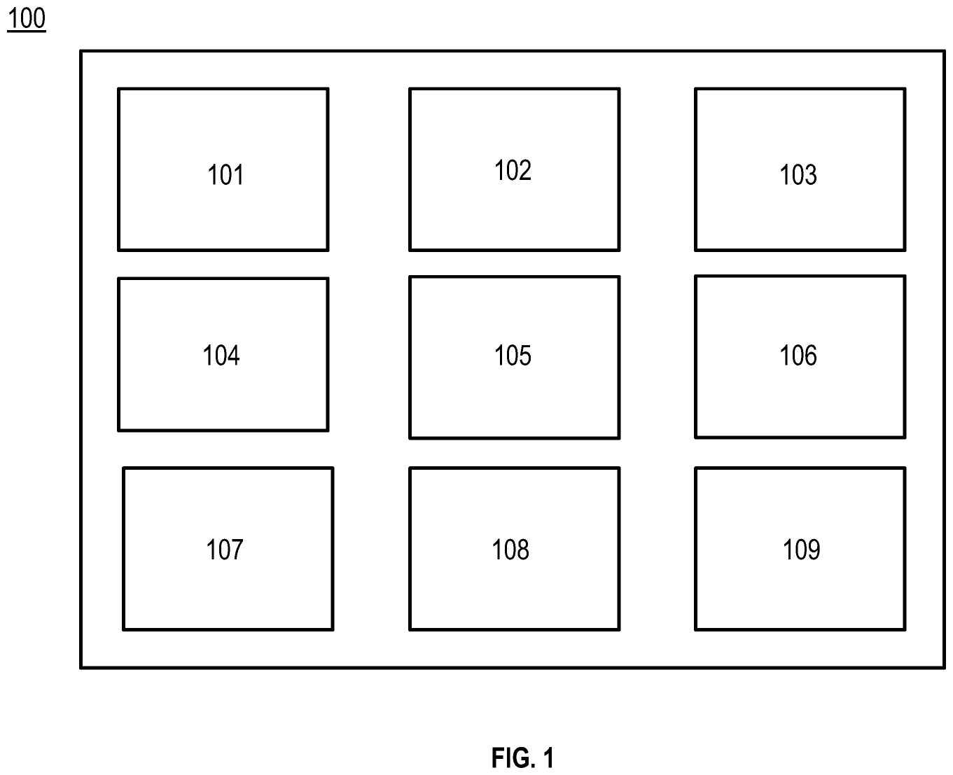

FIG. 1 illustrates a sequencing system 100, which sequencing system may be a high throughput sequencing system. The sequencing system 100 may comprise one or more stations 101, 102, 103, 104, 105, 106, 107, 108, and 109. While nine examples of stations are illustrated, it will be appreciated that there may be any number of stations in the system. In some instances, a station of the sequencing system, and/or operation performed therein, may be controlled independent of other operations and/or independent of other stations in the sequencing system. In some instances, two or more stations of the sequencing system may be controlled together and/or substantially simultaneously, such as with a single set of instructions.

For example, the sequencing system 100 may comprise one or more of a sample station 101, a substrate station 102, a reagent station 103, a processing station 104, a detection station 105, a diluent station 106, a controlling station 107, a power station 108, and an instructions station 109. In some cases, the system may comprise fewer stations. For example, one or more stations described above may not be included. In some cases, the system may comprise one or more additional stations.

The sample station 101 may be configured to receive and/or supply a sample to the processing station 104. A sample may comprise an analyte. For example, the sample may be a nucleic acid sample comprising a nucleic acid molecule (e.g., a deoxyribonucleic acid (DNA) or ribonucleic acid (RNA) molecule or a plurality of DNA and/or RNA molecules). In some examples, a sample may comprise a plurality of supports such as beads which may have one or more nucleic acid molecules (e.g., DNA and/or RNA molecules) immobilized thereon (e.g., on their surface). The sample may be according to the descriptions provided elsewhere herein. In some examples, the sample may undergo pre-processing prior to being supplied to or loaded on the sequencing system 100. For example, a sample may be subjected to a polymerase chain reaction (PCR) (e.g., emulsion PCR or “ePCR”) prior to being received by the sample stations or a tube thereof.

The sample station may comprise or be configured to receive a plurality of samples, such as a plurality of nucleic acid samples (e.g., as described herein). For example, a sample may be provided in a tube, well, or compartment in the sample station, or any other container that is capable of isolating a sample from other samples. In some cases, a sample may be provided on a support (e.g., as described herein).

In some instances, the sample station may comprise or be configured to receive at least about 1, 2, 3, 4, 5, 6, 7, 8, 9, 10, 20, 30, 40, 50, 60, 70, 80, 90, 100, 200, 300, 400, 500 or more samples. Alternatively or additionally, the sample station may comprise or be configured to receive at most about 500, 400, 300, 200, 100, 90, 80, 70, 60, 50, 40, 30, 20, 10, 9, 8, 7, 6, 5, 4, 3, 2, or 1 sample. In some instances, a sample may be derived or associated with a sample origin. In some instances, multiple samples may be derived or associated with the same sample origin. In some instances, a sample may be derived or associated with multiple sample origins. Multiple samples may be configured to be analyzed simultaneously (e.g., as described herein). For example, multiple samples may be configured to be included on a same support, such as a same array (e.g., substantially planar array). Such samples may be spatially separated on a support (e.g., at predefined spatial locations such as individually addressable locations, which locations may comprise wells and/or spatial patterning) and/or may be indexed using labels, barcodes, or other indices. Alternatively or additionally, samples may be configured to be analyzed separately.

Provided herein are systems and methods for loading a sample from the sample station 101. A sample may undergo one or more preparation (e.g., pre-processing) operations, such as one or more amplification reactions (e.g., one or more PCR processes, such as one or more ePCR processes), prior to input to the sample station. For example, the sample input to the sample station may be provided in a tube, as described elsewhere herein. The sample may comprise a plurality of particles (e.g., beads) in a solution, wherein a particle (e.g., bead) comprises a plurality of nucleic acid molecules coupled thereto (e.g., immobilized thereon). In some cases, each bead in the sample may comprise a distinct colony of amplification products (e.g., from PCR). The sample (e.g., via, or with aid of the tube) may be transferred to a substrate (e.g., a wafer), and may be dispensed over the substrate. In some cases, after dispensing, the sample may be given some time to settle on the substrate prior to performing further operations. Such time (e.g., incubation time or settlement time) may be at least about 1 minute (min), 5 min, 10 min, 15 min, 20 min, 25 min, 30 min, 40 min, 50 min, 60 min (1 hour (hr)), 70 min, 80 min, 90 min, 100 min, 120 min (2 hrs), or longer. The settlement time may allow the sample to couple with (e.g., immobilize thereon) the surface of the substrate (e.g., wafer).

A sample loading process on the sequencing system 100 may comprise providing and/or using a variety of systems, methods, and/or techniques. A loading system may comprise an interface such as a transport line, such as a pipe, tube, capillary, duct, channel, conduit, canal, line, or any other piece, device, equipment, or object which may be configured to receive, move, transport, and/or deliver the sample (e.g., to a substrate). The methods and systems may comprise a robotic interface and software to perform sample receipt and delivery. In some cases, the system may be compatible for use by a human operator. In some cases, a human operator may not be needed for performing the methods. In some cases, the methods and systems may be partially or fully automated. A sequencing system may comprise a system or mechanism for cleaning, decontaminating, and/or sanitizing all or a portion of the loading system that may be used to transfer sample material to a location for subsequent processing. For example, the system may include a mechanism for cleaning, decontaminating, and/or sanitizing a channel, capillary, duct, conduit, canal, line, or other material used to transfer sample material to a location for subsequent processing.

In some examples, sample loading on the system may be performed in one step. Alternatively, sample loading may be performed in more than one step. For example, sample loading may be performed in at least 1, 2, 3, 4, 5, 6, 7, 8, 9, 10, or more steps. For example, a first sample may be transferred to a location for analysis (e.g., a location including a substrate such as a wafer) in a first step and a second sample may be transferred to a location for analysis (e.g., the same or a different location) in a second step. In another example, a first sample may be transferred to a location for analysis (e.g., a location including a substrate such as a wafer) and a second sample may be transferred to a location for analysis (e.g., the same or a different location) in a same step (e.g., simultaneously and/or in a coordinated fashion).

In some cases, sample loading may comprise one or more feedback systems, such as involving monitoring (e.g., imaging) and control feedback. In an example, a sample or a portion thereof may be loaded on the substrate. For example, a sample comprising particles (e.g., beads) with nucleic acid molecules may be dispensed onto a substrate (e.g., wafer) comprising a patterned surface. A data set indicative of the status of loading may be collected from such load or loading operation. The data set may comprise any format, such as a signal, an image, or any other data type which may be capable of providing information about the status of the load, for example, information with respect to whether and how efficiently the beads have been properly loaded in predefined locations or areas, or other information indicative of the efficiency or quality of the load (e.g., first load). The data or information may be programmatically or manually analyzed to make decisions about the subsequent loads or subsequent loading steps. Adjustments to the subsequent loading procedures may be made as appropriate, and a subsequent loading process may be performed. For example, an operator may observe and evaluate the data (e.g., via a user interface) and make the decision about the subsequent load. Alternatively, the system may be automated in whole or in part. For example, the system may comprise an automated monitoring and control scheme which may provide feedback to the system for the subsequent loads or steps. The open substrate described in further detail elsewhere herein may facilitate flexibility and convenience for loading according to the methods provided herein.

The substrate station 102 may be configured to supply a substrate to the processing station. The substrate station may comprise a plurality of substrates (e.g., wafers, as described herein). For example, a substrate may be provided in a rack (e.g., horizontal or vertical) in the sample station, or in any other structure that is capable of isolating a substrate from other substrates. A substrate may be provided on or configured to be provided on (e.g., in direct physical contact with) a stage, which stage may be translated, rotated, or otherwise moved automatically or upon user input (e.g., as described herein). A substrate may be configured to be levitated (e.g., magnetically levitated). A substrate may be configured to be contacted at a fixed number of points, such as at a center of the substrate (e.g., a center of a disc-shaped substrate) to facilitate rotation of the substrate. A substrate may comprise an opening (e.g., a hole), depression, or other physical feature to facilitate transfer and/or movement of the substrate within the system. For example, the substrate may comprise an opening or depression at a center of the substrate (e.g., a center of a disc-shaped substrate) configured to facilitate interaction between the substrate and a component of the system configured to stabilize and, in some cases, rotate or otherwise move the substrate, such as a rotatable element. A system may comprise a mechanism for moving a substrate from a storage location such as a rack to the processing station, which mechanism may comprise, for example, a robotic arm.

In some instances, the substrate station may comprise at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, or more substrates. Alternatively or additionally, the substrate station may comprise at most about 50, 40, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 substrate. In some instances, the substrate station may comprise a uniform type of substrates. In some instances, the substrate station may comprise different types of substrate, such as differently patterned substrates, substrates comprising different materials, substrates of different sizes, etc.

Substrates of the present disclosure may be an open substrate. The term “open substrate”, as used herein, generally refers to a substantially planar substrate in which a single active surface is physically accessible at any point from a direction normal to the substrate. Substantially planar may refer to planarity at a micrometer level or nanometer level. Alternatively, substantially planar may refer to planarity at less than a nanometer level or greater than a micrometer level (e.g., millimeter level).

The substrate may be a solid substrate. Alternatively or additionally, the substrate may not be solid. The substrate may entirely or partially comprise one or more of glass, silicon, a metal such as aluminum, copper, titanium, chromium, or steel, a ceramic such as titanium oxide or silicon nitride, a plastic such as polyethylene (PE), low-density polyethylene (LDPE), high-density polyethylene (HDPE), polypropylene (PP), polystyrene (PS), high impact polystyrene (HIPS), polyvinyl chloride (PVC), polyvinylidene chloride (PVDC), acrylonitrile butadiene styrene (ABS), polyacetylene, polyamides, polycarbonates, polyesters, polyurethanes, polyepoxide, polymethyl methacrylate (PMMA), polytetrafluoroethylene (PTFE), phenol formaldehyde (PF), melamine formaldehyde (MF), urea-formaldehyde (UF), polyetheretherketone (PEEK), polyetherimide (PEI), polyimides, polylactic acid (PLA), furans, silicones, polysulfones, any mixture of any of the preceding materials, or any other appropriate material. The substrate may be entirely or partially coated with one or more layers of a metal such as aluminum, copper, silver, or gold, an oxide such as a silicon oxide (SiₓO_{y}, where x, y may take on any possible values), a photoresist such as SU8, a surface coating such as an aminosilane or hydrogel, polyacrylic acid, polyacrylamide dextran, polyethylene glycol (PEG), or any combination of any of the preceding materials, or any other appropriate coating. The one or more layers may have a thickness of at least 1 nanometer (nm), such as at least 2 nm, at least 5 nm, at least 10 nm, at least 20 nm, at least 50 nm, at least 100 nm, at least 200 nm, at least 500 nm, at least 1 micrometer (μm), at least 2 μm, at least 5 μm, at least 10 μm, at least 20 μm, at least 50 μm, at least 100 μm, at least 200 μm, at least 500 μm, at least 1 millimeter (mm), or more. The one or more layers may have a thickness that is within a range defined by any two of the preceding values.

The substrate may have any shape, form, or dimension. In some instances, for example, the substrate may have the general form of a cylinder, a cylindrical shell or disk, a wafer, a rectangular prism, or any other geometric form. The substrate may have a thickness (e.g., a minimum dimension) of at least about 100 μm, 200 μm, 300 μm, 400 μm, 500 μm, 1 mm, 2 mm, 3 mm, 4 mm, 5 mm, 1 centimeter (cm), 2 cm, 3 cm, 4 cm, 5 cm or more. The substrate may have a thickness that is within a range defined by any two of the preceding values. The substrate may have a first lateral dimension (such as a width for a substrate having the general form of a rectangular prism or a radius for a substrate having the general form of a cylinder) of at least about 1 mm, 2 mm, 3 mm, 4 mm, 5 mm, 1 cm, 2 cm, 3 cm, 4 cm, 5 cm, 6 cm, 7 cm, 8 cm, 9 cm, 10 cm, 20 cm, 30 cm, 40 cm, 50 cm, 60 cm, 70 cm, 80 cm, 90 cm, 1 meter (m), or more. The substrate may have a first lateral dimension that is within a range defined by any two of the preceding values. The substrate may have a second lateral dimension (such as a length for a substrate having the general form of a rectangular prism) or at least at least about 1 mm, 2 mm, 3 mm, 4 mm, 5 mm, 1 cm, 2 cm, 3 cm, 4 cm, 5 cm, 6 cm, 7 cm, 8 cm, 9 cm, 10 cm, 20 cm, 30 cm, 40 cm, 50 cm, 60 cm, 70 cm, 80 cm, 90 cm, 1 meter (m) or more. The substrate may have a second lateral dimension that is within a range defined by any two of the preceding values. A surface of the substrate may be planar or substantially planar. Alternatively or additionally, a surface of the substrate may be textured or patterned. For example, the substrate may comprise grooves, troughs, hills, and/or pillars. In some instances, the substrate may comprise wells. In some instances, the substrate may define one or more cavities (e.g., micro-scale cavities or nano-scale cavities). The substrate may have a regular textures and/or patterns across the surface of the substrate. For example, the substrate may have regular geometric structures (e.g., wedges, cuboids, cylinders, spheroids, hemispheres, etc.) above or below a reference level of the surface. Alternatively, the substrate may have irregular textures and/or patterns across the surface of the substrate. For example, the substrate may have any arbitrary structure above or below a reference level of the substrate. In some instances, a texture of the substrate may comprise structures having a maximum dimension of at most about 100%, 90%, 80%, 70%, 60%, 50%, 40%, 30%, 20%, 10%, 9%, 8%, 7%, 6%, 5%, 4%, 3%, 2%, 1%, 0.1%, 0.01%, 0.001%, 0.0001%, 0.00001% of the total thickness of the substrate or a layer of the substrate. In some instances, the textures and/or patterns of the substrate may define at least part of an individually addressable location on the substrate. A textured and/or patterned substrate may be substantially planar.

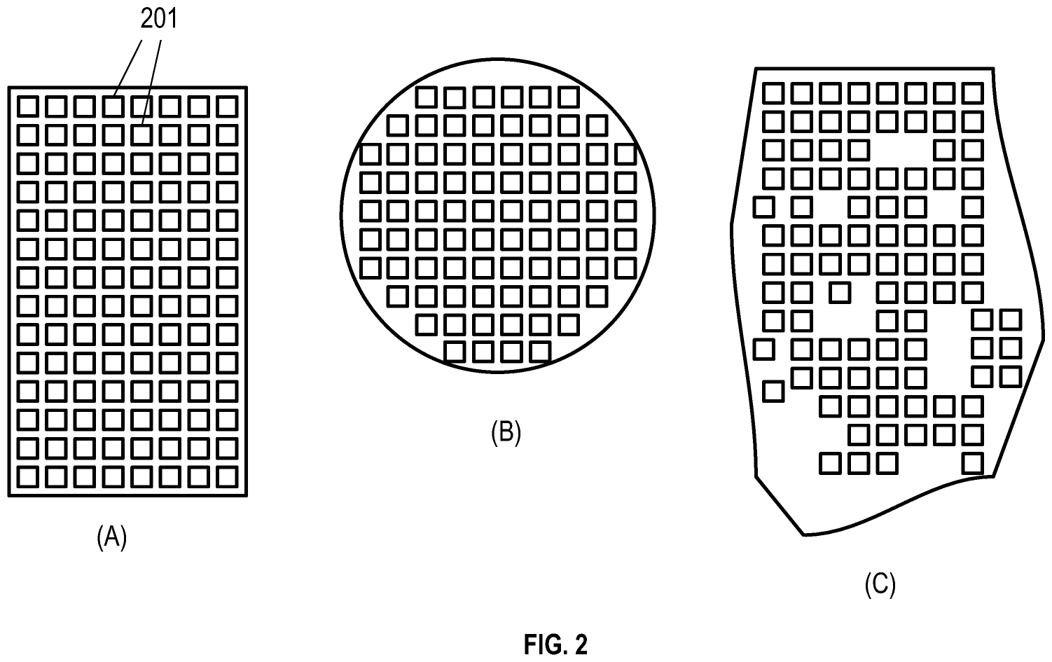

The substrate may comprise an array. For instance, the array may be located on a lateral surface of the substrate. The array may be a planar array. The array may have the general shape of a circle, annulus, rectangle, or any other shape. The array may comprise linear and/or non-linear rows. The array may be evenly spaced or distributed. The array may be arbitrarily spaced or distributed. The array may have regular spacing. The array may have irregular spacing. The array may be a textured array. The array may be a patterned array. FIG. 2 illustrates examples of arrays of individually addressable locations 201 on a substrate (e.g., from a top view), with panel A showing a substantially rectangular substrate with regular linear arrays, panel B showing a substantially circular substrate with regular linear arrays, and panel C showing an arbitrarily shaped substrate with irregular arrays. In an example, an array may comprise a plurality of hexagonal locations.

The array may comprise a plurality of individually addressable locations (e.g., 201). In some instances, the locations may correspond to individually addressable coordinates on the substrate. Alternatively or additionally, the locations may correspond to physical structures (e.g., wells) on the substrate. An analyte to be processed and/or detected in the sequencing system may be immobilized to the array. The array may comprise one or more binders described herein, such as one or more physical linkers or adapters or chemical linkers or adapters that are coupled to, or configured to couple to, an analyte. For instance, the array may comprise a linker or adaptor that is coupled to a nucleic acid molecule. Alternatively or additionally, the analyte may be coupled to a bead (or other support), and the bead (or other support) may be immobilized to the array.

The individually addressable locations may comprise locations of analytes or groups of analytes that are accessible for manipulation. The manipulation may comprise a processing operation by the processing station 104, such as involving placement, extraction, reagent dispensing, seeding, heating, cooling, or agitation. The extraction may comprise extracting individual analytes or groups of analytes. For instance, the extraction may comprise extracting at least 2, at least 5, at least 10, at least 20, at least 50, at least 100, at least 200, at least 500, or at least 1,000 analytes or groups of analytes. Alternatively or additionally, the extraction may comprise extracting at most 1,000, at most 500, at most 200, at most 100, at most 50, at most 20, at most 10, at most 5, or at most 2 analytes or groups of analytes. The manipulation may be accomplished through, for example, localized microfluidic, pipet, optical, laser, acoustic, magnetic, and/or electromagnetic interactions with the analyte or its surroundings in the system.

The array may be coated with binders. For instance, the array may be randomly coated with binders. Alternatively, the array may be coated with binders arranged in a regular pattern (e.g., in linear arrays, radial arrays, hexagonal arrays etc.). The array may be coated with binders on at least 10%, at least 20%, at least 30%, at least 40%, at least 50%, at least 60%, at least 70%, at least 80%, at least 90%, at least 91%, at least 92%, at least 93%, at least 94%, at least 95%, at least 96%, at least 97%, at least 98%, or at least 99% of the number of individually addressable locations, or of the surface area of the substrate. The array may be coated with binders on a fraction of individually addressable locations, or of the surface areas of the substrate, that is within a range defined by any two of the preceding values. The binders may be integral to the array. The binders may be added to the array. For instance, the binders may be added to the array as one or more coating layers on the array.

The binders may immobilize analytes through non-specific interactions, such as one or more of hydrophilic interactions, hydrophobic interactions, electrostatic interactions, physical interactions (for instance, adhesion to pillars or settling within wells), and the like. In some instances, the binders may immobilize biological analytes through specific interactions. For instance, where a biological analyte is a nucleic acid molecule, the binders may comprise oligonucleotide adaptors configured to bind to the nucleic acid molecule. Alternatively or additionally, such as to bind other types of analytes, the binders may comprise one or more of antibodies, oligonucleotides, aptamers, affinity binding proteins, lipids, carbohydrates, and the like. The binders may immobilize biological analytes through any possible combination of interactions. For instance, the binders may immobilize nucleic acid molecules through a combination of physical and chemical interactions, through a combination of protein and nucleic acid interactions, etc. The array may comprise a number of binders on the order of at least about 10, 10², 10³, 10⁴, 10⁵, 10⁶, 10⁷, 10⁸, 10⁹, 10¹⁰, 10¹¹, 10¹², or more. Alternatively or additionally, the array may comprise a number of binders on the order of at most about 10¹², 10¹¹, 10¹⁰, 10⁹, 10⁸, 10⁷, 10⁶, 10⁵, 10⁴, 10³, 10², 10 or fewer binders. The array may have a number of binders that is within a range defined by any two of the preceding values. In some instances, a single binder may bind a single analyte (e.g., nucleic acid molecule). In some instances, a single binder may bind a plurality of analytes (e.g., plurality of nucleic acid molecules). In some instances, a plurality of binders may bind a single analyte. Though some examples herein describe interactions of binders with nucleic acid molecules, the binders may immobilize other molecules (such as proteins), other particles, cells, viruses, other organisms, or the like, and non-biological analytes.

In some instances, each location, or a subset of such locations, may have immobilized thereto an analyte (e.g., a nucleic acid molecule, a protein molecule, a carbohydrate molecule, etc.). An analyte may be immobilized to a location directly or indirectly. For example, an analyte may be immobilized to a location via a particle (e.g., bead) to which it is coupled (e.g., the particle is immobilized to the location and the analyte is coupled to the particle, as described herein). In other instances, a fraction of the plurality of individually addressable location may have immobilized thereto an analyte. For example, at most about 95%, 90%, 85%, 80%, 75%, 70%, 65%, 60%, 55%, 50%, 45%, 40%, 35%, 30%, 25%, 20%, 15%, 10%, 5%, 1%, or fewer individually addressable locations of a substrate or portion thereof may comprise an analyte immobilized thereto. A plurality of analytes immobilized to a substrate may be copies of a template analyte. For example, a plurality of analytes (e.g., nucleic acid molecules) may have sequence homology. A plurality of analytes having sequence homology may comprise a clonal population of nucleic acid molecules (e.g., as described herein). A plurality of analytes having sequence homology may be immobilized to a same location of a substrate (e.g., via a particle, as described herein). Alternatively or additionally, a plurality of analytes having sequence homology may be immobilized to one or more different locations of a substrate. In other instances, a plurality of analytes immobilized to a substrate may not be copies of one another. A plurality of analytes may be of the same type of analyte (e.g., a nucleic acid molecule) or may be a combination of different types of analytes (e.g., nucleic acid molecules, protein molecules, etc.). A plurality of analytes may derive from a same or different sample.

In some instances, the array may comprise a plurality of types of binders, such as to bind different types of analytes. For example, the array may comprise a first type of binders (e.g., oligonucleotides) configured to bind a first type of analyte (e.g., nucleic acid molecules), and a second type of binders (e.g., antibodies) configured to bind a second type of analyte (e.g., proteins), and the like. In another example, the array may comprise a first type of binders (e.g., first type of oligonucleotide molecules) to bind a first type of nucleic acid molecules and a second type of binders (e.g., second type of oligonucleotide molecules) to bind a second type of nucleic acid molecules, and the like. For example, the substrate may be configured to bind different types of analytes in certain fractions or specific locations on the substrate by having the different types of binders in the certain fractions or specific locations on the substrate.

An analyte may be immobilized to the array at a given individually addressable location of the plurality of individually addressable locations. An array may have any number of individually addressable locations. For instance, the array may have at least 1,000, at least 10,000, at least 100,000, at least 200,000, at least 500,000, at least 1,000,000, at least 2,000,000, at least 5,000,000, at least 10,000,000, at least 20,000,000, at least 50,000,000, at least 100,000,000, at least 200,000,000, at least 500,000,000, at least 1,000,000,000, at least 2,000,000,000, at least 5,000,000,000, at least 10,000,000,000, at least 20,000,000,000, at least 50,000,000,000, at least 100,000,000,000, at least 1,000,000,000,000, or more individually addressable locations. The array may have a number of individually addressable locations that is within a range defined by any two of the preceding values. Each individually addressable location may be digitally and/or physically accessible individually (from the plurality of individually addressable locations). For example, each individually addressable location may be located, identified, and/or accessed electronically or digitally for mapping, sensing, associating with a device (e.g., detector, processor, dispenser, etc.), or otherwise processing. Alternatively or additionally, each individually addressable location may be located, identified, and/or accessed physically, such as for physical manipulation or extraction of an analyte, reagent, particle, or other component located at an individually addressable location.

Each individually addressable location may have the general shape or form of a circle, rectangle, hexagon, pit, bump, or any other shape or form. Each individually addressable location may have a first lateral dimension (such as a radius for individually addressable locations having the general shape of a circle or a width for individually addressable locations having the general shape of a rectangle). The first lateral dimension may be at least 1 nanometer (nm), at least 2 nm, at least 5 nm, at least 10 nm, at least 20 nm, at least 50 nm, at least 100 nm, at least 200 nm, at least 500 nm, at least 1,000 nm, at least 2,000 nm, at least 5,000 nm, or at least 10,000 nm. The first lateral dimension may be within a range defined by any two of the preceding values. A lateral dimension may be a cross-sectional dimension such as a diameter. Each individually addressable location may have a second lateral dimension (such as a length for individually addressable locations having the general shape of a rectangle). The second lateral dimension may be at least 1 nanometer (nm), at least 2 nm, at least 5 nm, at least 10 nm, at least 20 nm, at least 50 nm, at least 100 nm, at least 200 nm, at least 500 nm, at least 1,000 nm, at least 2,000 nm, at least 5,000 nm, or at least 10,000 nm. The second lateral dimension may be within a range defined by any two of the preceding values. In some instances, each individually addressable locations may have or be coupled to a binder, as described herein, to couple (e.g., immobilize) an analyte thereto. In some instances, only a fraction of the individually addressable locations may have or be coupled to a binder. For example, at most about 95%, 90%, 85%, 80%, 75%, 70%, 65%, 60%, 55%, 50%, 45%, 40%, 35%, 30%, 25%, 20%, 15%, 10%, 5%, 1%, or fewer individually addressable locations of a substrate or portion thereof may comprise a binder immobilized thereto. In some instances, an individually addressable location may have or be coupled to a plurality of binders to immobilize an analyte thereto.

The analytes associated with the individually addressable locations may include, but are not limited to, molecules, cells, organisms, nucleic acid molecules (e.g., DNA and/or RNA molecules), nucleic acid colonies, particles (e.g., beads), clusters, polonies, and DNA nanoballs. The analytes may be immobilized to the array in a regular, patterned, periodic, random, or pseudo-random configuration, or any other spatial arrangement (e.g., as described herein).

Referring back to FIG. 1, the reagent station 103 may be configured to supply a reagent to the processing station 104. A reagent may comprise any substance, composition, and/or reaction mixture for provision to processing station 104, such as to an analyte, a substrate, and/or an environment of the processing station. A reagent may be useful in the processing of, e.g., an analyte. A reagent may be an enzyme (e.g., polymerase, ligase, nickase, endonuclease, exonuclease, or other enzyme), nucleotide (e.g., as described herein), buffer, cleavage agent, reducing agent, label, detectable material, salt (e.g., magnesium salt such as MgCl₂), stabilizing agent, cryoprotectant, surfactant, binding moiety, or any other useful material. In some instances, a reagent may comprise a nucleotide solution (e.g., comprising one or more different nucleotides), an enzyme solution (e.g., comprising one or more enzymes, as described herein), a wash solution, a buffer solution, a cleavage solution (e.g., comprising a cleavage reagent for cleaving a label from a nucleotide or nucleic acid molecule, or for cleaving or excising a cleavable or excisable base such as a uracil, etc.), water (e.g., deionized water), diluent, and/or any combination thereof. A reagent may comprise a liquid and/or a gas.

The reagent station may comprise and/or be configured to receive one or more reagents. For example, a reagent may be provided in a tube, well, or compartment in the reagent station, or any other container that is capable of isolating a reagent from other reagents. For example, the sequencing system may not include a reagent at a first time, but may include a reagent at a second time (e.g., upon provision by a user). In some instances, for each reagent, at least two reservoirs (e.g., containers) may be provided. The reagent station may be configured to provide the reagent to the processing station from either or all of the at least two reservoirs. Beneficially, when a reagent reservoir is depleted, the other reservoir may be used for continuous supplying to the processing station while the first reservoir is replaced or replenished, without disturbing operations in the processing station. In some instances, each reagent reservoir may be in fluid communication with the processing station. In some instances, reagent volumes from different reservoirs may be dispensed in the processing station through the same outlet. In some instances, reagent volumes from different reservoirs may be dispensed in the processing station through different outlets. In some instances, switching reagent supply from one reservoir to another may comprise manipulating a valve (automatically and/or manually) in fluid connection with each reservoir. In some instances, the reagent station may comprise at least about 1, 2, 3, 4, 5, 6, 7, 8, 9, 10, 20, 30, 40, 50, 60, 70, 80, 90, 100, 200, 300, 400, 500, or more types of reagents. Alternatively or additionally, the reagent station may comprise at most about 500, 400, 300, 200, 100, 90, 80, 70, 60, 50, 40, 30, 20, 10, 9, 8, 7, 6, 5, 4, 3, or 2 types of reagents. Alternatively, the reagent station may comprise a single type of reagent. For example, a reagent station may comprise at least two different types of reagents, such as nucleotides (e.g., of the same or different types) and polymerizing enzymes. In another example, a reagent station may comprise nucleotides, polymerizing enzymes, washing reagents, and cleavage reagents. In some instances, for a reagent, the reagent station may comprise at least about 1, 2, 3, 4, 5, 6, 7, 8, 9, 10, 20, 30, 40, 50, 60, 70, 80, 90, 100, 200, 300, 400, 500, or more reservoirs. Alternatively or additionally, for a reagent, the reagent station may comprise at most about 500, 400, 300, 200, 100, 90, 80, 70, 60, 50, 40, 30, 20, 10, 9, 8, 7, 6, 5, 4, 3, 2, or 1 reservoirs.

In some examples, the process may comprise hot-swapping of reagents, substrates (e.g., wafer) or flow cell, and/or samples in the system. Hot-swapping may comprise switching a part or component of the system such as reagent, substrate, or flow cell without stopping, shutting down, or rebooting the system. In some examples, the method may comprise hot-swapping one or more of reagents, substrate (e.g., wafer), sample, and/or other components of the system. In some examples, the method may comprise hot-swapping all three of the reagents, substrates, and samples. Hot-swapping may offer various advantages such as facilitating 24/7 continuous runs of the systems of the present disclosure. The system may comprise a machine which is configured to draw from multiple system reservoirs. In some cases, hot-swapping may comprise using two or more reservoirs which may be identical. The machine (e.g., drawing machine) may draw the contents of the reservoir one at a time. For example, the machine may draw reagents or samples from the first reservoir until it is nearly empty and then begin to draw from the second reservoir by quickly switching between the two. This ability of the machine to smoothly switch from the first to the second reservoir allows the first reservoir to be replenished or replaced with a new reservoir while the machine continues to function. Alternatively, the machine may draw the contents from multiple reservoirs simultaneously. In some cases, where the system comprises more than two reservoirs, the method may comprise proceeding to draw from the following reservoirs subsequently and respectively. After the machine switches over to the next reservoir, for instance before the second reservoir also runs out, the operator may replenish or swap out the empty first reservoir for a full replacement, in some cases, while the machine is running (e.g., with minimal to no stopping or interruption to the procedure). When the second reservoir runs out, the machine may switch back to drawing from the newly replaced or replenished first reservoir. In some examples, hot-swapping may be performed for the wafers (flow cells) which may be inserted as cartridges and may be hot-swapped according to the methods provided herein to avoid interrupting the workflow of the process. In some examples, samples may be hot-swapped. In some cases, the method may comprise hot-swapping all reagents, wafers, and samples to facilitate un-interrupted extended runs of the sequencer (24 hours per day for several days, weeks, or months). Similarly, any number of additional reservoirs (e.g., third reservoir, fourth reservoir, etc.) may be added to the system for access by one or more machines of the system, while the system is running. Similarly, any number of reservoir not in use may be removed from the system, while the system is running.

In some instances, the reagent station may be configured to automate a reagent thawing operation by regulating one or more conditions of a reagent storage region.

A reagent station may comprise one or more nucleotide solutions. A nucleotide solution may comprise any useful combination of nucleotides. For example, a nucleotide solution may comprise a single type of nucleotide, such as a single type of canonical nucleotide (e.g., adenine, guanosine, uracil, thymine, or cytosine-containing nucleotides). A nucleotide solution may include both labeled (e.g., nucleotides labeled with one or more fluorescent labeling reagents) and unlabeled nucleotides. Labeled and unlabeled nucleotides may be included in any useful proportion. For example, at least about 0.5%, 1%, 1.5%, 2%, 2.5%, 5%, 7.5%, 10%, 15%, 20%, 25%, 30%, 35%, 40%, 45%, 50%, 55%, 60%, 65%, 70%, 75%, 80%, 85%, 90%, 95%, or more nucleotides of a nucleotide solution may be labeled nucleotides. In another example, at most about 0.5%, 1%, 1.5%, 2%, 2.5%, 5%, 7.5%, 10%, 15%, 20%, 25%, 30%, 35%, 40%, 45%, 50%, 55%, 60%, 65%, 70%, 75%, 80%, 85%, 90%, 95%, or fewer nucleotides of a nucleotide solution may be labeled nucleotides. In some cases, all nucleotides of a nucleotide solution may be labeled nucleotides. In other instances, all nucleotides of a nucleotide solution may be unlabeled nucleotides. Nucleotides of a nucleotide solution may be non-terminated nucleotides. Alternatively or additionally, a nucleotide solution may include terminated nucleotides.

In some instances, the reagent station may comprise reagents for each of four or five different types of nucleotides, each of which includes different canonical bases (e.g., adenine (A); cytosine (C); guanine (G); thymine (T); and uracil (U) for thymine (T) when a polynucleotide is ribonucleic acid (RNA)). In some instances, the reagent station may comprise a reagent comprising nucleotides of a single canonical base type. In some instances, the reagent station may comprise a reagent comprising a mixture of nucleotides of multiple canonical base types. A reagent station may include a first nucleotide solution including nucleotides of a first canonical type (e.g., adenine, guanosine, uracil, thymine, or cytosine-containing nucleotides) and a second nucleotide solution including nucleotides of the same canonical type, where at least a fraction of the nucleotides of the first nucleotide solution are labeled and none of the nucleotides of the second nucleotide solution are labeled. The first and second nucleotide solutions may be considered pairs of solutions. A reagent station may include multiple different pairs of nucleotide solutions. For example, a reagent station may include a first pair of nucleotide solutions including adenine-containing nucleotides, a second pair of nucleotide solutions including guanosine-containing nucleotides, a third pair of nucleotide solutions including cytosine-containing nucleotides, and a fourth pair of nucleotide solutions including uracil or thymine-containing nucleotides.

In some instances, the sequencing system 100 may further comprise a diluent station 106 to provide a diluent to, e.g., the processing station 104. In some instances, such diluent can be used to, in real-time, adjust (e.g., increase or decrease) and/or maintain a concentration of a reagent from the reagent station 103 prior to delivery to the processing station. For example, a diluent reservoir and the reagent reservoir of the reagent station may be fluidically connected such that fluids from the reagent reservoir and the diluent reservoir may be merged (e.g., in a pre-determined proportion) prior to the fluids being dispensed or dispersed in the processing station. Merged fluids may be provided in additional reservoirs for storage in advance of use in subsequent processing. Alternatively or additionally, merged fluids may be combined in, e.g., tubes, conduits, or channels that may be configured to provide the merged fluids to the processing station. In some instances, a diluent may comprise water. Water may be, for example, treated water, such as distilled or deionized water. A diluent may comprise a buffer solution. A diluent may comprise any other diluent. The diluent station may comprise one or more diluents. For example, a diluent may be provided in a tube, a well, or compartment in the diluent station, or any other container that is capable of isolating a reagent from other diluents. In some instances, for each diluent, at least two reservoirs (e.g., containers) may be provided. The diluent station may be configured to provide a diluent to the processing station from either or all of the at least two reservoirs. Beneficially, when a diluent reservoir is depleted, the other reservoir may be used for continuous supplying to the processing station while the first reservoir is replaced or replenished, without disturbing operations in the processing station. In some instances, each diluent reservoir may be in fluid communication with the processing station. In some instances, diluent volumes from different reservoirs may be dispensed in the processing station through the same outlet. In some instances, diluent volumes from different reservoirs may be dispensed in the processing station through different outlets. In some instances, switching diluent supply from one reservoir to another may comprise manipulating a valve (automatically and/or manually) in fluid connection with each reservoir. Such a valve may be, for example, a ball valve, butterfly valve, pneumatic valve, gate valve, globe valve, diaphragm valve, plug valve, needle valve, angle valve, pinch valve, slide valve, flush bottom valve, solenoid valve, control valve, flow regulating valve, pressure regulating valve, y-type valve, piston valve, check valve, or any other useful valve. In some instances, the diluent station may comprise at least about 1, 2, 3, 4, 5, 6, 7, 8, 9, 10, 20, 30, 40, 50, 60, 70, 80, 90, 100, 200, 300, 400, 500, or more diluents, which may be of at least about 1, 2, 3, 4, 5, 6, 7, 8, 9, 10, 20, 30, 40, 50, 60, 70, 80, 90, 100, 200, 300, 400, 500 or more different types. Alternatively or additionally, the reagent station may comprise at most about 500, 400, 300, 200, 100, 90, 80, 70, 60, 50, 40, 30, 20, 10, 9, 8, 7, 6, 5, 4, 3, 2, or 1 diluents, which may be of at most about 500, 400, 300, 200, 100, 90, 80, 70, 60, 50, 40, 30, 20, 10, 9, 8, 7, 6, 5, 4, 3, 2, or 1 different types. In some instances, a diluent station may comprise at least about 1, 2, 3, 4, 5, 6, 7, 8, 9, 10, 20, 30, 40, 50, 60, 70, 80, 90, 100, 200, 300, 400, 500, or more reservoirs, which reservoirs may be for 1, 2, 3, 4, 5, 6, 7, 8, 9, 10, 20, 30, 40, 50, 60, 70, 80, 90, 100, 200, 300, 400, 500 or more different diluents (e.g., of the same or different types). Alternatively or additionally, a diluent station may comprise at most about 500, 400, 300, 200, 100, 90, 80, 70, 60, 50, 40, 30, 20, 10, 9, 8, 7, 6, 5, 4, 3, 2, or 1 reservoirs, which reservoirs may be for 1, 2, 3, 4, 5, 6, 7, 8, 9, 10, 20, 30, 40, 50, 60, 70, 80, 90, 100, 200, 300, 400, 500 or more different diluents (e.g., of the same or different types). In some instances, the diluent station may be fluidically connected to a supply of water (e.g., a water storage or a substantially continuous supply of water, such as tap water) to generate filtered and/or deionized water at the system 100 and/or apparatus. For example, the system may comprise a filtration and deionization system for treating water. The filtration and deionization system may comprise one or more filters and/or resins to generate filtered and/or deionized water from the supply of water (e.g., from tap water). Alternatively, the supply of water may not be further filtered or processed prior to mixing with a reagent. In some cases, the methods of the present disclosure may comprise receiving frozen concentrated reagent and performing thawing, dilution, and mixing on the instrument (sequencing system 100). This may have several advantages including reducing the cost and burden of shipping and logistics; for example, because the shipped frozen concentrated reagent may include a decreased amount of water, and in some cases minimal to no water, which makes concentrated reagents easier to transport.

In some cases, two or more reagents (e.g., a nucleotide solution of a first canonical base type and a nucleotide solution of a second canonical base type) may be mixed prior to or during delivery of such reagents to a substrate. For example, as described elsewhere herein, a reagent reservoir may comprise a mixture of reagents, and such mixture may be sourced and dispensed. In another example, respective reagents from different reagent reservoirs may be mixed at an intermediary station or along one or more fluid delivery structures or routes (e.g., channels, etc.), and such mixture sourced and dispensed. Alternatively, two or more reagents may be mixed during or subsequent to their delivery to a substrate. For example, a first reagent solution (e.g., from a first reagent reservoir) may be dispensed on to a region of the surface and a second solution (e.g., from a second reagent reservoir) may be dispensed onto the same or different region of the surface. The first reagent solution and the second solution may come into contact and mixed, for example, at a dispense location (e.g., of both solutions), and/or at a location that they come into contact. The two or more solutions may be dispensed simultaneously or substantially simultaneously. The two or more solutions may be dispensed at different points in time. The two or more solutions may be dispensed form distinct nozzles (e.g., operating units). In some instances, the reagent solutions may be dispensed onto a mixing region of the surface (e.g., radially close to a rotational axis of the surface or a central axis of the surface), which mixing region is substantially lacking samples to which the mixed reagents are configured to reach. For example, the samples may have been dispensed to non-mixing regions (e.g., radially further from the rotational axis of the surface or the central axis of the surface). Subsequent to mixing, the mixture may (e.g., via centripetal, centrifugal or other force due to linear and/or non-linear motion of the surface relative to a reference point) come into contact with the samples in the non-mixing regions. In some embodiments, a solution may be spin-coated onto a surface by dispensing (and/or mixing) the solution at or near the axis of rotation of a rotating substrate such that the centrifugal force of the rotating substrate facilitates the outward spread of the solution away from the axis of rotation. A mixture of reagents, whether generated prior to, during, or subsequent to, delivery to a substrate surface may result in a homogenous solution. In some cases, a substrate may be configured to rotate or otherwise move (e.g., in a linear and/or non-linear motion) relative to a reference point, at constant or variable speed, to facilitate mixing. Such movement parameter may be predetermined, calibrated, adjusted, and/or configured to facilitate mixing.

The processing station 104 may be configured to perform any one or more processing operations in the processing station, such as processing an analyte, processing a substrate, and/or processing an environment of the processing station. For example, processing an analyte may comprise conducting a chemical reaction, biochemical reaction, enzymatic reaction, hybridization reaction, polymerization reaction, physical reaction, any other reaction, or a combination thereof with, in the presence of, or on, the analyte. Processing an analyte may comprise physical and/or chemical manipulation of the analyte. In some instances, processing may involve dispensing or dispersing a reagent to the analyte, the substrate, and/or to the environment of the processing station. The terms “dispense” and “disperse” may be used interchangeably herein. In some cases, dispensing may comprise dispersing and/or dispersing may comprise dispensing. Dispensing generally refers to distributing, depositing, providing, or supplying a reagent, solution, or other object, etc. Dispensing may comprise dispersing, which may generally refer to spreading.

A processing operation may comprise a sequencing operation. A sequencing operation, as used herein, generally refers to an operation performed to generate or identify a sequence of a biological molecule, such as a nucleic acid molecule. Such a sequence may be a nucleic acid sequence, which may include a sequence of nucleotides comprising bases (e.g., adenine, guanosine, uracil, thymine, cytosine, or any other useful bases). Sequencing may comprise single molecule sequencing, sequencing by synthesis, sequencing by hybridization, sequencing by ligation, or any other useful method. Sequencing may be performed using template nucleic acid molecules immobilized on a support, such as a substrate or one or more particles (e.g., beads) (e.g., as described herein).

A processing operation may comprise an amplification operation. The terms “amplifying,” “amplification,” and “nucleic acid amplification” are used interchangeably and generally refer to generating one or more copies of a nucleic acid or a template. For example, “amplification” of DNA generally refers to generating one or more copies of a DNA molecule. An amplicon may be a single-stranded or double-stranded nucleic acid molecule that is generated by an amplification procedure from a starting template nucleic acid molecule. Such an amplification procedure may include one or more cycles of an extension or ligation procedure. The amplicon may comprise a nucleic acid strand, of which at least a portion may be substantially identical or substantially complementary to at least a portion of the starting template. Where the starting template is a double-stranded nucleic acid molecule, an amplicon may comprise a nucleic acid strand that is substantially identical to at least a portion of one strand and is substantially complementary to at least a portion of either strand. The amplicon can be single-stranded or double-stranded irrespective of whether the initial template is single-stranded or double-stranded. Amplification of a nucleic acid may be linear, exponential, or a combination thereof. Amplification may be emulsion based or may be non-emulsion based. Non-limiting examples of nucleic acid amplification methods include reverse transcription, primer extension, polymerase chain reaction (PCR), ligase chain reaction (LCR), helicase-dependent amplification, asymmetric amplification, rolling circle amplification, recombinase polymerase reaction (RPA), and multiple displacement amplification (MDA). Where PCR is used, any form of PCR may be used, with non-limiting examples that include real-time PCR, allele-specific PCR, assembly PCR, asymmetric PCR, digital PCR, emulsion PCR, dial-out PCR, helicase-dependent PCR, nested PCR, hot start PCR, inverse PCR, methylation-specific PCR, miniprimer PCR, multiplex PCR, nested PCR, overlap-extension PCR, thermal asymmetric interlaced PCR and touchdown PCR. Moreover, amplification can be conducted in a reaction mixture comprising various components (e.g., a primer(s), template, nucleotides, a polymerase, buffer components, co-factors, etc.) that participate or facilitate amplification. In some cases, the reaction mixture comprises a buffer that permits context independent incorporation of nucleotides. Non-limiting examples include magnesium-ion, manganese-ion and isocitrate buffers. Additional examples of such buffers are described in Tabor, S. et al. C. C. PNAS, 1989, 86, 4076-4080 and U.S. Pat. Nos. 5,409,811 and 5,674,716, each of which is herein incorporated by reference in its entirety.

Amplification may be clonal amplification. The term “clonal,” as used herein, generally refers to a population of nucleic acids for which a substantial portion (e.g., greater than about 50%, 60%, 70%, 80%, 90%, 95%, or 99%) of its members have sequences that are at least about 50%, 60%, 70%, 80%, 90%, 95%, or 99% identical to one another. Members of a clonal population of nucleic acid molecules may have sequence homology to one another. Such members may have sequence homology to a template nucleic acid molecule. The members of the clonal population may be double stranded or single stranded. Members of a population may not be 100% identical or complementary, e.g., “errors” may occur during the course of synthesis such that a minority of a given population may not have sequence homology with a majority of the population. For example, at least 50% of the members of a population may be substantially identical to each other or to a reference nucleic acid molecule (i.e., a molecule of defined sequence used as a basis for a sequence comparison). At least 60%, at least 70%, at least 80%, at least 90%, at least 95%, at least 99%, or more of the members of a population may be substantially identical to the reference nucleic acid molecule. Two molecules may be considered substantially identical (or homologous) if the percent identity between the two molecules is at least 60%, 70%, 75%, 80%, 85%, 90%, 95%, 98%, 99%, 99.9% or greater. Two molecules may be considered substantially complementary if the percent complementarity between the two molecules is at least 60%, 70%, 75%, 80%, 85%, 90%, 95%, 98%, 99%, 99.9% or greater. A low or insubstantial level of mixing of non-homologous nucleic acids may occur, and thus a clonal population may contain a minority of diverse nucleic acids (e.g., less than 30%, e.g., less than 10%).

Useful methods for clonal amplification from single molecules include rolling circle amplification (RCA) (Lizardi et al., Nat. Genet. 19:225-232 (1998), which is incorporated herein by reference), bridge PCR (Adams and Kron, Method for Performing Amplification of Nucleic Acid with Two Primers Bound to a Single Solid Support, Mosaic Technologies, Inc. (Winter Hill, Mass.); Whitehead Institute for Biomedical Research, Cambridge, Mass., (1997); Adessi et al., Nucl. Acids Res. 28:E87 (2000); Pemov et al., Nucl. Acids Res. 33:e11(2005); or U.S. Pat. No. 5,641,658, each of which is incorporated herein by reference), polony generation (Mitra et al., Proc. Natl. Acad. Sci. USA 100:5926-5931 (2003); Mitra et al., Anal. Biochem. 320:55-65(2003), each of which is incorporated herein by reference), and clonal amplification on beads using emulsions (Dressman et al., Proc. Natl. Acad. Sci. USA 100:8817-8822 (2003), which is incorporated herein by reference) or ligation to bead-based adapter libraries (Brenner et al., Nat. Biotechnol. 18:630-634 (2000); Brenner et al., Proc. Natl. Acad. Sci. USA 97:1665-1670 (2000)); Reinartz, et al., Brief Funct. Genomic Proteomic 1:95-104 (2002), each of which is incorporated herein by reference). The enhanced signal-to-noise ratio provided by clonal amplification more than outweighs the disadvantages of the cyclic sequencing requirement.

A polymerase or polymerizing enzyme may be used in an amplification reaction. The term “polymerizing enzyme” or “polymerase,” as used herein, generally refers to any enzyme capable of catalyzing a polymerization reaction. A polymerizing enzyme may be used to extend a nucleic acid primer paired with a template strand by incorporation of nucleotides or nucleotide analogs. A polymerizing enzyme may add a new strand of DNA by extending the 3′ end of an existing nucleotide chain, adding new nucleotides matched to the template strand one at a time via the creation of phosphodiester bonds. The polymerase used herein can have strand displacement activity or non-strand displacement activity. Examples of polymerases include, without limitation, a nucleic acid polymerase. An example polymerase is a Φ29 DNA polymerase or a derivative thereof. A polymerase can be a polymerization enzyme. In some cases, a transcriptase or a ligase is used (i.e., enzymes which catalyze the formation of a bond). Examples of polymerases include a DNA polymerase, an RNA polymerase, a thermostable polymerase, a wild-type polymerase, a modified polymerase, E. coli DNA polymerase I, T7 DNA polymerase, bacteriophage T4 DNA polymerase Φ29 (phi29) DNA polymerase, Taq polymerase, Tth polymerase, Tli polymerase, Pfu polymerase, Pwo polymerase, VENT polymerase, DEEPVENT polymerase, EX-Taq polymerase, LA-Taq polymerase, Sso polymerase, Poc polymerase, Pab polymerase, Mth polymerase, ES4 polymerase, Tru polymerase, Tac polymerase, Tne polymerase, Tma polymerase, Tea polymerase, Tih polymerase, Tfi polymerase, Platinum Taq polymerases, Tbr polymerase, Tfl polymerase, Pfu-turbo polymerase, Pyrobest polymerase, Pwo polymerase, KOD polymerase, Bst polymerase, Sac polymerase, Klenow fragment, polymerase with 3′ to 5′ exonuclease activity, and variants, modified products and derivatives thereof. In some cases, the polymerase is a single subunit polymerase. The polymerase can have high processivity, namely the capability of the polymerase to consecutively incorporate nucleotides into a nucleic acid template without releasing the nucleic acid template. In some cases, a polymerase is a polymerase modified to accept dideoxynucleotide triphosphates, such as for example, Taq polymerase having a 667Y mutation (see e.g., Tabor et al, PNAS, 1995, 92, 6339-6343, which is herein incorporated by reference in its entirety for all purposes). In some cases, a polymerase is a polymerase having a modified nucleotide binding, which may be useful for nucleic acid sequencing, with non-limiting examples that include ThermoSequenas polymerase (GE Life Sciences), AmpliTaq FS (ThermoFisher) polymerase and Sequencing Pol polymerase (Jena Bioscience). In some cases, the polymerase is genetically engineered to have discrimination against dideoxynucleotides, such as for example, Sequenase DNA polymerase (ThermoFisher).

A polymerase may be Family A polymerase or a Family B DNA polymerase. Family A polymerases include, for example, Taq, Klenow, and Bst polymerases. Family B polymerases include, for example, Vent(exo-) and Therminator polymerases. Family B polymerases are known to accept more varied nucleotide substrates than Family A polymerases. Family A polymerases are used widely in sequencing by synthesis methods, likely due to their high processivity and fidelity.

The term “complementary sequence,” as used herein, generally refers to a sequence that hybridizes to another sequence. Hybridization between two single-stranded nucleic acid molecules may involve the formation of a double-stranded structure that is stable under certain conditions. Two single-stranded polynucleotides may be considered to be hybridized if they are bonded to each other by two or more sequentially adjacent base pairings. A substantial proportion of nucleotides in one strand of a double-stranded structure may undergo Watson-Crick base-pairing with a nucleoside on the other strand. Hybridization may also include the pairing of nucleoside analogs, such as deoxyinosine, nucleosides with 2-aminopurine bases, and the like, that may be employed to reduce the degeneracy of probes, whether or not such pairing involves formation of hydrogen bonds.

In some examples, amplification of samples or portions thereof (e.g., as described herein) may be performed in the processing station 104 of the sequencing system provided herein.

Alternatively or additionally, a separate system may be used to perform processing and/or amplification of the samples prior to their input or loading into system 100 (e.g., to the sample station 101). For example, samples or portions thereof, such as nucleic acid molecules of a sample, may undergo processing and/or amplification prior to their loading onto a substrate. Processing of a sample may comprise, for example, filtration, agitation, centrifugation, storage, transfer, purification, stabilization, selective precipitation, cell lysis, permeabilization, heating, coupling to supports (e.g., particles such as beads, as described herein), primer extension reactions, amplification, ligation, and/or any other useful process. For example, a sample may be prepared by subjecting a plurality of biological and/or biochemical particles of the sample, such as nucleic acid molecules (e.g., DNA, RNA, etc.), to one or more reactions and processes. The plurality of biological and/or biochemical particles (e.g., nucleic acid molecules), and optionally a plurality of supports, such as particles (e.g., beads), may be processed in a pre-processing system (e.g., other than system 100). In the pre-processing system, the biological and/or biochemical particles may be subjected to reactions including amplification reactions such as polymerase chain reactions (PCR). PCR may be performed in compartments such as droplets (e.g., droplets comprising particles such as beads). For example, PCR may comprise or be emulsion PCR (ePCR). For example, the PCR reaction may be performed in a system or medium which may comprise a plurality of partitions or reaction vessels. The partitions or reaction vessels may be microscale or nanoscale. The partitions or reaction vessels may comprise droplets and/or wells. In some examples, a partition may be a droplet in a plurality of droplets in an emulsion (e.g., an aqueous emulsion). For example, an input comprising nucleic acid molecules (e.g., from a sample, as described herein) and particles (e.g., beads) may be compartmentalized in a plurality of droplets in an immiscible phase (e.g., oil) forming an emulsion. The ePCR reaction may be conducted in the droplets. The method may comprise breaking, disrupting, and coalescing the droplets, and extracting or pooling the materials therein, which materials may comprise amplicons (e.g., copies of template nucleic acid molecules, or complements thereof) free in solution and/or coupled to particles (e.g., as described herein).

Where supports (e.g., particles such as beads) are included, amplification reactions (e.g., PCR) may generate nucleic acid molecule (e.g., DNA) colonies coupled to (e.g., immobilized on) the supports (e.g., beads), for example by amplification of the nucleic acid molecules on the supports. The processed supports (e.g., beads) may comprise a plurality of nucleic acid molecules immobilized thereon (e.g., on their surfaces). Such processed supports may, in some examples, be referred to as, e.g., amplified supports (e.g., amplified beads) herein. The processed supports may be pooled and transferred to the sequencing system 100 for input into the sample station 101. The pre-processing system described herein may be separate and independent of the sequencing system 100. Alternatively or additionally, the pre-processing system may be integrated into the sequencing system 100, for example as one or more additional stations. Alternatively or additionally, the pre-processing system may be in operable communication with the sequencing system, or one or more stations thereof. For example, an automated interface, such as an interface comprising a robotic component such as a robotic arm, may automate transfer of materials (e.g., pre-processed samples) between the pre-processing system and the sample station. The sequencing station may receive information on the status of one or more pre-processing operations from the pre-processing station (e.g., via a user interface).

In an example, an analyte may be a nucleic acid molecule from a nucleic acid sample (e.g., as described herein). The nucleic acid molecule may be coupled to (e.g., immobilized to) a substrate (e.g., as described herein, such as a wafer). The nucleic acid molecule may be coupled to the substrate via a support (e.g., particle, such as a bead) to which the nucleic acid molecule is coupled. The processing station 104 may be configured to bring the nucleic acid molecule into contact with one or more reagents for sequencing to identify a sequence of the nucleic acid molecule.

The processing station 104 may be configured to perform a processing operation independent of one or more other operations being performed by, or on, one or more other stations, such as during replacement and/or replenishment of a reagent reservoir in the reagent station 103, during merging of a reagent and a diluent, during substrate loading in the substrate station 102, and/or during sample loading in the sample station 101. The processing station 104 may be configured to perform a processing operation while one or more instructions are updated, such as upon input by a user into a user interface or control system (e.g., as described elsewhere herein). In some instances, the processing station 104 may be configured to operate without human intervention for at least about 1 hour, such as at least about 2, 3, 4, 5, 6, 7, 8, 9, 10, 11, 12, 13, 14, 15, 16, 17, 18, 19, 20, 21, 22, 23, 24, 36, 48, 60, 72 hours, or longer. In some cases, the processing station may be capable of and/or configured to run continuously (e.g., without human intervention) for 24 hours a day, for at least 0.5, 1, 2, 3, 4, 5, 6, 7, 8, 9, 10, or more consecutive days without interruption or human intervention. In some cases, the processing station may be capable of and/or configured to run continuously (e.g., without human intervention) for at least 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, or more weeks without interruption or human intervention. In some cases, the processing station may be capable of and/or configured to run continuously (e.g., without human intervention) for at least 1 month, 2 months, 3 months, 4 months, 5 months, 6 months, 7 months, 8 months, or more months without interruption or human intervention. In some cases, the processing station and/or the sequencing system may not have a start/stop interface (e.g., button, lever, etc.). In other cases, the processing station and/or sequencing station may comprise a start/stop interface (e.g., button, lever, etc.) that may be accessed by a user (e.g., a human operator) to start, pause, or cancel an operation of the system.

In some instances, the processing station 104 may be configured to perform one or more operations of the detection station 105 described elsewhere herein.

The detection station 105 may be configured to perform a detection operation with respect to an analyte, such as an analyte that has undergone processing described herein. In some instances, detecting an analyte may comprise detection of a chemical change or physical change, addition of or subtraction of material, atoms, or molecules, molecular confirmation, detection of the presence of a fluorescent label, detection of a Forster resonance energy transfer (FRET) interaction, or inference of absence of fluorescence. In some instances, the detection station may comprise one or more detector units. A detector unit may comprise a detector. The term “detector,” as used herein, generally refers to a device that is capable of detecting a signal, including a signal indicative of the presence or absence of one or more incorporated nucleotides or fluorescent labels. The detector may detect multiple signals. The signal or multiple signals may be detected in real-time during, substantially during a biological reaction, such as a sequencing reaction (e.g., sequencing during a primer extension reaction), or subsequent to a biological reaction. In some cases, a detector can include optical and/or electronic components that can detect signals. The term “detector” may be used in detection methods. Non-limiting examples of detection methods include optical detection, spectroscopic detection, electrostatic detection, electrochemical detection, acoustic detection, magnetic detection, and the like. Optical detection methods include, but are not limited to, light absorption, ultraviolet-visible (UV-vis) light absorption, infrared light absorption, light scattering, Rayleigh scattering, Raman scattering, surface-enhanced Raman scattering, Mie scattering, fluorescence, luminescence, and phosphorescence. Spectroscopic detection methods include, but are not limited to, mass spectrometry, nuclear magnetic resonance (NMR) spectroscopy, and infrared spectroscopy. Electrostatic detection methods include, but are not limited to, gel-based techniques, such as, for example, gel electrophoresis. Electrochemical detection methods include, but are not limited to, electrochemical detection of amplified product after high-performance liquid chromatography separation of the amplified products.

Detection may comprise continuous area scanning. The term “continuous area scanning,” as used herein, generally refers to area scanning in linear or non-linear paths such as rings, spirals, or arcs on a moving (e.g., rotating and/or translation) substrate using an optical imaging system and a detector. Continuous area scanning may comprise use of an imaging array sensor capable of continuous integration over a scanning area in which the scanning is synchronized (e.g., electronically synchronized) to the image of an object in relative motion. The terms “motion relative to” and similar variations (e.g., “movable relative to,” “moving relative to,” “relative motion,” etc.), as used herein with reference to a relationship between a first object and a second object (e.g., motion of a first object relative to a second object), generally refer to motion by the first object, motion by the second object, or both, relative to the other. For example, relative motion between the detector units and the substrate may refer to motion by the detector units, the substrate, or both.

Continuous area scanning may scan a substrate or array along a nonlinear path. Alternatively or additionally, continuous area scanning may scan a substrate or array along a linear or substantially linear path. The detector may be a continuous area scanning detector. The scanning direction may be substantially θ in an (R, θ) coordinate system in which the object rotation motion is in a θ direction. Across any field of view on the object (substrate) imaged by a scanning system, the apparent velocity may vary with the radial position (R) of the field point on the object as R dθ/dt. Continuous area scanning detectors may scan at the same rate for all image positions and therefore may not be able to operate at the correct scan rate for all imaged points in a curved (or arcuate or non-linear) scan. Therefore, the scan may be corrupted by velocity blur for imaged field points moving at a velocity different than the scan velocity. Continuous rotational area scanning may comprise an optical detection system or method that makes algorithmic, optical, and/or electronic corrections to substantially compensate for this tangential velocity blur, thereby reducing this scanning aberration. For example, the compensation is accomplished algorithmically by using an image processing algorithm that deconvolves differential velocity blur at various image positions corresponding to different radii on the rotating substrate to compensate for differential velocity blur. In some cases, the camera or scanner may apply or use a blur to compensate for differential velocity blur.

In another example, the compensation is accomplished by using an anamorphic magnification gradient. The term “anamorphic magnification”, as used herein, generally refers to differential magnification between two axes of an image. An anamorphic magnification gradient may comprise differential anamorphic magnification in a first axis across a displacement in the second axis. The magnification in the second axis may be unity or any other value that is substantially constant over the field. This may serve to magnify the substrate in one axis (anamorphic magnification) by different amounts at two or more substrate positions transverse to the scan direction. The anamorphic magnification gradient may modify the imaged velocities of the two or more positions to be substantially equal thereby compensating for tangential velocity differences of the two positions on the substrate. This compensation may be adjustable to account for different velocity gradients across the field of view at different radii on the substrate.

The term “field of view”, as used herein, generally refers to the area on the sample or substrate that is optically mapped to the active area of the detector. The imaging field of view may be segmented into two or more regions, each of which can be electronically controlled to scan at a different rate. These rates may be adjusted to the mean projected object velocity within each region. The regions may be optically defined using one or more beam splitters or one or more mirrors. The two or more regions may be directed to two or more detectors. The regions may be defined as segments of a single detector.

The term “continuous area scanning detector,” as used herein, generally refers to an imaging array sensor capable of continuous integration over a scanning area wherein the scanning is electronically synchronized to the image of an object in relative motion. A continuous area scanning detector may comprise a time delay and integration (TDI) charge coupled device (CCD), Hybrid TDI, or complementary metal oxide semiconductor (CMOS), or pseudo TDI device. For example, a continuous area scanning detector may comprise a TDI line-scan camera. Beneficially, relative motion between the one or more detection units in the detection station 105 and the substrate may significantly increase detection efficiency.

Different processing operations on substrates (e.g., open substrates), scanning mechanisms, and optical detection systems are described in International Pub. No. WO 2019/099886 and U.S. application Ser. No. 16/677,115, filed Nov. 7, 2019, which are entirely incorporated herein by reference for all purposes.

In some examples, rotational scanning may be configured to use optics efficiently, for example, more efficiently compared to a static or otherwise non-rotating scanning system. In some cases, latency of a rotating detector unit (e.g., camera) may be decreased or removed when rasterized. In some cases, rotational scanning may decrease or substantially remove the principal latency driven by scan head accelerations. In some cases, scan head accelerations may comprise negative accelerations such as reversal in scan direction. Scan head accelerations may be more likely to occur on linear path scanners compared to the rotational scanning systems provided herein. Stated a different way, rotational scanning may increase the efficiency and throughput of scanning, at least partially due to the decrease in camera latency and/or latency driven by scan head or accelerations thereof. The methods and systems of the present disclosure may comprise a drum scanning system. A detection system may be configured to perform ribbon- or tape-scanning, which may comprise scanning a tape over a drum or roller. Ribbon- or tape-scanning may also comprise scanning a section of a ribbon or tape that is flat (e.g., stretched between two rollers).

Detection may be performed on an object on a substrate. A detector unit may be configured to operate in any useful environment. For example, a detector unit or portion thereof may be configured to operate when the detector unit or portion thereof is at least partially immersed (e.g., submerged) in a fluid. A detector unit or portion thereof may be configured to operate during or after dispensing of a fluid on a substrate, including during spraying of a fluid on a substrate or during removal of a fluid from a substrate, such as via an operation involving a squeegee or other removal mechanism. Additional details of detector systems, including immersion optic systems, are available in, for example, International Patent Publications Nos. WO2019/099886, WO2020/118172, and WO2020/186243, each of which is herein incorporated by reference in their entireties for all purposes.

The power station 108 may comprise an electrical connection to one or more power sources, such as to supply electrical energy to various electrical components of the sequencing system 100, such as, a computer system in the controlling station 107, one or more detector units in the detection station 105, one or more mechanical components (e.g., engines, actuators, valves, etc.) for movement of one or more components of the system (e.g., substrate, detector, etc.), a user interface device of the instructions station 109 (e.g., a monitor), etc. For example, the power source may be a connection to a power grid. Alternatively or additionally, the power source may comprise an energy storage system, such as a battery system (e.g., lithium ion batteries) which may or may not be rechargeable, supercapacitors, ultracapacitors, fuel cells, and the like.

The instructions station 109 may comprise a user interface configured to receive user instructions. The user interface may comprise a graphical user interface (GUI). The user interface may be configured to display a status of one or more stations of the system (e.g., via one or more different displays, such as one or more different screens). The user interface may be configured to display a status of one or more operations in one or more different stations of the system (e.g., via one or more different displays, such as one or more different screens). The user interface may be configured to display a status of one or more components in one or more stations of the system. The user interface may be configured to receive user instructions using any type of user input (e.g., programming input, optical input, audio input, or mechanical input), such as via a keyboard, mouse, voice, touch, and/or any other useful mechanisms. The user interface may also be configured to provide a report relating to one or more stations of the system or one or more operations of one or more stations of the systems. For example, the user interface may be configured to display one or more parameters relating to a sequencing process, including progress of a sequencing reaction (e.g., reagent flows, sequencing cycle numbers, etc.), Phred quality scores, error rates, signal intensities, peak data, information indicative of an orientation of a read (e.g., 5′→3′ designation), etc. The user interface may be configured to display one or more parameters relating to a sample loading, including a concentration of particles and/or nucleic acid molecules or other analytes loaded on a substrate and/or the location of particles and/or nucleic acid molecules or other analytes on a substrate. The user interface may be configured to display concentrations of reagents and/or diluents; a number of substrates included in the system; battery life (where applicable); network (e.g., internet) connectivity; data processing thresholds; data collection progress; sample identifying information including QR codes, barcodes, sources, volumes, contents, origins, or other information; or any other useful information. The user interface may also be configured to allow a user to alter, download, record, save, delete, upload, share, cancel, pause, start, and/or stop any parameter and/or process, including any parameter and/or process described herein. For example, the user interface may be configured to allow a user to upload or download data relating to a sequencing process (e.g., as described herein), such as to or from a cloud-based or other network or to or from a local processor or data storage location.

The controlling station 107 may comprise one or more controllers. The one or more controllers may, individually or collectively, be in operable communication with the one or more other stations. In some instances, a single controller may be configured to control one or more mores stations in the system. In some instances, a plurality of controllers, individually or in combination, may be configured to control one or more stations in the system. In some instances, two or more stations may be in operable communication such as via the one or more controllers. The controller may comprise software and/or hardware (e.g., actuators, motors, valves, etc.) to operably couple the two or more stations. A controller may comprise a computer system, as described elsewhere herein. Alternatively or additionally, a computer system may comprise a controller. The one or more controllers may, individually or collectively, be configured to perform on-board computation on the system 100. Such on-board computing can provide low latency and continuous computing to mitigate the high data rate (e.g., high data images from sequencing by the system 100). A controller may be configured to interface with a user interface (e.g., as described herein).

Provided are methods and systems for on-board and/or nearby server computation. In some examples, data of any format (e.g., signal reads, images, and/or any other form of data mentioned elsewhere herein) may be buffered on disk. Alternatively or additionally, data may be sent directly to the processing unit. For example, data in the form of images may be sent directly to one or more graphic processing unit(s) (GPUs). Such on-board computing methods and systems may facilitate efficient transmission, storage, and/or analysis of high-throughput data which may be generated at a fast pace in the sequencing system of the present disclosure. For example, data may be generated at a rate of at least about 10 Giga-bases per hour (G-bases/hr: 1 Giga bases correspond to data for 1 trillion nucleotide bases), 20 G-bases/hr, 30 G-bases/hr, 40 G-bases/hr, 50 G-bases/hr, 60 G-bases/hr, 70 G-bases/hr, 80 G-bases/hr, 90 G-bases/hr, 100 G-bases/hr, 110 G-bases/hr, 120 G-bases/hr, 130 G-bases/hr, 140 G-bases/hr, 150 G-bases/hr, 160 G-bases/hr, 170 G-bases/hr, 180 G-bases/hr, 190 G-bases/hr, 200 G-bases/hr, 300 G-bases/hr, 400 G-bases/hr, 500 G-bases/hr, 600 G-bases/hr, 700 G-bases/hr, 800 G-bases/hr, 900 G-bases/hr, 1 Tera-bases (T-bases)/hr, 1.5 T-bases/hr, 2 tera-bases/hr, 5 T-bases/hr, 10 T bases/hr, 20 T-bases/hr, 50 T-bases/hr, 100 T-bases/hr, 150 T-bases/hr, 200 T-bases/hr, or more.

In some cases, the throughput of data generation and the size of data may be too high for the images to be stored (e.g., on a memory). Therefore, in some cases, analysis may be performed by low latency, continuous computing or other techniques, in some cases, without intermediate storage of data (e.g., images). In some examples, the method may comprise using a quantitative (e.g., 4-bit discrimination) monochrome sensor to facilitate high-throughput screening.

In some cases, the system may comprise a number of detecting units (e.g., cameras) for use in analyzing a substrate (e.g., wafer). For example, a number of cameras may be configured to scan and/or image one or more substrates at a time. In an example, the number of cameras per substrate may comprise at least 1, 2, 3, 4, 5, 6, 7, 8, 9, 10, or more. A camera line rate may be at least about 100 kilo-lines per second (k-lines/s or kHz), about 150 kHz, about 200 kHz, about 300 kHz, about 400 kHz, about 500 kHz, about 600 kHz, 700 kHz, 800 kHz, 900 kHz, or more.

In some examples, a dimension of a field (e.g., of an imaging field, as described herein) may be at least about 0.1 millimeters (mm), 0.2 mm, 0.3 mm, 0.4 mm, 0.5 mm, 0.6 mm, 0.7 mm, 0.8 mm, 0.9 mm, 1 mm, 1.2 mm, 1.4 mm, 1.6 mm, 1.7 mm, 1.8 mm, 2 mm, 2.2 mm, 2.3 mm, 2.4 mm, 2.6 mm, 2.8 mm, 3 mm, 3.2 mm, 3.4 mm, 3.6 mm, 3.8 mm, 4 mm, 4.2 mm, 4.4 mm, 4.6 mm, 4.8 mm, 5 mm, or more. In some examples, a dimension of a field (e.g., of an imaging field, as described herein) may be at most about 0.1 millimeters (mm), 0.2 mm, 0.3 mm, 0.4 mm, 0.5 mm, 0.6 mm, 0.7 mm, 0.8 mm, 0.9 mm, 1 mm, 1.2 mm, 1.4 mm, 1.6 mm, 1.7 mm, 1.8 mm, 2 mm, 2.2 mm, 2.3 mm, 2.4 mm, 2.6 mm, 2.8 mm, 3 mm, 3.2 mm, 3.4 mm, 3.6 mm, 3.8 mm, 4 mm, 4.2 mm, 4.4 mm, 4.6 mm, 4.8 mm, 5 mm, or less. A dimension of a field may be a width, length, or any other useful dimension. An imaging field may be approximately square or rectangular. An imaging field may correspond to any useful number of pixels. A pixel may be of any useful size. For example, a pixel may have a dimension of about 1 mm. For example, a pixel may be a square having a side length of about 1 mm. For example, a field of view may have a dimension of at least 1, 2, 3, 4, 5, 6, 7, 8, 9, 10, 12, 15, 20, 25, 30, 35, 40, 45, 50, 60, 70, 80, 90, 100, 112, 150, 200, 256, 300, 400, 500, 512, 600, 700, 800, 900, 1000, 1024, 1500, 2000, 2048, or any more pixels. For example, a field of view may have an area of at least about 112×112 pixels², 256×256 pixels², 512×512 pixels², 1024×1024 pixels², 2048×2048 pixels², or greater.

In some examples, when the sample comprises particles (e.g., beads), the pitch between particles (e.g., beads) may be at least about 0.5 micrometers (μm), 1 μm, 1.5 μm, 2 μm, 2.5 μm, 3 μm, 3.5 μm, 4 μm, 4.5 μm, 5 μm, or more. In some examples, the pitch between particles (e.g., beads) may be at most about 1 μm, 1.5 μm, 2 μm, 2.5 μm, 3 μm, 3.5 μm, 4 μm, 4.5 μm, 5 μm, or less.

It will be appreciated that the illustration of FIG. 1 is not restrictive of physical placement of the different stations in the sequencing system 100 and/or apparatus. In some instances, a station may be modular and easily replaceable by, and/or location switched with, another station of the sequencing system. In some instances, a station may be at least partially housed in an individual compartment or housing. In some instances, each station may be included in a separate compartment or housing. In some instances, multiple stations may be at least partially housed in a same compartment or housing. In some instances, a compartment or housing may comprise a door, window, or opening, such as to allow access into the compartment or housing. A compartment or housing may be directly adjacent to another compartment or housing. Alternatively or additionally, a compartment or housing may be separated from another compartment or housing via a door, window, wall, barrier (including an insulated barrier), seal, or any other useful separation. In some instances, compartments or housings of a system may be configured together in a rack-like array. In some instances, one or more compartments or housings of a system may be physically separate from other components or housings of the system. For example, one or more compartments or housings of a system may have no direct physical connection with other compartments or housings of the system. In some instances, a compartment or housing may be movable relative to another component of the sequencing system, such as the remainder of the sequencing system. For example, a compartment or housing may be configured to be linearly pushed out/pulled in drawer form, such as from a rack-like structure.

In some instances, the sequencing system 100 and/or apparatus may be housed in a single compartment or housing structure. For example, the sequencing system and/or apparatus may be housed in a rack-like structure. The sequencing system 100 and/or apparatus may be movable as a single system and/or apparatus, such as by moving the single compartment or housing structure.

Two or more different stations described herein may be combined, such as in a single compartment or housing, and/or one or more operations described herein may be performed by one or more different stations. For example, the processing station may be configured to also perform operation(s) of the detection station, such as detection. For example, the reagent station may be configured to also perform operation(s) of the diluent station, such as supply of a diluent. For example, the controlling station may be configured to also perform operation(s) of the instructions station, and a user interface may be associated with both of these stations.

One or more different stations of a system, or portions thereof, may be subjected to different physical conditions, such as different temperatures, pressures, or atmospheric compositions. In an example, a processing station may comprise a first atmosphere comprising a first set of conditions and a second atmosphere comprising a second set of conditions. The system may comprise a barrier system configured to maintain different physical conditions of one or more different stations of the system, or portions thereof.

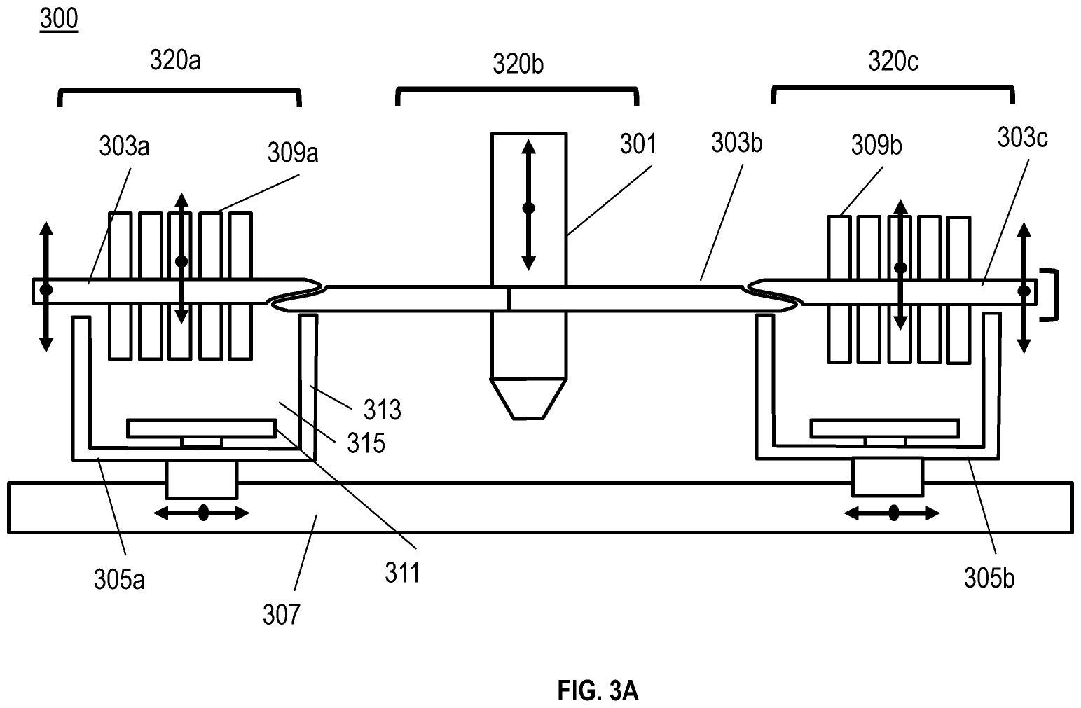

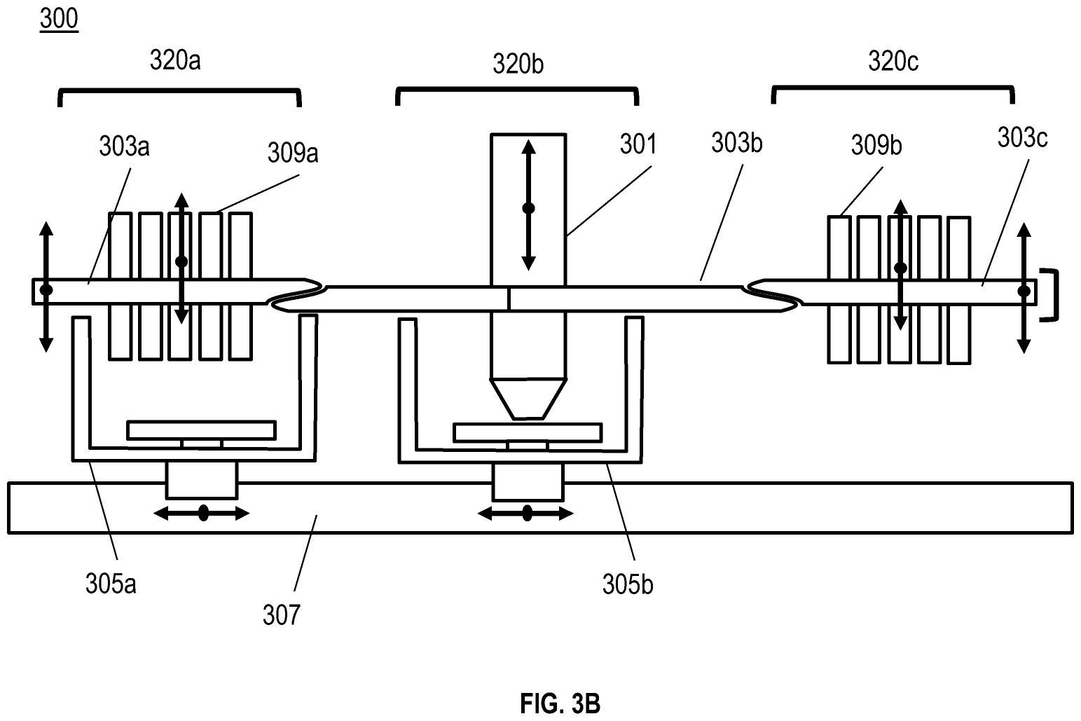

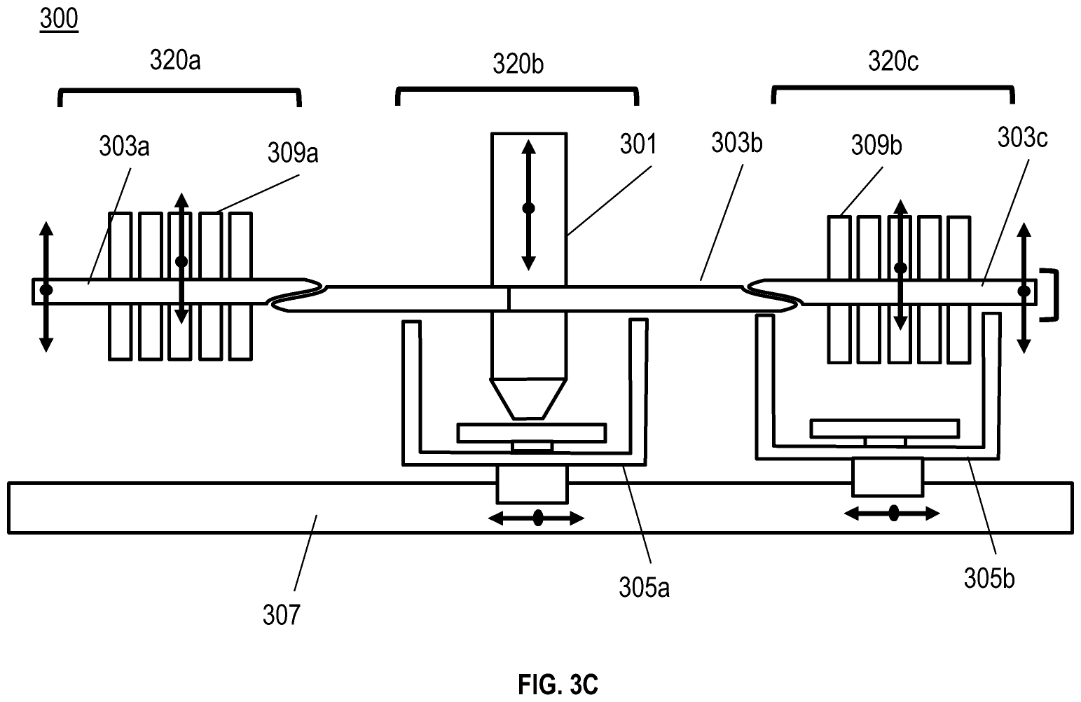

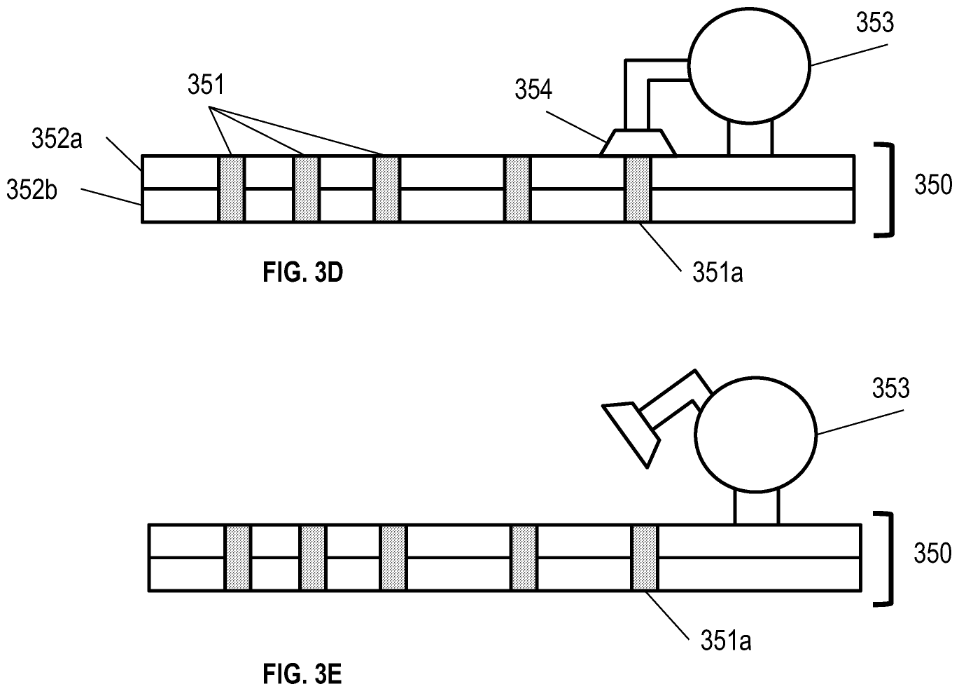

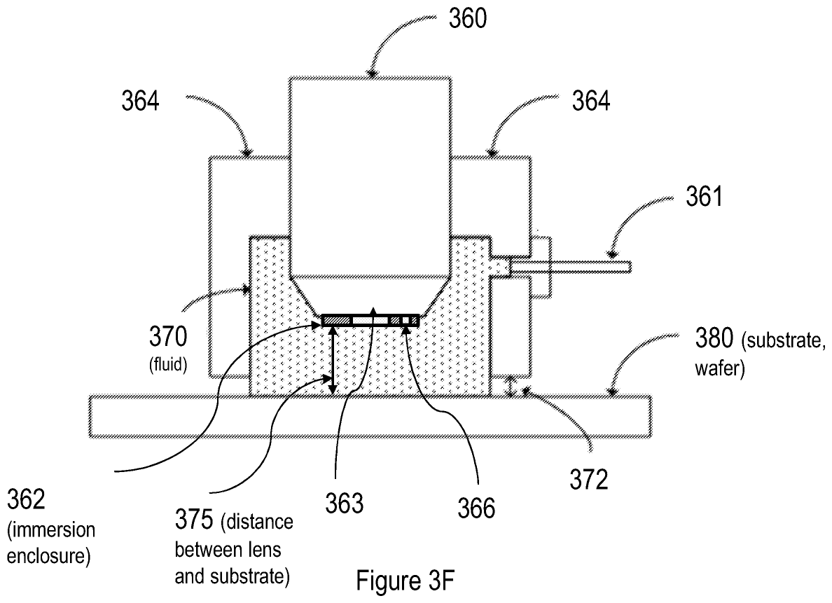

In some instances, the sequencing system 100 may be scaled up to include two or more of a same station type. For example, a sequencing system may include multiple processing and/or detection stations. FIGS. 3A-3C illustrate a part of the sequencing system 300 comprising two processing stations 320a, 320c and a detection station 320b, according to certain embodiments of the present disclosure. In another example, the system 300 may comprise one or more modular sample environment systems (e.g., 305a and 305b in FIGS. 3A-3C).

Referring to FIG. 3A, a modular sample environment system 305a may be configured to receive and/or contain a substrate 311 for processing and/or detection at the processing station and/or detection station, respectively. One or more samples may be immobilized on or adjacent to the substrate. Alternatively or additionally, the one or more samples may otherwise be disposed on the substrate. In some instances, the chamber 313 may be coupled to the substrate. In some instances, the substrate may be fixed relative to the chamber. Alternatively, the substrate may be movable relative to the chamber, for example, in a linear and/or non-linear (e.g., rotational) direction. For example, the substrate may be rotatable relative to the chamber, such as with respect to a rotational axis. The rotational axis may correspond to a central axis of the substrate. The rotational axis may be any axis. The modular sample environment system may be configured to control a sample environment 315 from an external environment. For example, the sample environment may be a controlled environment. The external environment may be an open or closed environment. In some instances, the sample environment may comprise different controlled local environments within the sample environment. The sample environment region may be defined by a chamber 313, a plate 303, and a fluid barrier between the chamber and the plate. The chamber and the plate may be independent such that the chamber, and the sample environment region defined thereby, is movable relative to the plate. The plate and the chamber may not be in direct mechanical contact, such that there is a minimal distance (e.g., in the order of micrometers or millimeters, e.g., at least or at most about 0.1 millimeter (mm), 0.2 mm, 0.3 mm, 0.4 mm, 0.5 mm, 0.6 mm, 0.7 mm, 0.8 mm, 0.9 mm, 1 mm, etc.) between the plate and the chamber. The fluid barrier may comprise fluid from the sample environment, the external environment, or both, and act as a transition region between the sample environment and the external environment. Systems and methods for controlling sample environments are discussed in International Patent Pub. No. WO2020/118172, which is entirely incorporated herein by reference for all purposes.

At the detection station 320b, a detector 301 may protrude into the sample environment (e.g., 315) from the external environment through the plate 303, such as through an aperture in the plate, when a modular sample environment system 305a, 305b is disposed at the detection station. At least a portion of the detector may be fixed relative to the plate. In some instances, the detector may be capable of translating along an axis that is substantially normal to the plane of the plate (e.g., through the aperture) independent of the plate. Within the sample environment, the detector may be configured to detect the one or more samples disposed on the substrate using an immersion optical system, wherein a portion of the detector inside the sample environment, such as an optical imaging objective, is in optical communication with the substrate through a liquid fluid medium. In some instances, the liquid fluid medium may be disposed on a local region of the substrate. Alternatively, the detector may be in optical communication with the substrate without the liquid fluid medium. Optical imaging systems, such as immersion optical systems, are discussed in International Patent Pub. Nos. WO2020/186243 and WO2019/09986, each of which is entirely incorporated herein by reference for all purposes.

FIG. 3F illustrates example components of a detector 360. The detector 360 may correspond to detector 301 as described with respect to FIGS. 3A-3C. The detector 360 may comprise an objective enclosure 364 that is mechanically coupled thereto to facilitate immersion optics. Alternatively, in some instances, the detector 360 and the objective enclosure 364 may be integrated as a single unit.

The objective enclosure 364 (or objective jacket) may comprise or form an immersion enclosure 362 that is configured to contain immersion fluid 370 during detection, to provide a fluid interface between a target surface and the lens 363 (or a window to the lens). Accordingly, the immersion enclosure 362 may define an enclosure volume of immersion fluid 370 surrounding the lens (or window to the lens) at a distal end (the distal end being proximal to the target surface). The objective enclosure 364 may comprise a fluid channel from an inlet 361 to an outlet 366, which inlet directs fluid from external to the objective enclosure to an outlet 366 which opens into the immersion enclosure 362 that surrounds the objective lens 363 (or window to the lens). In some cases, the fluid channel with inlet 361 and outlet 366 conveys immersion fluid 370 into immersion enclosure 362. In some instances, the objective enclosure may comprise a fluid channel with a second inlet and second outlet, which second inlet draws in immersion fluid from the immersion enclosure and directs the immersion fluid to the second outlet exterior to the objective enclosure. In some instances, the objective enclosure 364 may comprise a plurality of fluid channels (with shared or individual inlets/outlets). In some instances, the immersion enclosure may comprise any shape, size, or form as sufficient to retain immersion fluid during detection, where there are one or more openings (e.g., 366) for one or more fluid channels for the immersion fluid (e.g., an opening for each fluid channel and/or for each input, e.g., 361).

In some instances, the objective enclosure 364 may comprise one or more bumper elements, where the one or more bumper elements are configured to protect the lens 363 (or other optical components). The one or more bumper elements may be configured to be more proximal to the target surface than the lens (or other optical components). The differential distance to the target surface between lens and a bumper element of the one or more bumper elements may be on the order of micrometers or millimeters, for example, at least about 50 μm, 100 μm, 150 μm, 200 μm, 250 μm, 300 μm, 350 μm, 400 μm, 450 μm, 500 μm, 550 μm, 600 μm, 650 μm, 700 μm, 750 μm, or more. Alternatively or in addition, the differential distance may be about 750 μm, 700 μm, 650 μm, 600 μm, 550 μm, 500 μm, 450 μm, 400 μm, 350 μm, 300 μm, 250 μm, 200 μm, 150 μm, 100 μm, 50 μm or less. In an example scale, the lens may be about 300 μm from the target surface (e.g., substrate surface) and a bumper element may be about 150 μm from the target surface. The one or more bumper elements may be arranged in any manner to facilitate protection of the lens (or other optical components). For example, two bumper elements may be placed in radial symmetry with respect to the lens (or other optical components). Any number of bumper elements may be provided. Beneficially, when the detector 360 is in relative motion with respect to a target surface (e.g., substrate surface 380), and is misaligned with such target surface, one or more bumper elements may serve to prevent the lens (or other optical components) from colliding into another object (e.g., the substrate surface, or an analyte or other object on the substrate surface or other component of the sequencing system) and becoming damaged, by the one or more bumper elements colliding into such object first (e.g., before the lens would have collided with such an object).

The detector 360 and/or objective enclosure 364 may comprise one or more sensors to facilitate efficient immersion scanning and equipment protection. For example, pressure, distance, and/or positional sensors may be coupled to or integrated at the distal end of the objective enclosure and/or the detector to provide feedback on efficiency and alignment of the objective. A pressure sensor proximal to the one or more bumper elements may provide feedback on alignment. Other sensors may detect a level of immersion fluid in the immersion enclosure. In some cases, optical signals collected by the detector 360 itself may be used to calibrate the detection procedure for more efficient, accurate, and/or precise output.

In some instances, objective enclosure 364 may be configured to maintain a minimal distance 372 between the objective enclosure and the substrate 380. In some instances, the minimal distance serves to avoid contact between the object enclosure 364 and the substrate 380 during movement of the substrate. The minimal distance may be at least about 100 nanometers (nm), at 200 nm, 300 nm, 400 nm, 500 nm, 1 micrometer (μall), 2 μm, 3 μm, 4 μm, 5 μm, 10 μm, 20 μm, 30 μm, 40 μm, 50 μm, 100 μm, 200 μm, 300 μm, 400 μm, 500 μm, 1 millimeter (mm) or more. Alternatively or in addition to, the minimal distance may be at most about 1 mm, 500 μm, 400 μm, 300 μm, 200 μm, 100 μm, 50 μm, 40 μm, 30 μm, 20 μm, 10 μm, 5 μm, 4 μm, 3 μm, 2 μm, 1 μm, 500 nm, 400 nm, 300 nm, 200 nm, 100 nm or less. Alternatively or in addition to, the minimal distance may be within a range defined by any two of the preceding values.

In some instances, one or more sensors of the detector 360 may be configured to detect a distance 375 between lens 363 and the substrate 380. The distance between the lens and the surface may be at least the minimal distance between the objective enclosure 364 and the substrate (e.g., the objective enclosure prevents contact between the lens and the substrate). Operation of the detector 360 may require proximity between the objective lens 363 and the substrate 380. Thus, in some instances, distance 375 may be approximately the minimal distance 372. For example, in some instances, distance 375 may be at least about 100 nanometers (nm), at 200 nm, 300 nm, 400 nm, 500 nm, 1 micrometer (μm), 2 μm, 3 μm, 4 μm, 5 μm, 10 μm, 20 μm, 30 μm, 40 μm, 50 μm, 100 μm, 200 μm, 300 μm, 400 μm, 500 μm, 1 millimeter (mm) or more. Alternatively or in addition to, the minimal distance may be at most about 1 mm, 500 μm, 400 μm, 300 μm, 200 μm, 100 μm, 50 μm, 40 μm, 30 μm, 20 μm, 10 μm, 5 μm, 4 μm, 3 μm, 2 μm, 1 μm, 500 nm, 400 nm, 300 nm, 200 nm, 100 nm or less. Alternatively or in addition to, distance 375 may be within a range defined by any two of the preceding values. In no instance will distance 375 be less than the minimal distance 372.

The processing station 320a may comprise one or more operating units for performance of one or more processing operations (e.g., sequencing operation). For example, any processing station of the present disclosure may have one or more operating unit(s) alternative to or in addition to a detector (e.g., 301). An operating unit may comprise one or more devices or assembly thereof and be configured to facilitate an operation with respect to a sample or the sample environment (or local environment(s) thereof). For example, an operating unit may comprise one or more detectors configured to facilitate detection of a signal or signal change from a sample. In another example, an operating unit may comprise a fluid dispenser (e.g., 309a, 309b) configured to facilitate reagent or fluid dispensing to a sample, such as an independent dispenser for each nucleotide solution of a single canonical base type. In another example, the fluid dispenser may be configured to facilitate sample dispensing to a substrate. For example, the sample may be distributed as a solution of beads (or other solid supports) comprising analytes immobilized thereto onto the substrate (or adjacent thereto). In some cases, the processing station fluid dispenser (e.g., 309a, 309b) may be configured to minimize splashing when dispensing reagents onto the substrate. In some cases, the fluid dispenser may be at a distance from the substrate.

In some cases, the distance of the fluid dispensers from the substrate may comprise about 0.1 μm to about 1,000 μm. In some cases, the distance of the fluid dispensers from the substrate may comprise about 0.1 μm to about 0.2 μm, about 0.1 μm to about 0.5 μm, about 0.1 μm to about 1 μm, about 0.1 μm to about 2 μm, about 0.1 μm to about 5 μm, about 0.1 μm to about 10 μm, about 0.1 μm to about 15 μm, about 0.1 μm to about 20 μm, about 0.1 μm to about 50 μm, about 0.1 μm to about 100 μm, about 0.1 μm to about 1,000 μm, about 0.2 μm to about 0.5 μm, about 0.2 μm to about 1 μm, about 0.2 μm to about 2 μm, about 0.2 μm to about 5 μm, about 0.2 μm to about 10 μm, about 0.2 μm to about 15 μm, about 0.2 μm to about 20 μm, about 0.2 μm to about 50 μm, about 0.2 μm to about 100 μm, about 0.2 μm to about 1,000 μm, about 0.5 μm to about 1 μm, about 0.5 μm to about 2 μm, about 0.5 μm to about 5 μm, about 0.5 μm to about 10 μm, about 0.5 μm to about 15 μm, about 0.5 μm to about 20 μm, about 0.5 μm to about 50 μm, about 0.5 μm to about 100 μm, about 0.5 μm to about 1,000 μm, about 1 μm to about 2 μm, about 1 μm to about 5 μm, about 1 μm to about 10 μm, about 1 μm to about 15 μm, about 1 μm to about 20 μm, about 1 μm to about 50 μm, about 1 μm to about 100 μm, about 1 μm to about 1,000 μm, about 2 μm to about 5 μm, about 2 μm to about 10 μm, about 2 μm to about 15 μm, about 2 μm to about 20 μm, about 2 μm to about 50 μm, about 2 μm to about 100 μm, about 2 μm to about 1,000 μm, about 5 μm to about 10 μm, about 5 μm to about 15 μm, about 5 μm to about 20 μm, about 5 μm to about 50 μm, about 5 μm to about 100 μm, about 5 μm to about 1,000 μm, about 10 μm to about 15 μm, about 10 μm to about 20 μm, about 10 μm to about 50 μm, about 10 μm to about 100 μm, about 10 μm to about 1,000 μm, about 15 μm to about 20 μm, about 15 μm to about 50 μm, about 15 μm to about 100 μm, about 15 μm to about 1,000 μm, about 20 μm to about 50 μm, about 20 μm to about 100 μm, about 20 μm to about 1,000 μm, about 50 μm to about 100 μm, about 50 μm to about 1,000 μm, or about 100 μm to about 1,000 μm. In some cases, the distance of the fluid dispensers from the substrate may comprise about 0.1 μm, about 0.2 μm, about 0.5 μm, about 1 μm, about 2 μm, about 5 μm, about 10 μm, about 15 μm, about 20 μm, about 50 μm, about 100 μm, or about 1,000 μm. In some cases, the distance of the fluid dispensers from the substrate may comprise at least about 0.1 μm, about 0.2 μm, about 0.5 μm, about 1 μm, about 2 μm, about 5 μm, about 10 μm, about 15 μm, about 20 μm, about 50 μm, or about 100 μm. In some cases, the distance of the fluid dispensers from the substrate may comprise at most about 0.2 μm, about 0.5 μm, about 1 μm, about 2 μm, about 5 μm, about 10 μm, about 15 μm, about 20 μm, about 50 μm, about 100 μm, or about 1,000 μm.

An independent dispenser may be provided for sample dispensing. In another example, an operating unit may comprise an environmental unit configured to facilitate environment regulation of a sample environment. In another example, an operating unit may comprise a light source, heat source, or humidity source. In another example, an operating unit may comprise any one or more sensors. A processing station may have multiple operating units, of the same or different types.

An operating unit (e.g., 309a, 309b, 301) may protrude into the sample environment of a modular sample environment system from the external environment through the plate 303, such as through an aperture in the plate. The fit between the operating unit and the aperture may be fluid-tight such that there is no fluid communication through the aperture when the operating unit is fitted through the aperture. Alternatively, an operating unit may not protrude into the sample environment, for example by penetrating at most the depth of the plate. Some operating unit(s) may protrude, and some operating unit(s) may not protrude. In an example, a first operating unit (e.g., detector, e.g., 301) protrudes into the sample environment, and a second operating unit (e.g., reagent dispenser, e.g., 309a) does not protrude into the sample environment. Alternatively or additionally, the aperture may be hermetically or otherwise sealed. Alternatively or additionally, the plate may be integral to the operating unit, or the operating unit may be integral to the plate. Alternatively, the operating unit may be entirely contained in the sample environment, for example, by affixing a non-sample facing end to the plate. In some instances, at least a portion of the operating unit may be fixed relative to the plate. In some instances, the operating unit may be capable of translating along an axis that is substantially normal to the plane of the plate (e.g., through the aperture) independent of the plate. In some instances, at least a portion of the operating unit (e.g., a portion of the operating unit inside the sample environment region) may be capable of moving (e.g., linearly or nonlinearly, such as rotating) independent of the plate.

In some instances, the system 300 may comprise a plurality of modular plates (e.g., 303a, 303b, 303c) that may be coupled or otherwise fastened to each other to create a substantially uninterrupted plate 303. The fit between adjoining modular plates may be fluid-tight such that there is no fluid communication between the modular plates. Alternatively or additionally, the fit may comprise a hermetic seal. Adjoining modular plates (e.g., a first modular plate and a second modular plate) may be coupled via one or more fastening mechanisms. Examples of fastening mechanisms may include, but are not limited to, complementary threading, form-fitting pairs, hooks and loops, latches, threads, screws, staples, clips, clamps, prongs, rings, brads, rubber bands, rivets, grommets, pins, ties, snaps, VELCRO®, adhesives (e.g., glue), tapes, vacuum, seals, magnets, magnetic seals, a combination thereof, or any other types of fastening mechanisms.

In some instances, the first modular plate and the second modular plate can be fastened to each other via complementary fastening units. For example, the first modular plate and the second modular plate can complete a form-fitting pair. The first modular plate can comprise a form-fitting male component and the second modular plate can comprise a form-fitting female component, and/or vice versa. In some instances, an outer diameter of a protrusion-type fastening unit of the first modular plate can be substantially equal to an inner diameter of a depression-type fastening unit of the second modular plate, or vice versa, to form an interference fit. Alternatively or additionally, the two modular plates can comprise other types of complementary units or structures (e.g., hook and loop, latches, snap-ons, buttons, nuts and bolts, magnets, etc.) that can be fastened together. Alternatively or additionally, the two modular plates can be fastened using other fastening mechanisms, such as but not limited to staples, clips, clamps, prongs, rings, brads, rubber bands, rivets, grommets, pins, ties, snaps, VELCRO®, adhesives (e.g., glue), magnets or magnetic fields, tapes, a combination thereof, or any other types of fastening mechanisms.

In some instances, the first modular plate and the second modular plate can be fastened to each other via an intermediary structure. The intermediary structure may be a linker or connector between the first modular plate and the second modular plate. In some instances, the intermediary structure may be fastened to one or both of the first modular plate and the second modular plate through one or more of any of the fastening mechanisms described herein. The intermediary structure may comprise a solid. The intermediary structure may comprise liquid or gas. The intermediary structure may comprise a gel. In some instances, the intermediary structure may be applied as one phase (e.g., liquid) and transform into another phase (e.g., solid) after passage of time such as to achieve the fastening. For example, the intermediary structure may comprise a fluid adhesive that solidifies to achieve the fastening. In some instances, the intermediary structure may be capable of transforming from a first phase to a second phase, such as from liquid to solid or from solid to liquid, upon application of a stimulus (e.g., thermal change, pH change, pressure change, magnetic field, electric field, etc.) to achieve fastening, unfastening, or both. In some instances, the first modular plate and/or the second modular plate may comprise the intermediary structure. For example, the intermediary structure may be integral to the first modular plate and/or the second modular plate. In some instances, the first modular plate and/or the second modular plate, in part or entirely, may be capable of transforming from a first phase to a second phase, such as from liquid to solid or from solid to liquid, upon application of a stimulus (e.g., thermal change, pH change, pressure change, magnetic field, electric field, etc.) to achieve fastening or unfastening (or both) to the other plate. In some instances, one or both of the two modular plates can be cut into or pierced by the other when the two modular plates are fastened together.

The fastening between the first modular plate and the second modular plate can be temporary, such as to allow for subsequent unfastening of the two modular plates without damage (e.g., permanent deformation, disfigurement, etc.) to the two modular plates or with minimal damage. In some instances, the first modular plate may be capable of repeatedly and readily unfastening from the second modular plate and/or from the remainder of the plate 303.

In some instances, a modular plate may be detachable from another modular plate or a remainder of the plate without disturbing one or more sample environments of respective one or more modular sample environment systems that comprise at least a part of the remainder of the plate, such as during an operation by one or more operating units (e.g., reagent dispensing, washing, detecting, etc.). Beneficially, the detachment of a modular plate may allow access to the chamber, such as to load or unload a chamber in the system 300. The detachment of a modular plate may also allow access to an interior of a chamber of a barrier system, such as to load or unload a substrate from the chamber. The detachment of a modular plate may also allow access to one or more operating units coupled to or otherwise associated with the detached modular plate, such as for maintenance, repair, and/or replacement of the one or more operating units. Such detachment may occur while another barrier system carries on with regular operation (e.g., chemical processing operation, detection operation, etc.).

The system 300 may comprise different stations (e.g., 320a, 320b, 320c) capable of parallel operation. A station may be positioned relative to a section of the plate 303. In some instances, a single modular plate may comprise one or more operating units for a station. In some instances, multiple modular plates may comprise one or more operating units for a station. In some instances, a single modular plate may comprise one or more operating units for multiple stations. In some instances, multiple modular plates may comprise one or more operating units for multiple stations. A processing station may comprise a chemical station (e.g., 320a, 320c), such as for sample loading, reagent dispensing, and/or washing. A processing station may comprise a detecting station (e.g., 320b), such as for detection of a signal or signal change. Any modular sample environment system (e.g., 305a, 305b) of the processing system may be capable of traveling between different stations. Alternatively or additionally, the plate 303 may be capable of traveling relative to any modular sample environment system to position a modular sample environment system with respect to a station (e.g., located with respect to a section of the plate). In some instances, a modular sample environment system may be provided a rail or track 307 or other motion path to allow for travel between the different stations. In some instances, different modular sample environment systems may share the same rail or track or other motion path for travel between the different operating systems (e.g., as illustrated in FIGS. 3A-3C). In such cases, the different modular sample environment systems may be configured to move independent of each other on the same rail or track or other motion path or move in unison. In some instances, different modular sample environment systems may move on a dedicated, separate rail or track or other motion path. The motion path may be linear and/or non-linear (e.g., following an arc or curved path). In some instances, the fluid barrier of a modular sample environment system may be maintained during relative motion between the plate 303 and the modular sample environment system, such as during switch of stations. In some cases, the one or more operating units may be capable of movement relative to the plate 303 (such as along an axis normal to the plate) or removal from the plate 303 to allow a modular sample environment system to be positioned with respect to a station. Alternatively, the one or more operating units may not protrude beyond a surface of the plate, or only minimally protrude from the surface of the plate, such as to allow relative movement between the modular sample environment system and different stations.

Beneficially, different operations within the system may be multiplexed with high flexibility and control. For example, as described herein, one or more processing stations may be operated in parallel with one or more detection stations on different substrates in different modular sample environment systems to reduce or eliminate lag between different sequences of operations (e.g., chemistry first, then detection). For example, a processing station may be performing an operation of loading a substrate with beads (e.g., comprising sample analytes immobilized thereto) while a detection station may be performing a detection operation. In another example, a processing station may be dispensing nucleotides and/or washing solution while a detection station may be performing a detection operation. Each station can be optimized for most efficient use. In an example time scheme, a chemistry cycle can take about 45 seconds per cycle and an imaging cycle can take about 30 seconds per cycle. Depending on the desired results, for example, the imaging cycle may comprise scanning of a complete substrate or part(s) of a substrate once, twice, three times, four times, five times, or more times. In some examples, it may take about 55 seconds to scan an entire substrate once. In another example scheme, a chemistry cycle can take about 30 seconds and an imaging cycle can take about 15 seconds per cycle. In another example, an imaging cycle can take at least about 10 seconds, 20 seconds, 30 seconds, 40 seconds, 50 seconds, 60 seconds, 120 seconds, 180 seconds, 240 seconds, 300 seconds, or more. Alternatively or in addition, an imaging cycle can take at most about 300 seconds, 240 seconds, 180 seconds, 120 seconds, 60 seconds, 50 seconds, 40 seconds, 30 seconds, 20 seconds, 10 seconds, or less. The modular sample environment systems may be translated between the different stations accordingly to optimize efficient equipment use (e.g., such that the detection station is in operation almost 100% of the time). In some examples, at least 1, 2, 3, 4, 5, 6, 7, 8, 9, or more modules or stations of the sequencing system may be multiplexed. For example, 2 or more of the modules may each perform their intended function simultaneously or according to the methods described elsewhere herein. An example of this may comprise two-station multiplexing of an optics station and a chemistry station as described herein. Another example may comprise multiplexing three or more stations and process phases. For example, the method may comprise using staggered chemistry phases sharing a scanning station. The scanning station may be a high-speed scanning station. The modules or stations may be multiplexed using various sequences and configurations.

FIGS. 3A-3C illustrate multiplexing of two sample environment systems in a three-station system. In FIG. 3B, the first chemistry station (e.g., 320a) can operate (e.g., dispense reagents, e.g., to incorporate nucleotides to perform sequencing by synthesis) on a first substrate in a first sample environment system (e.g., 305a) while substantially simultaneously, a detection station (e.g., 320b) can operate (e.g., scan) on a second substrate in a second sample environment system (e.g., 305b), while substantially simultaneously, a second chemistry station (e.g., 320c) sits idle. An idle station may not operate on a substrate. An idle station (e.g., 320c) may be recharged, reloaded, replaced, cleaned, washed (e.g., to flush reagents), calibrated, reset, kept active (e.g., power on), and/or otherwise maintained during an idle time. After an operating cycle is complete, the sample environment systems may be re-stationed, as in FIG. 3C, where the second substrate in the second sample environment system (e.g., 305b) is re-stationed from the detection station (e.g., 320b) to the second chemistry station (e.g., 320c) for operation (e.g., dispensing of reagents, e.g., to incorporate nucleotides to perform sequencing by synthesis) by the second chemistry station, and the first substrate in the first sample environment system (e.g., 305a) is re-stationed from the first chemistry station (e.g., 320a) to the detection station (e.g., 320b) for operation (e.g., scanning) by the detection station. An operating cycle may be deemed complete when operation at each active, parallel station is complete. During re-stationing, the different sample environment systems may be physically moved (e.g., along the same track or dedicated tracks) to the different stations and/or the different stations may be physically moved to the different sample environment systems. After the next operating cycle is complete, the sample environment systems can be re-stationed again, such as back to the configuration of FIG. 3B, and this re-stationing can be repeated (e.g., between the configurations of FIGS. 3B and 3C) with each completion of an operating cycle until the required processing for a substrate is completed. In this illustrative re-stationing scheme, the detection station may be kept active (e.g., not have idle time not operating on a substrate) for all operating cycles by providing alternating different sample environment systems to the detection station for each consecutive operating cycle. Beneficially, use of the detection station is optimized. Based on different processing or equipment needs, an operator may opt to run the two chemistry stations (e.g., 320a, 320c) substantially simultaneously while the detection station (e.g., 320b) is kept idle, such as illustrated in FIG. 3A.

FIGS. 3D-3E illustrate components of sample environment systems, such as described with respect to FIGS. 3A-3C. Plate 350 may correspond to plate 303 or modular plates (e.g., 303a, 303b, 303c) as described with respect to FIGS. 3A-3C. The plate 350 (shown as a side view) may comprise one or more layers (e.g., 352a, 352b). A plurality of layers may be adjacent and stacked to form the plate 350. Such plurality of layers may facilitate insulation of the interior of the sample environment from the external environment, and also allow for customization of one or more layers while maintaining insulation through the other layers. For example, a first layer 352a (top layer) may comprise foam and/or other insulative material and a second layer 352b (bottom layer) may comprise foam and/or other insulative material. The insulative material may seal moisture and temperature (or range thereof) within the sample environment.

The plate 350 may comprise one or more apertures 351 that extend through the depth of plate 350 with an opening to the sample environment. In some cases, an aperture may extend through the depth of one or more layers of a plate with an opening to the sample environment. The one or more apertures may provide access to the sample environment from an exterior environment, such as by the operating units (e.g., 309a, 309b, 301). In some cases, the one or more apertures may otherwise provide access to an object (e.g., sample, reagents, sensors, etc.) inside the sample environment. For example, an object may be placed inside or removed from the sample environment through one or more apertures. An aperture may have an open state and a closed state. FIG. 3D illustrates an aperture 351a in a closed state and FIG. 3E illustrates the aperture 351a in an open state. In a closed state, the aperture may be sealed (e.g., hermetically sealed) to seal the sample environment. In an open state, the aperture may permit access into or out of the sample environment through the aperture. An actuation unit 353 may be provided to alternate the aperture between the open state and the closed state. For example, the actuation unit 353 may comprise a mechanical arm, which comprises a suction cup or other sealing device 354 at one end, which sealing device may be configured to cover or uncover the aperture to close and open the aperture, respectively, by moving the mechanical arm. The actuation unit may have any practical form, such as a sliding or rotary cover disposed in proximity to each aperture, or any other form. A single actuation unit may be capable of and/or configured to control the respective open/close states of multiple apertures simultaneously or at different points in time. For example, a single actuation unit may comprise multiple sealing devices that can approach a plurality of apertures, and/or a single actuation unit may comprise a sealing device that can simultaneously cover a plurality of apertures. Alternatively or in addition, multiple actuation units may be provided to control the respective open/close states of multiple apertures.

In some cases, one or more layers may comprise a channel, track, or other path to facilitate access to an aperture by an operating unit or other object transitioning through the aperture. The channel, track, or other path may be integrated (e.g., as a recess or cut-out) in the one or more layers. The other layer(s) may provide insulation for the sample environment where there is a channel, track, or other path in another layer. For example, a sample nozzle channel can be provided in a top layer (e.g., first layer 352a) of the plate 350 to allow a sample nozzle to access the aperture to penetrate the plate. In another example, a fluidic channel or manifold can be provided in a layer to allow reagents to access the aperture through the fluidic channel or manifold. In some cases, the aperture may start at this layer with the channel and open into the sample environment.

In operation, as an example, after a sample environment is loaded with a substrate, an actuation unit opens aperture 351a, a mechanical arm positions the sample nozzle at aperture 351a by traveling through or moving within the sample nozzle channel in first layer 352a, the sample nozzle dispenses a solution comprising a plurality of beads comprising sample analytes immobilized thereto onto the substrate, the sample nozzle is removed from the aperture, and the actuation unit closes the aperture 351a to seal the sample environment. In another example, after a sample or reagents have been dispensed on a substrate, an actuation unit opens an aperture, a mechanical arm delivers an interferometer into the sample environment to position the sensor for accurate measurement of fluid layer thickness on the substrate, the actuation unit closes the aperture, the interferometer collects signals to determine fluid layer thickness, the actuation unit opens an aperture, the interferometer is removed from the sample environment (e.g., by a mechanical arm), and the actuation unit closes the aperture. It will be appreciated that an object need not enter and exit the sample environment through the same aperture, though it may. Beneficially, any aperture may be opened only at times where access is needed and closed at other times. Accordingly, plate 350 may comprise a plurality of apertures 351 in one or more strategic locations (e.g., with respect to a sample environment, or component therein, such as substrate) that may be sealed, opened, and used as needed based on different operations.

In some instances, the sample nozzle is maintained at about a first height from the substrate while dispensing a solution comprising a plurality of beads onto the substrate (e.g., beads comprising sample analytes immobilized thereto). In some instances, a first height is between about 100 μm and 800 μm, between about 100 μm and 700 μm, between about 100 μm and 600 μm, between about 100 μm and 500 μm, between about 100 μm and 400 μm, between about 100 μm and 300 μm, between about 100 μm and 200 μm, between about 200 μm and 800 μm, between about 200 μm and 700 μm, between about 200 μm and 600 μm, between about 200 μm and 500 μm, between about 200 μm and 400 μm, between about 200 μm and 300 μm, between about 300 μm and 800 μm, between about 300 μm and 700 μm, between about 300 μm and 600 μm, between about 300 μm and 500 μm, between about 300 μm and 400 μm, between about 400 μm and 800 μm, between about 400 μm and 700 μm, between about 400 μm and 600 μm, between about 400 μm and 500 μm, between about 500 μm and 800 μm, between about 500 μm and 700 μm, between about 500 μm and 600 μm, between about 600 μm and 800 μm, between about 600 μm and 700 μm, or between about 700 μm and 800 μm. In some instances, the first height is about 100 μm, 150 μm, about 200 μm, about 250 μm, about 300 μm, about 350 μm, about 400 μm, about 450 μm, about 500 μm, about 550 μm, about 600 μm, about 650 μm, about 700 μm, about 750 μm, about 800 μm or about 850 μm. In some instances, the sample nozzle is maintained at the first height from the substrate within about a first standard deviation while dispensing a solution comprising a plurality of beads onto the substrate (e.g., beads comprising sample analytes immobilized thereto). In some instances, the first standard deviation is about 1 μm, about 2 μm, about 3 μm about 4 μm about 5 μm, about 6 μm, about 7 μm, about 8 μm, about 9 μm, about 10 μm, about 11 μm, about 12 μm, about 13 μm, about 14 μm, about 15 μm, about 16 μm, about 17 μm, about 18 μm, about 19 μm, about 20 μm, about 21 μm, about 22 μm, about 23 μm, about 24 μm, about 25 μm, about 26 μm, about 27 μm, about 28 μm, about 29 μm, about 30 μm, about 31 μm, about 32 μm, about 33 μm, about 34 μm, about 35 μm, about 40 μm, about 45 μm, about 50 μm, about 55 μm, or about 60 μm. In some instances, the first standard deviation is between about 1 μm and 30 μm, between about 5 μm and 30 μm, between about 10 μm and 30 μm, between about 20 μm and 30 μm, between about 1 μm and 25 μm, between about 1 and 20 μm, between about 1 μm and 15 μm, between about 1 μm and 10 μm, between about 1 μm and 5 μm, between about 5 μm and 20 μm, between about 5 μm and 15 μm, between about 5 μm and 10 μm, between about 10 μm and 20 μm, between about 10 μm and 15 μm, or between about 20 μm and 30 μm.

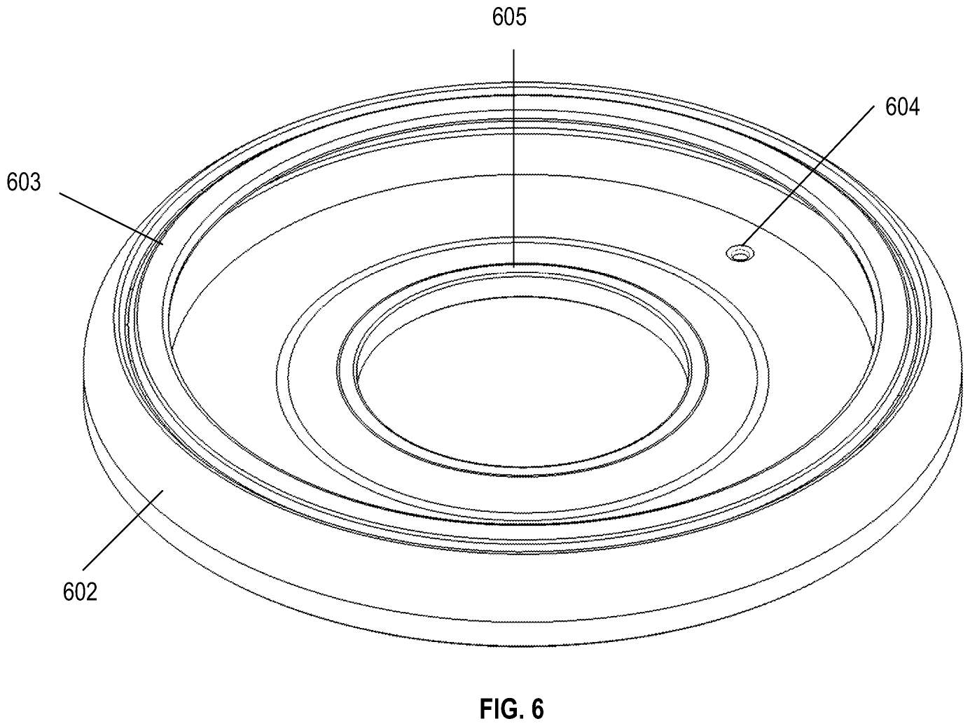

In some instances, the modular sample environment may be a bowl 602, as shown in FIG. 6. The bowl may comprise a circular or arbitrary polygonal shape with a lip or edge 603 configured to prevent liquid from spilling over the edge of the bowl. In some cases, the bowl may comprise one or more drains 604 in fluid communication with a fluidic drain assembly, described elsewhere herein, to evacuate or drain fluid dispensed by the processing stations described elsewhere herein. In some instances, the bowl may comprise a material with anti-corrosive properties. In some cases, the bowl may comprise cut-out feature 605 such that a rotational motor positioned underneath the bowl 602 may be in mechanical communication with a substrate. In some cases, the cut-out feature 605 may be circular or an arbitrary polygonal shape. Bowl 602 is made of material that can withstand the constantly high humidity and highly corrosive environment. In some embodiments, one or more anti-corrosive coatings may be applied to bowl 602.

In some cases, a bowl 602 may comprise a lip or edge 603 that may comprise one or more cut-out regions. In some instances, a cut-out of a lip or edge of the bowl may serve to accommodate the objective enclosure and also to prevent liquid from splashing outside of the bowl (e.g., where splashed liquid may disadvantageously contribute to bowl flooding, as described elsewhere herein). In some cases, the one or more cut-out regions each may comprise a circular or arbitrary polygonal shape. A cut-out may be any arbitrary shape, including non-polygonal shapes. A cut-out may allow for the objective enclosure 364 and the plurality of fluid channels of the objective enclosure (e.g., 361, 366), described elsewhere herein, to image the substrate 380, while preventing liquid from splashing from the substrate to the surrounding modular sample environment.

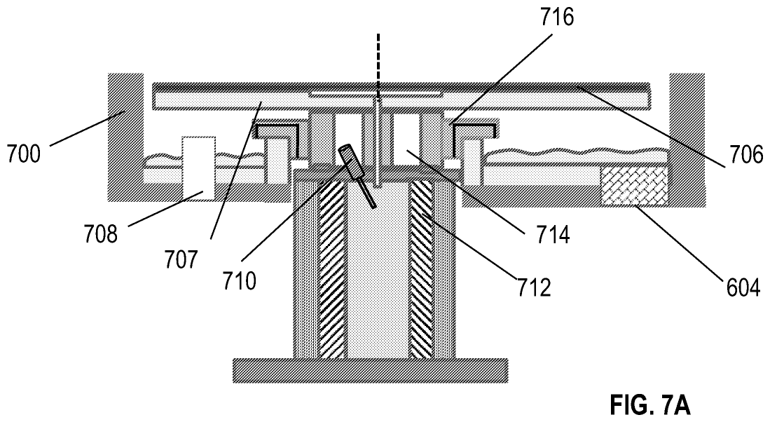

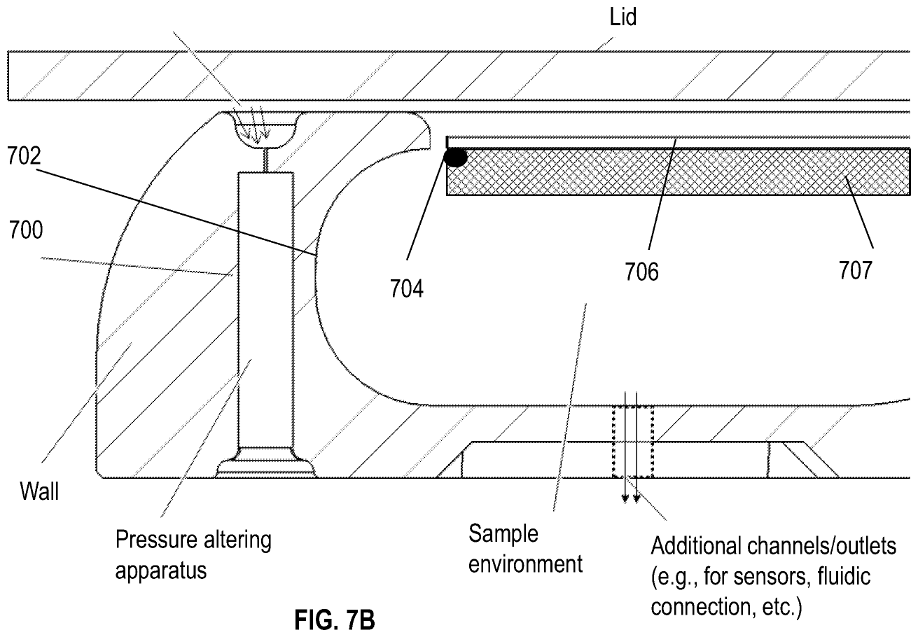

In some instances, a bowl 700 may comprise an internal contour (e.g., 702), as seen in FIGS. 7A-7B, configured to minimize the amount of liquid dispensed onto the substrate that splashes onto a chemical reaction surface and/or escapes the modular sample environment. In some cases, the internal contour may comprise any or a combination of: two linear profiles (e.g., 702), a circular profile with no linear segment, a circular profile with a linear segment, a single linear profile spanning more than half of the height of bowl, or a single linear profile spanning less than half of the height of the bowl.

In some cases, the bowl 700 may comprise one or more liquid sensors 708. In some instances, the one or more liquid sensors 708 may be in electrical communication with one or more processors of the sequencing system, as described elsewhere herein. In some cases, the one or more liquid sensors 708 may be configured to detect a flooding event (e.g., a filling of the bowl above a predetermined threshold level). In some instances, a flooding event is due to a clog and/or improper draining of the bowl.

In some cases, a seal 716 may be positioned such that it is in mechanical communication with the cut-out feature 605 (i.e., the cut-out features for the rotational motor). In some instances, the seal 716 may be made of a plastic, polymer, and/or a rubber material. The seal may be made of polytetrafluoroethylene (PTFE), silicon, fluorinated ethylene propylene (FEP), or any combination thereof. In some instances, the seal 716 may be in mechanical communication with a rotor 714 of a motor 712 permit rotation of the motor while providing a liquid seal between the fluid draining out of the bowl and the electrical components of the motor (e.g., preventing draining fluid from interacting or coming into contact with the electrical components of the motor). In some cases, the rotor 714 of the motor may comprise a metallic material e.g., stainless steel, copper, aluminum, or any combination thereof. In some instances, the rotor 714 of the motor may be in mechanical communication with a chuck 707 to rotate the chuck as the rotor 714 rotates. In some cases, the rotor 714 may comprise one or more through-hole features configured to transmit near-infrared energy to and from a near-infrared source and detector 710. In some cases, the near-infrared source and detector 710 may be configured to determine a temperature of the chuck 707 and/or substrate 706. In some instances, the chuck 707 may comprise a structural enhancement feature 704. The structural enhancement feature 704 may comprise a circular gasket, ceramic ring, or any combination thereof. The structural enhancement feature 704 may assist in maintaining the substrate 706 flat and/or sealing the substrate 706 to the chuck 707. In some instances, the structural enhancement feature 704 may assist in maintaining substrate 706 in a substantially flat (e.g., horizontal) configuration. In some instances, the structural enhancement feature 704 may assist in maintaining substrate 706 in a configuration that is substantially parallel to chuck 707 (e.g., to a face of the chuck). In some instances, the structural enhancement feature 704 may assist in sealing substrate 706 to chuck 707.

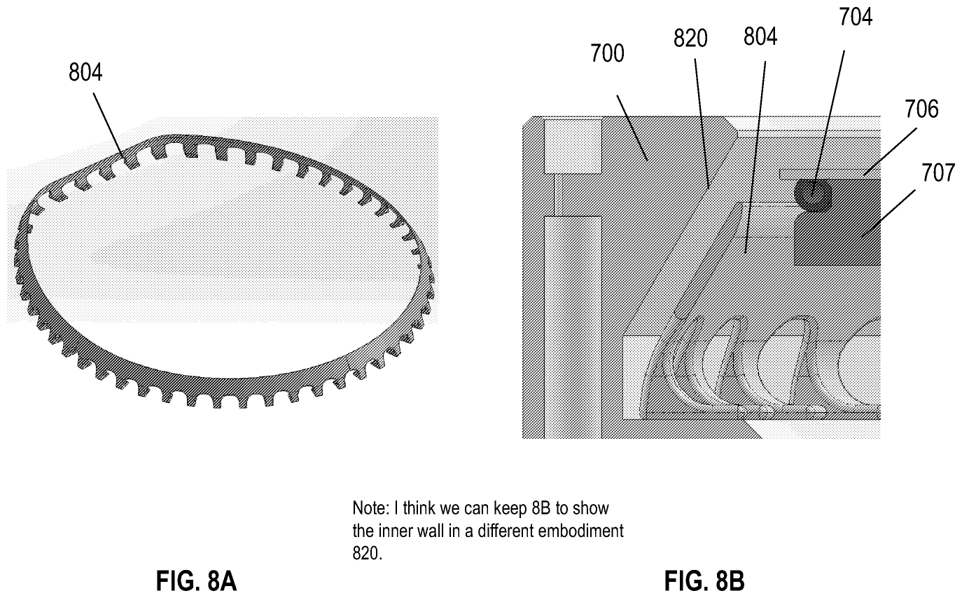

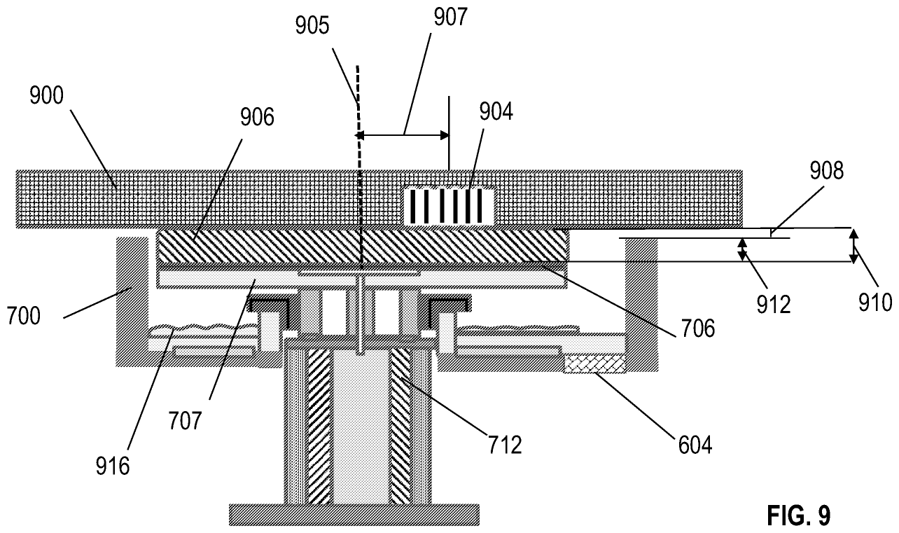

In some cases, the bowl 700 may further comprise a liquid-catching structure 804, as seen in FIGS. 8A-8B that may be placed within the bowl 700 and is configured to absorb liquid that might otherwise be deflected out of the bowl from the substrate 706, structural enhancement feature 704, chuck 707, or any combination thereof. In some cases, the liquid-catching structure 804 may comprise a hydrophilic surface and/or wettable surface. In some cases, the liquid-catching structure 804 may be composed of titanium. In some cases, the liquid-catching structure 804 may comprise titanium, stainless steel, tungsten carbide, or any combination thereof. In some cases, the liquid-catching structure may comprise a corrosion-resistant material. In some instances, the liquid-catching structure 804 may be configured to further assist the lip of the bowl in preventing liquid being deflected out of the bowl. In some cases, the structure of the liquid-catching structure 804 may comprise a plurality of curved geometrical features as seen in FIGS. 8A-8B.

In some cases, the bowl 700 may comprise a modular liquid-catching structure that may be deployed in an open or closed state. For example, in some cases, the modular liquid-catching structure may comprise a uniform or substantially uniform structure without the curved geometrical features such as those of 804. Alternatively, in some cases, the modular liquid-catching structure may comprise one or more geometrical features (e.g., curved or not curved). In some instances, such a modular liquid-catching structure may be an alternative to liquid-catching structure 804, as shown in FIGS. 8A-8B. In some instances, such a modular liquid-catching structure is placed between the chuck 707 and/or substrate 706 and a wall of the bowl 700. In some instances, a bowl 700 comprising such a modular liquid-catching structure may also comprise internal contour 820 (e.g., instead of or in addition to internal contour(s) 702). In some instances, a modular liquid-catching structure may be shaped so as to fit within bowl 700 (e.g., to conform to any internal contours of the bowl). In some cases, the modular liquid-catching structure may be placed within the bowl 700 and be configured to absorb liquid that might otherwise be deflected out of the bowl from the substrate 706, structural enhancement feature 704, chuck 707, or any combination thereof. In some cases, the modular liquid-catching structure may be deployed in an open state with a clearance gap between the top plane of the modular liquid-catching structure and the objective enclosure 364. In some instances, the modular liquid-catching structure may be deployed in a closed state, where the modular liquid-catching structure is folded away from the chuck 707, substrate 706, and structural enhancement feature 704 assembly. In some cases, the modular liquid-catching structure open and/or closed state (e.g., switching between the open state and the closed state) may be controlled by one or more servo motors that are in mechanical communication with the modular liquid-catching structure. In some cases, the one or more servo motors may be in electrical communication with the sequencing system processor, as described elsewhere herein.

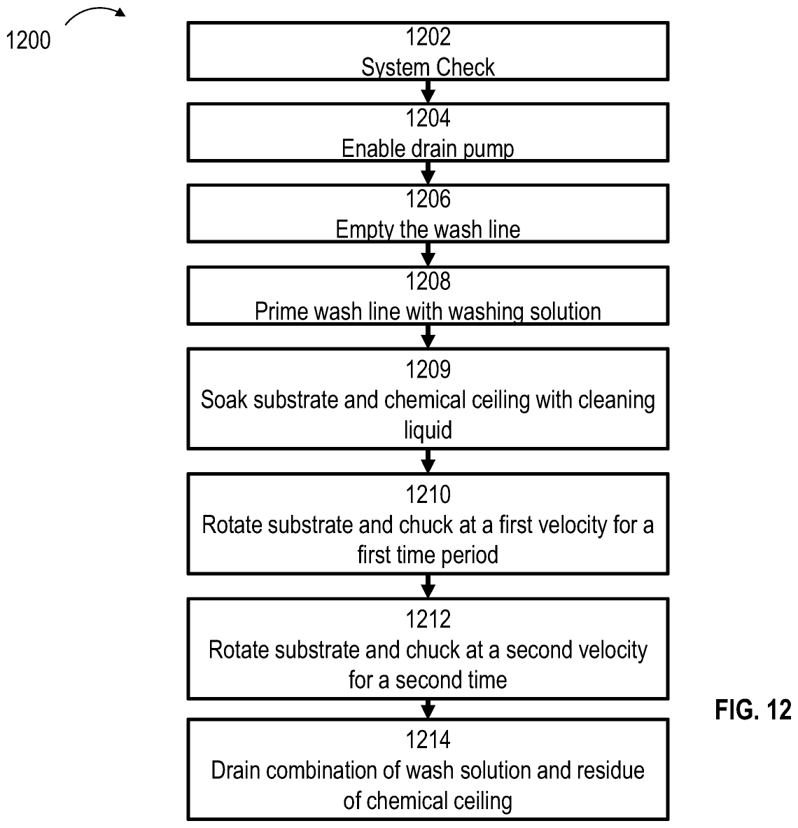

In some instances, residue (e.g., salt residues) may form and/or deposit on a chemical ceiling 900. In some instances, the sequencing systems described herein may comprise a method of cleaning and/or removing residue that has formed and/or deposited on a chemical ceiling 900. The residue that has formed and/or deposited on the chemical ceiling after may be removed. In some cases, the method of cleaning and/or removing residue deposited on a chemical ceiling 900 may comprise one or more of the following operations detailed in process flow 1200 of FIG. 12: (a) performing a sequencing system check 1202; (b) enabling (e.g., initiating) a drain pump configured to drain the liquid collected by the bowl 1204; (c) emptying the fluid previously in fluid communication with the fluid dispenser by flowing the washing solution through the fluid dispenser 1206; (d) preparing a wash solution fluid reservoir to be in fluid communication with the fluid dispenser 1208; (e) dispensing washing solution 1106 by fluid dispensers 904 in a space between the substrate 706 and chemical ceiling (900) 1209; (f) rotating the substrate 706 in mechanical communication with a chuck 707 and/or motor 712 at a first velocity for a first period of time to cause the washing solution 1106 to clean and/or remove residue deposited on the chemical ceiling (900) 1210; (g) rotating the substrate 706 in mechanical communication with the chuck 707 and/or motor 712 at a second velocity for a second period of time 1212; and (h) draining a combination solution of liquid and residue removed from the chemical ceiling 1214.

In some cases, the washing solution may comprise deionized water. In some instances, the washing solution may comprise distilled water. In some cases, the washing solution may comprise nuclease-free (e.g., DNase-free) water. In some instances, the washing solution may comprise any combination of deionized water, distilled water, and nuclease-free water. In some cases, the first velocity may be configured to clean and/or remove residue formed or deposited on the chemical ceiling. In some instances, a second velocity may be configured to displace the washing solution 1106 away from the substrate 706 and chemical ceiling 900. In some cases, the fluid dispensers 904 may be a distance 907 from the center 905 of the substrate 706. In some cases, draining may comprise an active drain where a negative pressure may be applied in fluid communication with the drain 604 to remove liquid from the bowl 700. In some cases, the fluid may be deposited by the fluid dispensers 904, described elsewhere herein. In some instances, the method may further include the operation of allowing the washing solution to soak the chemical ceiling and/or the substrate for a soak duration. In some cases, the residue deposited may comprise a salt residue. In some cases, one or more of operations (e) to (h) may be repeated for 2 or more cycles. In some cases, the washing solution 1106 may have a thickness 910 (e.g., depth) that may comprise the combination of a substrate to bowl distance 912 and a bowl to chemical ceiling distance 908 (e.g., 910 equals the sum of 908 and 912). In some instances, the thickness (e.g., depth 910) of the washing solution 1106 may be maintained by surface tension between the washing solution, substrate and/or the chemical ceiling. In some instances, the total of the substrate to bowl distance 912 and the bowl to chemical ceiling distance 908 is fixed (e.g., the addition of distances 912 and 908 is invariable) (e.g., in cases where the substrate to chemical ceiling distance 910 is a given distance). That is, in some cases, distances 912 and 908 are interdependent (e.g., as distance 912 increases by an amount, distance 908 will decrease by the same amount; and as distance 908 increases by an amount, distance 912 will decrease by the same amount).

In some cases, the substrate to bowl distance In some instances, the substrate to bowl distance 912 may be up to about 1.5 mm, about 1.6 mm, about 1.7 mm, about 1.8 mm, about 1.9 mm, about 2.0 mm, about 2.1 mm, about 2.2 mm, about 2.3 mm, about 2.4 mm, about 2.5 mm, about 2.6, about 2.7, about 2.8 mm, about 2.9 mm, about 3.0 mm, about 3.1 mm, about 3.2 mm, about 3.3 mm, about 3.4 mm, about 3.5 mm, about 3.6 mm, about 3.7 mm, about 3.8 mm, about 3.9 mm, about 4.0 mm, about 4.25 mm, about 4.5 mm, about 4.75 mm, about 5.0 mm, about 5.5 mm, about 6.0 mm, or about 6.5 mm. In some instances, the substrate to bowl distance 912 may be less than 1.5 mm. In some instances, the substrate to bowl distance 912 may be greater than 6.5 mm. In some instances, the substrate to bowl distances 912 may be within a range defined by any two of the preceding values. In some instances, the substrate to bowl distance 912 comprises a distance of at least 1.5 mm, at least 2.0 mm, at least 2.5 mm, at least 3.0 mm, at least 3.5 mm, at least 4.0 mm, at least 4.5 mm, at least 5.0 mm, at least 5.5 mm, at least 6.0 mm, or at least 6.5 mm.

In some cases, the bowl to chemical ceiling distance 908 may be up to about 0.1 mm, about 0.2 mm, about 0.3 mm, about 0.4 mm to about 1.2 mm. In some cases, the bowl to chemical ceiling distance 908 may be a distance of about 0.1 mm to about 0.2 mm, about 0.1 mm to about 0.3 mm, about 0.1 mm to about 0.4 mm, about 0.5 mm, about 0.6 mm, about 0.7 mm, about 0.8 mm, about 0.9 mm, about 1.0 mm, about 1.1 mm, about 1.2 mm, about 1.3 mm, about 1.4 mm, about 1.5 mm, about 1.6 mm, about 1.7 mm, about 1.8 mm, about 1.9 mm, about 2.0 mm, about 2.1 mm, about 2.2 mm, about 2.3 mm, about 2.4 mm, about 2.5 mm, about 2.6 mm, about 2.7 mm, about 2.8 mm, about 2.9 mm, about 3.0 mm, about 3.1 mm, about 3.2 mm, about 3.3 mm, about 3.4 mm, or about 3.5 mm. In some instances, the bowl to chemical ceiling distance 908 may be less than 0.1 mm. In some instances, the bowl to chemical ceiling distance 908 may be greater than 3.5 mm. In some instances, the bowl to chemical ceiling distance 908 may be within a range defined by any two of the preceding values. In some cases, the bowl to chemical ceiling distance 908 is a distance of at least 0.1 mm, at least 0.2 mm, at least 0.3 mm, at least 0.4 mm, at least 0.5 mm, at least 0.6 mm, at least 0.7 mm, at least 0.8 mm, at least 0.9 mm, at least 1.0 mm, at least 1.1 mm, at least 1.2 mm, at least 1.3 mm, at least 1.4 mm, at least 1.5 mm, at least 1.6 mm, at least 1.7 mm, at least 1.8 mm, at least 1.9 mm, at least 2.0 mm, at least 2.1 mm, at least 2.2 mm, at least 2.3 mm, at least 2.4 mm, at least 2.5 mm, at least 2.6 mm, at least 2.7 mm, at least 2.8 mm, at least 2.9 mm, or at least 3.0 mm.

In some instances, the total of the substrate to bowl distance 912 and the bowl to chemical ceiling distance 908 is a distance of at least 0.5 mm, at least 1 mm, at least 1.5 mm, at least 2 mm, at least 2.5 mm, at least 3 mm, at least 3.5 mm, at least 4 mm, at least 4.5 mm, at least 5 mm, at least 5.5 mm, at least 6 mm, at least 6.5 mm, at least 7 mm, at least 7.5 mm, at least 8 mm, at least 8.5 mm, at least 9 mm, at least 9.5 mm, or at least 10 mm. In some instances, the total of distances 908 and 912 may be within a range defined by any two of the preceding values.

In some cases, the distance 907 between the center 905 of the substrate and the fluid dispensers 904 (e.g., the center of the one or more fluid dispensers 904) may be up to about 0 mm, about 1 mm, about 2 mm, about 3 mm, about 4 mm, about 5 mm, about 6 mm, about 7 mm, about 8 mm, about 9 mm, about 10 mm, about 11 mm, about 12 mm, about 13 mm, about 14 mm, about 15 mm, about 16 mm, about 17 mm, about 18 mm, about 19 mm, or about 20 mm. In some cases, the distance 907 between the center 905 of the substrate and the fluid dispensers 904 may be greater than 20 mm. In some cases, the distance 907 between the center 905 of the substrate and the fluid dispensers 904 may be less than 0 mm. In some instances, the distance 907 between the center 905 of the substrate and the fluid dispensers 904 may be within a range defined by any two of the preceding values.

In some cases, the soak time (e.g., soak duration) may be up to about 1 s, about 2 s, about 3 s, about 4 s, about 5 s, about 6 s, about 7 s, about 8 s, about 9 s, about 10 s, about 15 s, about 20 s, about 25 s, about 30 s, about 35 s, about 40 s, about 50 s, or about 60 s. In some embodiments, the soak time may be longer than 60 s. In some embodiments, the soak time may be shorter than 1 s. In some instances, the soak time may be within a range defined by any two of the preceding values.

In some cases, the first velocity may be up to about 10 rotations per minute (rpm), about 20 rpm, about 30 rpm, about 40 rpm, about 50 rpm, about 60 rpm, about 70 rpm, about 80 rpm, about 90 rpm, or about 100 rpm. In some instances, the first velocity may be greater than 100 rpm. In some instances, the first velocity may be less than 10 rpm. In some instances, the first velocity may be within a range defined by any of the two preceding values.

In some cases, the second velocity may be up to about 320 rpm, about 340 rpm, about 360 rpm, about 380 rpm, about 400 rpm, about 420 rpm, about 440 rpm, about 460 rpm, about 480 rpm, about 500 rpm, about 520 rpm, about 540 rpm, about 560 rpm, about 580 rpm, about 600 rpm, about 620 rpm, about 640 rpm, about 660 rpm, about 680 rpm, about 700 rpm, or about 720 rpm. In some instances, the second velocity may be greater than 720 rpm. In some instances, the second velocity may be less than 320 rpm. In some instances, the second velocity may be within a range defined by any of the two preceding values.

In some cases, the first period of time (e.g., for removing the washing solution from the chemical ceiling and/or substrate) may be up to about 5 s, about 10 s, about 15 s, about 20 s, about 25 s, about 30 s, about 35 s, about 40 s, about 45 s, or about 50 s. In some instances, the first period of time may be longer than 50 s. In some instances, the first period of time may be shorter than 5 s. In some instances, the first period of time may be within a range defined by any two of the preceding values.

In some cases, the second period of time (e.g., for removing any remaining washing solution from the chemical ceiling and/or substrate) may be up to about 1 s, about 2 s, about 3 s, about 4 s, about 5 s, about 6 s, about 7 s, about 8 s, about 9 s, about 10 s, about 11 s, about 12 s, about 13 s, about 14 s, about 15 s, about 16 s, about 17 s, about 18 s, about 19 s, or about 20 s. In some instances, the second period of time may be greater than 20 s. In some instances, the second period of time may be shorter than 1 s. In some instances, the second period of time may be within a range defined by any two of the preceding values.

In some instances, the washing solution 1106 may be deposited at a volume up to about 140 mL, about 150 mL, about 160 mL, about 170 mL, about 180 mL, about 190 mL, about 200 mL, about 210 mL, about 220 mL, about 230 mL, about 160 mL, about 170 mL, about 180 mL, about 190 mL, about 200 mL, about 210 mL, about 220 mL, about 230 mL, about 240 mL, about 250 mL, about 260 mL, about 270 mL, about 280 mL, about 290 mL, or about 300 mL. In some instances, the volume of the washing solution 1106 that is deposited may be greater than 300 mL. In some instances, the volume of the washing solution 1106 that is deposited may be less than 140 mL. In some instances, the volume of the washing solution 1106 that is deposited may be within a range defined by any two of the preceding values. In some instances, the volume of washing solution 1106 that is used depends at least in part on the total of the substrate to bowl distance 912 and the bowl to chemical ceiling distance 908 (e.g., as the total distance between the substrate and chemical ceiling increases, the volume of washing solution that is used may increase). In some instances, the volume of washing solution 1106 that is used depends at least in part on the distance 907 between the center 905 of the substrate and the fluid dispensers 904 (e.g., as the distance between the center of the substrate and the fluid dispensers increases, the volume of washing solution that is used may increase). In some instances, the volume of washing solution 1106 that is used depends at least in part on the total of the substrate to bowl distance 912 and the bowl to chemical ceiling distance 908 and depends at least in part on the distance 907 between the center 905 of the substrate and the fluid dispensers 904.

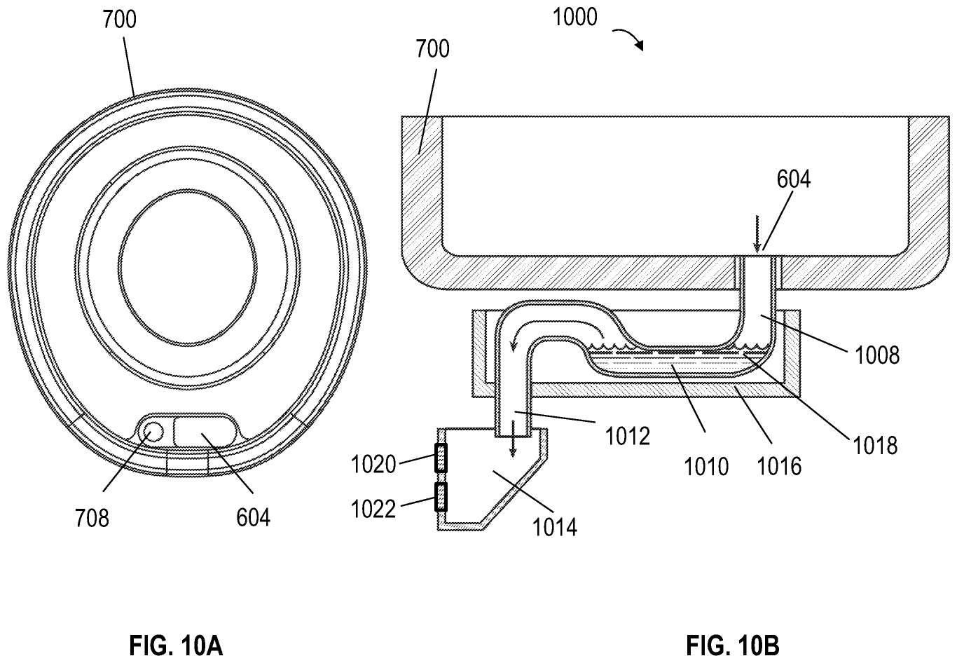

In some instances, provided herein is a bowl that is in fluid communication with a drain assembly 1000, as seen in FIGS. 10A-10B. FIG. 10A depicts a top view of the bowl 700. In some cases, the bowl 700 may be in fluid communication with a fluidic drain assembly (e.g., comprising elements 1008, 1010, 1012). In some embodiments, an upper bracket (not known) and lower bracket (1016) are configured to support the fluidic drain assembly that is in fluid communication with the bowl 700. In some cases, the drain assembly may comprise a lower bracket 1016 in mechanical communication with an upper bracket (not shown) in mechanical communication with the bowl 700. In some cases, the upper bracket and lower bracket 1016 may be mechanically fastened to one another by one or more fasteners. In some cases, the fasteners may comprise a machine screw and/or bolt. In some instances, the lower bracket 1016 may be configured to mechanically support the fluidic.

The fluidic drain assembly (e.g., comprising elements 1008, 1010, 1012) may be in fluid communication with the bowl via a drain 604 on the bottom surface of the bowl. In some instances, the drain may comprise a filter disposed over the drain 604 configured to prevent large particles or residue to drain into the fluidic drain assembly and clog the fluidic system. In some cases, the bowl may comprise a fluid sensor 708 (e.g., one or more fluid or temperature sensors) configured to sense a fluid level collecting at the bottom of the bowl 700 (e.g., residual fluid 916). The fluid sensor may be in electrical communication with one or more processors of a system, described elsewhere herein, configured to provide (e.g., to the one or more processors) an electrical signal indicative of the level of the fluid draining from the bowl. In some instances, the fluid sensor may provide (e.g., to the one or more processors) a voltage signal indicating that drain is clogged indicating a risk of the fluid overflowing over the edge of the bowl. In some instances, a voltage signal (e.g., electrical communication) from the fluid sensor(s) may be transmitted to the one or more processors. In some cases, fluid may drain from the bowl 700 passively (i.e., through the force of gravity acting on the fluid) through the drain assembly.

In some cases, the drain assembly may comprise a pair of right angle fluidic couplers (1012, 1018), where the first right angle fluidic coupler 1018 is in fluidic communication with the drain of the bowl, an interconnecting segment of tubing 1010, and a second right angle fluidic coupler 1012. In some cases, the interconnecting segment of tubing 1010 may comprise a curvature configured to maintain passive draining of the fluid from the bowl to the second right angle fluidic coupler 1012.

In some instances, the bowl in fluid communication with a drain assembly 1000 may be in further fluid communication with a trough 1014, as seen in FIG. 10B. In some cases, one or more bowls, in fluid communication with drain assemblies 1000, may simultaneously be in fluidic communication with the trough 1014. In some cases, the trough may comprise a fluid sensor (not shown) configured to detect a level of fluid in the trough. In some instances, the fluid sensor may be in electrical communication with a processor of a system, described elsewhere herein, to indicate a level of fluid in the trough (e.g., fluid at risk of overflowing the trough). In some instances, the trough 1014 may comprise a passive fluid drain 1020 and/or an active drain 1022 (e.g., located lower than the passive fluid drain on the same side or a different side of the trough) in fluid communication with the trough 1014 and/or a downstream fluidic system. In some cases, the active drain may be in fluid communication with a pump that is configured to provide a negative and/or vacuum pressure in fluid communication with active drain 1022. In some instances, the active drain may continually provide the negative and/or vacuum pressure in fluid communication with the active drain 1022 regardless of the presence or lack thereof fluid in the trough. In some cases, the passive drain 1020 may be configured to drain liquid from the trough 1014 when the fluid level in the trough reaches the head of the passive drain 1020. As depicted in FIG. 10B, tubing 1010 is at a lower position than the portion of the tubing (e.g., element 1002) leading to the outlet connected to the trough. As a result, fluid 1018 accumulates in tubing 1010 and will only drain when the fluid level reach at a certain level. The accumulated fluid creates a seal that separate the reaction environment inside bowl 700 from outside environment, ensuring that the reaction environment maintains uniform or near uniform reaction conditions including humidity, temperature, etc.

In some instances, the pump may be in electrical communication with a processor of the system, described elsewhere herein, configured to control whether or not the pump is enabled and/or the pump pressure applied at the active drain 1020. In some cases, the pump may be in fluidic communication with an inlet tubing, outlet tubing, one or more right angle tubing couplers, straight tubing couplers, or any combination thereof.

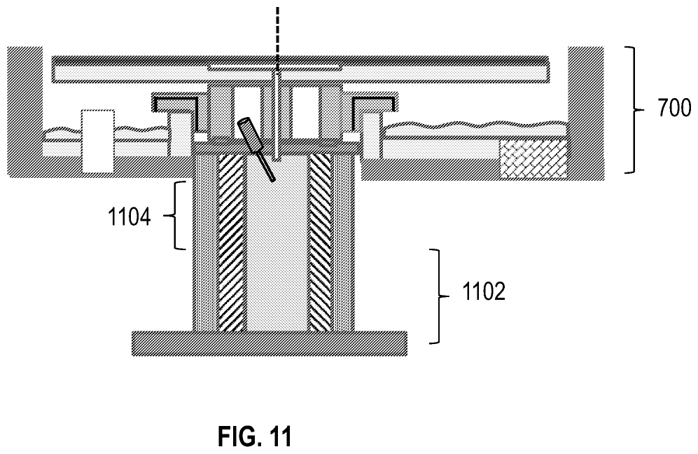

In some cases, the substrate temperature may be controlled by a heater and/or cooler (e.g., 1102) thermally coupled to a thermistor (e.g., 1104), rotor 814 of the motor, chuck 707, substrate 706, or any combination thereof, as seen in FIGS. 7 and 11. In some instances, the substrate 706 may be maintained at a constant temperature by increasing or decreasing the temperature of the heater and/or cooler 1102. In some cases, motor 712 generates heat as it rotates, and this heat can be used for maintaining substrate 806 at a constant temperature. In some cases, thermal properties of the material thermistor 1104, rotor 814 of the motor, chuck 707, substrate 706, or any combination thereof may provide thermal insulating or thermal sink properties to maintain a constant temperature of the substrate 706. In some instances, the chemical ceiling 900 may be cooled and/or heated to maintain a constant temperature of the substrate 706. In some cases, maintaining a constant temperature of the substrate may provide more consistent results than those achieved from a substrate that fluctuates in temperature.

In some instances, where the sequencing system is operated with heater and/or cooler 1102, the temperature of the substrate (e.g., the wafer) remains constant as the motor rotates. In contrast, in some instances where the sequencing system does not utilize the heater and/or cooler 1102, fluctuations in the temperature of one or both of the substrate (e.g., the wafer), and chuck may be observed.

### Real-Time Operations and Instructions

The sequencing system of the present disclosure permits highly efficient sequencing operation. Such efficiency may be facilitated by allowing parallel real-time operations and/or instructions, such as dynamic queuing and hot-swapping of samples for processing, real-time replacement and/or replenishing of reagents, and real-time loading and/or unloading of substrates. The term “real-time,” as used herein, generally refers to simultaneous or substantially simultaneous occurrence of, or without interruption, of one event (e.g., updating sample queuing instructions) relative to occurrence of another event (e.g., processing of another sample).

In some examples, the methods and systems provided herein may facilitate high-throughput, continuous, automated, and/or un-interrupted sequencing of one or more samples. The samples that may be used along with the sequencing system of the present disclosure are described in further detail elsewhere herein. In some examples, the sample may comprise a plurality of particles (e.g., beads). A particle (e.g., bead) of the plurality of particles (e.g., beads) may comprise one or more (e.g., a plurality of) nucleic acid molecules (e.g., DNA and/or RNA molecules) coupled thereto (e.g., immobilized thereon). The nucleic acid molecules may have been immobilized on the surface of the particles prior to sample loading on the sequencing system. Nucleic acid molecules of a given sample may derive from a same source, such as a same subject. Alternatively, nucleic acid molecules of a given sample may derive from one or more different sources, such as one or more different subjects. In some examples, the methods and systems provided herein may facilitate high-throughput, continuous, automated, and/or un-interrupted sequencing of a plurality of samples deriving from a plurality of sources.

Provided herein is a system (e.g., 100 of FIG. 1) for sequencing a plurality of nucleic acid samples, comprising: (i) a processing station (e.g., 104) configured to bring a nucleic acid molecule of a nucleic acid sample immobilized adjacent to a substrate (e.g., coupled to a substrate via a particle, as described herein) into contact with a reagent to sequence the nucleic acid molecule; (ii) a sample station (e.g., 101) configured to provide the nucleic acid sample to the processing station; (iii) a substrate station (e.g., 102) configured to provide the substrate to the processing station, which substrate is configured for immobilization of the nucleic acid molecule adjacent thereto; (iv) a reagent station (e.g., 103) configured to provide the reagent to the processing station, wherein the reagent is obtained from a first reservoir and/or a second reservoir; and (v) a controlling station (e.g., 107) comprising one or more processors that are individually or collectively programmed to execute (1) at least a portion of a first queuing instruction to introduce a first set of one or more nucleic acid samples of the plurality of nucleic acid samples, including the nucleic acid sample, from the sample station to the sequencing station according to a first order of introduction defined by the first queuing instruction; (2) a substrate loading instruction to introduce the substrate from the substrate station to the sequencing station and immobilize the first set of one or more nucleic acid samples adjacent to the substrate; and (3) a sequencing instruction to draw the reagent alternately from the first reservoir or the second reservoir, or alternately from the first reservoir and the second reservoir, and deliver the reagent to the sequencing station. The processing station may be capable of operating during performance of any one or more other actions, such as (1) introducing an additional nucleic acid sample to the sample station; (2) inputting a second queuing instruction and executing at least a portion of said second queuing instruction, wherein the second queuing instruction defines a second order of introduction that is different than the first order of introduction; (3) introducing an additional substrate to the substrate station; and/or (4) introducing an additional volume of the reagent to the reagent station by one or more (i) replacing the first reservoir or the second reservoir with a third reservoir containing the reagent and (ii) replenishing the first reservoir or the second reservoir with the reagent. In some instances, the processing station is capable of operating for at least 24 hours without human intervention (e.g., as described herein).

Provided herein is a method for sequencing a plurality of nucleic acid samples. The method can comprise providing a nucleic acid sequencer having (i) a processing station configured to bring a nucleic acid molecule of a nucleic acid sample immobilized adjacent to a substrate (e.g., coupled to a substrate via a particle, as described herein) into contact with a reagent to sequence the nucleic acid molecule; (ii) a sample station configured to provide the nucleic acid sample to the sequencing station; (iii) a substrate station configured to provide the substrate to the sequencing station, which substrate immobilizes adjacent thereto the nucleic acid sample; and (iv) a reagent station configured to provide the reagent to the sequencing station, wherein the reagent is obtained from a first reservoir and/or a second reservoir. The method can comprise executing, by one or more processors individually or collectively, (i) at least a portion of a first queuing instruction to introduce a first set of one or more nucleic acid samples of the plurality of nucleic acid samples, including the nucleic acid sample, from the sample station to the sequencing station according to a first order of introduction defined by said first queuing instruction; (ii) a substrate loading instruction to introduce the substrate from the substrate station to the sequencing station and immobilize said first set of one or more nucleic acid samples adjacent to the substrate; and (iii) a sequencing instruction to draw the reagent from the first reservoir or the second reservoir, or alternately from the first reservoir and the second reservoir, and deliver the reagent to the sequencing station. The method may comprise, while the processing station is in operation, performing any one or more other actions, such as (1) introducing an additional nucleic acid sample to the sample station; (2) inputting a second queuing instruction and executing at least a portion of said second queuing instruction, wherein the second queuing instruction defines a second order of introduction that is different than the first order of introduction; (3) introducing an additional substrate to the substrate station; and (4) introducing an additional volume of the reagent to the reagent station by one or more (i) replacing the first reservoir or the second reservoir with a third reservoir containing the reagent and (ii) replenishing the first reservoir or the second reservoir with the reagent. In some instances, the processing station is capable of operating for at least 24 hours without human intervention (e.g., as described herein).

The method of the present disclosure may comprise hot swapping. Hot swapping may comprise hot swapping (e.g., substituting in real-time, while one or more processes are in progress, and/or while power is connected to the system) reagents, substrates (e.g., wafers), and/or samples. In some cases, each of the reagents, substrates and samples may be hot-swapped. Similarly, additional reagents, substrates, and/or samples may be added during operation of the sequencing system. Similarly, existing reagents, substrates, and/or samples not in use may be removed during operation of the sequencing system. Provided herein are also methods for sample loading. Sample loading may comprise a variety of techniques described in further detail elsewhere herein. In some cases, in order to facilitate a high throughput and/or un-interrupted workflow for sequencing which may be automated and at least partially independent of the operator, hot swapping of a number of samples may be performed. In some examples, the samples which may be more important, expensive, or precious than other samples may not be loaded in the beginning of a process, such as a sequencing process.

In some examples, a user or an automated or robotic system may change the order of samples to be sequenced (e.g., at any time). Some samples may be designated as low or lower priority samples (e.g., via user input at a user interface) and may be loaded later than other samples, loaded in areas of a substrate that will not be interrogated and/or will be later interrogated, and/or loaded onto a substrate that will be processed after another, higher priority substrate. Similarly, certain samples may be designated as high or higher priority samples (e.g., via user input at a user interface) and may be loaded prior to other samples, loaded in areas of a substrate that will interrogated earlier in a processing and/or detection process, and/or loaded onto a substrate that will be processed prior to another, lower priority substrate. Lower priority samples may be placed lower in a sample processing queue, while higher priority samples may be placed higher in a sample processing queue. To facilitate this, more than one sample port via which a sample may be loaded onto a substrate may be provided and used in the system. For example, the system may comprise 1, 2, 3, 4, 5, 6, 7, 8, 9, 10, 12, 15, 20, 25, 30, 35, 40, or more sample ports. The system may comprise several sample ports. Multiple (e.g., several) sample ports may facilitate a dynamic queue. In some cases, the order of the samples to be loaded in the queue may not change frequently or dynamically. In some cases, multiple samples may be processed on a single substrate (e.g., wafer). The system may comprise an algorithm for loading one or more samples on a substrate and performing efficient sequencing and/or other processes. In some cases, the sequencer may be operated in different sequencing modes, for example, optimized for different conditions such as different flow orders or other conditions. In some examples, samples requiring a similar mode can be run together. In an example, one or more samples from an existing sample queue may be replaced with another sample not currently included in a sample queue. A sample queue may comprise a physical organization of samples within a system. Alternatively or additionally, a sample queue may comprise an indexed organization of samples within one or more processors of the system. A system may comprise a high priority sample storage area and/or one or more high priority sample ports. A system may be configured to load samples designated as high priority and optionally stored in a high priority sample storage area or moved to a high priority position in a physical sample queue directly onto a substrate (e.g., wafer) to expedite analysis of the high priority samples.

In some instances, a processing station and/or a detection station may be disposed in a different environment than that/those of one or more other stations, such as the sample station, substrate station, and/or reagent station. The environment of the processing station and/or the detection station may have a higher relative humidity than the other environments. In some instances, the environment of the processing station and/or the detection station may comprise one or more local regions of controlled local environments (e.g., local temperature, local humidity) that are different from the other environments (e.g., as described herein).

In some instances, a processing station and/or a detection station may be disposed in a different environment than an ambient environment. The environment of the processing station and/or the detection station may have a higher relative humidity than the ambient environment. In some instances, the environment of the processing station and/or the detection station may comprise one or more local regions of controlled local environments (e.g., local temperature, local humidity) that are different from the ambient environment.

In some instances, one or more stations, such as the sample station, substrate station, and/or the reagent station, may comprise a sealed environment. For example, the substrate station can comprise a hermetically sealed environment. In some instances, the substrate station can comprise a vacuum desiccator. In some cases, the system may comprise a mechanism for removing impurities and/or contaminants. For example, a reagent handling and/or diluent handling system, or another component of a system, may comprise one or more filters for removing one or more impurities and/or contaminants. Such a filter may be configured to remove agglomerated materials (e.g., a size-based filter) and/or charged materials. For example, a reagent handling and/or diluent handling system, or another component of a system, may comprise a carbon filter, a reverse osmosis system, an ionizer, a UV filter, an IR filter, a ceramic filter, an activated alumina filter, or any other useful system. The system may further comprise a mechanism for replacing depleted active components. For example, the system (e.g., a reagent handling and/or diluent handling system) may comprise a mechanism for reconstituting a material comprising a reagent or diluent, such as by addition of water or another material (e.g., following evaporation or other depletion of a portion of the material).

In some instances, a method may further comprise purifying a reagent mixture comprising a reagent prior to delivery of the reagent to the processing station, wherein the reagent mixture comprises a plurality of nucleotides or nucleotide analogs (e.g., as described herein). In some instances, purification may comprise (A) directing the reagent mixture to a reaction space comprising a support having a first plurality of nucleic acid molecules immobilized adjacent thereto; (B) incorporating a subset of nucleotides or nucleotide analogs from the plurality of nucleotides or nucleotide analogs into the first plurality of nucleic acid molecules, thereby providing a remainder of the plurality of nucleotides or nucleotides analogs, wherein (B) is performed without detecting the subset of nucleotides incorporated into the plurality of nucleic acid molecules; and (C) delivering the remainder of the plurality of nucleotides or nucleotide analogs to the processing station. In some instances, the method can further comprise (D) incorporating at least a subset of the remainder of the plurality of nucleotides or nucleotides analogs into a growing stand associated with the nucleic acid molecule.

In some instances, the subset of nucleotides or nucleotide analogs comprises less than 10%, less than 5%, less than 1%, less than 0.1%, or less than 0.01% of the plurality of nucleotides or nucleotide analogs. In some instances, the remainder of the plurality of nucleotides or nucleotide analogs has a ratio of a number of nucleotides or nucleotide analogs of one or more but less than all canonical types to a number of nucleotides or nucleotide analogs of all other canonical types which is greater than 19:1. In some instances, the ratio is at least about 29:1, 99:1, or 999:1.

In some instances, purification may comprise (A) selecting from a set of canonical types of nucleotides or nucleotides analogs a subset of canonical types of nucleotides or nucleotide analogs; (B) directing the reagent mixture to a reaction space comprising a support having a plurality of nucleic acid molecules immobilized thereto, wherein a percentage of nucleotides or nucleotide analogs corresponding to the subset relative to all other nucleotides or nucleotide analogs in the mixture is greater than 50%; and (C) incorporating nucleotides or nucleotide analogs from the mixture that do not correspond to the subset into the plurality of nucleic acid molecules such that the percentage is increased following the incorporating, wherein (A)-(C) are performed in absence of sequencing or sequence identification of the plurality of nucleic acid molecules. In some instances, purification may comprise (A) directing the reagent mixture to a reaction space comprising a support having a plurality of nucleic acid molecules immobilized thereto; and (B) incorporating a subset of nucleotides or nucleotide analogs form the plurality of nucleotides or nucleotide analogs into the plurality of nucleic acid molecules, thereby providing a remainder of the plurality of nucleotides or nucleotides analogs, wherein (A)-(B) are performed in absence of sequencing or sequence identification of the plurality of nucleic acid molecules. In some instances, the method can further comprise (C) using the remainder of the plurality of nucleotides or nucleotide analogs to perform nucleic acid sequencing by synthesis.

Provided herein is a method for processing analytes, comprising executing, by one or more processors individually or collectively, at least a portion of a first queuing instruction to introduce a first set of one or more sample analytes from a sample station into a processing station according to a first order of introduction defined by the first queuing instruction, wherein the sample station comprises a plurality of sample sources, wherein each of the plurality of sample sources is accessible for introduction of sample analytes from the plurality of samples sources into the processing station by one or more controllers, and wherein the first queuing instruction defines the first order of introduction of the sample analytes between the plurality of sample sources. The method may further comprise receiving a second queuing instruction, wherein the second queuing instruction defines a second order of introduction different from the first order of introduction. The method may further comprise executing, by the one or more processors individually or collectively, at least a portion of the second queuing instruction to introduce a second set of one or more sample analytes from the sample station to the processing station according to the second order of introduction while the processing station is in operation.

Where a sample station comprises a plurality of samples (e.g., a plurality of sample sources), a sample may be introduced from the sample station to the processing station, such as onto a substrate in the processing station, according to a defined order of introduction. A current queuing instruction may comprise the order of introduction and/or a set of rules for determining the order of introduction. The queuing instruction may be or comprise a default set of instructions, such as to be followed absent user instructions. For example, the default set of instructions may define an order of introduction according to an order that the sample sources were loaded into the sample station (e.g., first loaded to sample station is the first loaded to processing station), or the reverse. In another example, the default set of instructions may define an order of introduction according to a location of the sample source in the sample station (e.g., a sample source in a first coordinate is loaded first, a sample source in a second coordinate is loaded next, etc.). Alternatively or additionally, the queuing instruction may be or comprise user instructions. The user instructions may define a specific order of introduction of the plurality of samples to the processing station, and/or a set of rules to follow for order of introduction of the plurality of samples to the processing station.

In some instances, the sequencing system may operate under a first queuing instruction (e.g., default and/or user-provided) such that the processing station is in operation. During such operation, the system can receive a second queuing instruction that defines a different order of introduction of the plurality of samples to the processing station than the first queuing instruction. One or more processors may execute at least a portion of the second queuing instruction while the processing station is in operation, such as until an updated queuing instruction (e.g., third queuing instruction) is received. The second queuing instruction may be executed without terminating the operation of the processing station.

For example, conditions of operation of the processing station may not be disturbed during such queuing instruction update. Such conditions may include maintaining a sample environment (e.g., modular sample environment) in the processing station at a different environment than an ambient environment and/or uncontrolled environment. The processing station may be maintained at a different environment than an environment of the sample station.

In some instances, the processing station can be maintained at a different temperature than an ambient temperature. In some instances, the sample environment (or any element thereof) in the processing station may be maintained at a temperature of at least about 20 degrees Celsius (° C.), 25° C., 30° C., 35° C., 40° C., 45° C., 50° C., 55° C., 60° C., 65° C., 70° C., 75° C., 80° C., 85° C., 90° C., 95° C., 100° C., or higher. Alternatively, the sample environment may be maintained at less than 20° C. Alternatively or additionally, the sample environment (or any element thereof) in the processing station may be maintained at a temperature of at most about 100° C., 95° C., 90° C., 85° C., 80° C., 75° C., 70° C., 65° C., 60° C., 55° C., 50° C., 45° C., 40° C., 35° C., 30° C., at 25° C., 20° C., or lower. The sample environment may be maintained at a temperature that is within a range defined by any two of the preceding values. Different elements of the sample environment, such as the chamber, protruding portion of the detector, one or more optical elements, immersion fluid, plate, substrates, solutions, and/or samples therein may be maintained at different temperatures or within different temperature ranges, such as the temperatures or temperature ranges described herein. Elements of the system may be set at temperatures above the dewpoint to prevent condensation. Elements of the system may be set at temperatures below the dewpoint to collect condensation.

In some instances, the processing station can be maintained at a different humidity than an ambient humidity. In some instances, the sample environment in the processing station may be maintained at a relative humidity of at least about 5%, 10%, 15%, 20%, 25%, 30%, 35%, 40%, 45%, 50%, 55%, 60%, 65%, 70%, 75%, 80%, 85%, 90%, 95%, or higher, such as about 100%. Alternatively or additionally, the relative humidity may be maintained at a level of at most about 100%, 95%, 90%, 85%, 80%, 75%, 70%, 65%, 60%, 55%, 50%, 45%, 40%, 35%, 30%, 25 20%, 15%, 10%, 5%, or less. Alternatively or additionally, the relative humidity may be maintained within a range defined by any two of the preceding values.

An environmental unit (e.g., humidifiers, heaters, heat exchangers, compressors, etc.) may be configured to regulate one or more operating conditions in each sample environment (e.g., modular sample environment). In some instances, each environment may be regulated by independent environmental units. In some instances, a single environmental unit may regulate a plurality of environments. In some instances, a plurality of environmental units may, individually or collectively, regulate the different environments. An environmental unit may use active methods or passive methods to regulate the operating conditions. For example, the temperature may be controlled using heating or cooling elements. The humidity may be controlled using humidifiers or dehumidifiers.

In some instances, a first part of the sample environment may be further controlled from other parts of the sample environment. Different local parts may have different local temperatures, pressures, and/or humidity, which local temperatures, pressures, and/or humidity may be separately controlled and/or controlled in a concerted manner (e.g., as described herein). For example, the sample environment may comprise a first internal or local environment and a second internal or local environment, for example separated by a seal. In some instances, the seal may comprise an immersion objective lens, as described elsewhere herein. For example, the immersion objective lens may be part of a seal that separates the sample environment into a first internal environment having 100% (or substantially 100%) relative humidity and a second environment having a different temperature, pressure or humidity.

In some instances, a queuing instruction may comprise a sample selection instruction. In some instances, a substrate may be capable of receiving and/or processing a plurality of samples (e.g., from a plurality of sample sources). In some instances, a group of samples to be loaded onto a substrate can be selected according to the sample selection instruction. For example, the sample selection instruction may be based at least in part on use of the substrate area, such that a group of selected samples for a substrate use the substrate area most effectively. In some instances, the sample selection instruction may be based at least in part on processing conditions or protocols (e.g., common processing conditions or common protocols, e.g., sequencing protocol) that can be used to process a group of selected samples. For example, the group of selected samples may be selected for deposition on a substrate, wherein the group of selected samples may each be processed using a first set of conditions which differs from a second set of conditions at which other samples are processed.

In some instances, while the processing station is in operation, the contents of the sample station may be updated such as to add a new sample source to, remove an existing sample source from, or change locations of different sample sources within, the sample station. For example, after loading the new sample source into the sample station during operation of the processing station (e.g., after executing at least a portion of the first queuing instruction), a second queuing instruction may comprise an order of introduction that directs for the new sample source to be delivered to the processing station prior any pre-existing sample sources in the sample sources. Beneficially, such dynamic sample introduction and queuing for processing in the sequencing system may allow accommodation of real-time priority updates.

Provided herein is a method for processing analytes, comprising providing a first reagent source (e.g., reservoir) and a second reagent source (e.g., reservoir) in a reagent station, wherein each of the first reagent source and the second reagent source (i) comprises a first reagent, and (ii) is accessible for introduction of the first reagent from the reagent station to a processing station by a controller, wherein the processing station is configured to facilitate one or more operations using the first reagent. The method may further comprise directing the first reagent from the first reagent source to the processing station. The method may further comprise directing the first reagent from the second reagent source to the processing station. The method may further comprise, while the processing station is in operation and receiving the first reagent from the second reagent source, (i) replacing the first reagent source with a third reagent source comprising the first reagent, wherein the third reagent source is accessible for introduction of the first reagent from the reagent station to the processing station by the controller, or (ii) replenishing the first reagent source with an additional volume of the first reagent. The method may further comprise, directing the first reagent from (i) the third reagent source, or (ii) the additional volume of the first reagent in the first reagent source, to the processing station. The controller may be configured to control one or more actuators, and/or one or more valves in fluid communication with the first reagent source or the second reagent source, to direct the first reagent from the first reagent source or the second reagent source to the processing station.

In some instances, the first reagent may be drawn from the second reagent source when the first reagent source has depleted below a predetermined threshold level. In some instances, the predetermined threshold level may be a fully depleted level. Alternatively, the predetermined threshold level may be any depletion level. In some instances, the first reagent may be drawn from the third reagent source or from the additional volume of the replenished first reagent source when the second reagent source has depleted below a predetermined threshold level. In some instances, the predetermined threshold level may be a fully depleted level. Alternatively, the predetermined threshold level may be any depletion level. In some instances, the two predetermined threshold levels may be the same or different.

In some instances, the method may further comprise diluting the first reagent with a diluent subsequent to departure of the first reagent from the reagent station and prior to delivery to the processing station. The diluent may be or comprise water, such as deionized water. The diluent may be drawn from a diluent reservoir, such as from the diluent station as described elsewhere herein. In some instances, the diluent may be produced and/or generated at the diluent station within an enclosure of the sequencing system as described elsewhere herein.

In some instances, the replacing and/or replenishing with additional volumes of the reagent may be accomplished without terminating operation(s) of the processing station. For example, conditions of operation of the processing station may not be disturbed during such reagent source switching. Such conditions may include maintaining a sample environment (e.g., modular sample environment) in the processing station at a different environment than an ambient environment and/or uncontrolled environment, such as at different temperatures and/or humidifies described elsewhere herein. The processing station may be maintained at a different environment than an environment of the reagent station, such as at different temperatures and/or humidifies.

In some instances, such as for sequencing applications, the processing station can be configured to direct the reagent to contact an analyte in the processing station. The process station and/or the detection station can be configured to detect a signal or signal change associated with the analyte. In some instances, the analyte can be a nucleic acid molecule and the first reagent may comprise one or more of a solution comprising a plurality of nucleotides (e.g., a solution comprising adenine-containing nucleotides, a solution comprising cytosine-containing nucleotides, a solution comprising thymine-containing nucleotides, a solution comprising uracil-containing nucleotides, or a solution comprising guanine-containing nucleotides), an enzyme or enzyme-containing solution, a wash buffer, a cleavage solution (e.g., to cleave a fluorescent label from a nucleotide), and the like. The reagent station may comprise a plurality of types of reagent (e.g., such as each of the ones listed above), each comprising multiple reagent sources such that each type of reagent is replaceable and/or replenishable.

Provided is a method for processing analytes, comprising providing a plurality of substrates in a substrate station, wherein each of the plurality of substrates is accessible for introduction of substrates from the substrate station into a processing station by one or more actuators. The method can comprise delivering, by one or more actuators, a first substrate of the plurality of substrates into the processing station. The method can further comprise, in the processing station, performing an operation involving an analyte immobilized adjacent to the first substrate. The method can further comprise delivering, by the one or more actuators, a second substrate of the plurality of substrates into the processing station while the processing station is performing the operation.

In some instances, the substrate station can comprise an array (e.g., rack) containing the plurality of substrates. In some instances, the array (e.g., rack) can be a vertical rack that is configured to contain the plurality of substrates in a substantially horizontal position. In some instances, the array (e.g., rack) can be a horizontal rack that contains the plurality of substrates in a substantially vertical position.

In some instances, the first substrate and/or the second substrate may be any of the substrates described elsewhere herein. For example, a substrate may be planar or substantially planar. The substrate may be an open substrate. The substrate may not be, or part of, a flow cell (e.g., such as distinguished from flow cell cartridges). The substrate may be patterned or textured.

In some instances, the delivery of substrates may be accomplished without terminating operation(s) of the processing station. For example, conditions of operation of the processing station may not be disturbed during such reagent source switching. Such conditions may include maintaining a sample environment (e.g., modular sample environment) in the processing station at a different environment than an ambient environment and/or uncontrolled environment, such as at different temperatures and/or humidifies described elsewhere herein. The processing station may be maintained at a different environment than an environment of the substrate station, such as at different temperatures and/or humidifies.

In some instances, the processing station can be configured to process operations on two or more substrates simultaneously. In some instances, two or more processing stations can be configured to process operations on two or more substrates simultaneously. In some instances, a processing station and a detection station may be configured to process operations on two or more substrates simultaneously.

In some instances, such as for sequencing applications, the processing station can be configured to deposit an analyte from a sample onto the first substrate. The processing station can be configured to direct a reagent to contact the analyte immobilized adjacent to the first substrate. In some instances, the processing station and/or the detection station can be configured to detect a signal or signal change associated with the analyte.

Provided herein is a method for processing analytes, comprising inputting (1) a plurality of nucleic acid samples from different sample sources, and (2) a plurality of substrates, and providing, to one or more processors, user instructions to start two or more sequencing cycles. The method may comprise, in a first sequencing cycle, processing a first nucleic acid sample from the plurality of nucleic acid samples on a first substrate of the plurality of substrates, and during or subsequent to the first sequencing cycle, in a second sequencing cycle, processing a second nucleic acid sample from the plurality of nucleic acid samples on a second substrate of the plurality of substrates, wherein the second sequencing cycle is performed in absence of additional user intervention.

In some instances, the method may comprise, during or subsequent to an (n−1)^{th} sequencing cycle, in an nth sequencing cycle, processing an nth nucleic acid sample from the plurality of nucleic acid samples on an nth substrate of the plurality of substrates, wherein the nth sequencing cycle is performed in absence of additional user instructions from the user instructions.

Using the systems, methods, and devices provided herein, the sequencing system may be able to run, without user intervention (e.g., subsequent to an initiation), for at least about 1, 2, 3, 4, 5, 6, 7, 8, 9, 10, 11, 12, 13, 14, 15, 16, 17, 18, 19, 20, 21, 22, 23, 24, 36, 48, 60, 72 hours or more.

Provided herein is a system for processing an analyte comprising one or more stations described herein, and one or more processors, individually or collectively programmed to, within at most 40 hours of running time of the processing station, output at least about 1.5 giga reads per substrate. Alternatively or additionally, the output may be at least about 1.0, 2.0, 3.0, 4.0, 5.0, 6.0, 7.0, 8.0, 9.0, 10.0, 20.0, 30.0, 40.0, or 50.0 giga reads per substrate or more.

Alternatively or additionally, the one or more processors can, individually or collectively programmed to, within at most 40 hours of running time of the processing station, output sequence reads averaging at least 140 base pairs (bp) in length. Alternatively or additionally, the output may be at least about 100, 110, 120, 130, 140, 150, 200, 250, 300, 350, 400, 450, 500 bp read length or more.

Alternatively or additionally, the one or more processors can, individually or collectively programmed to, within at most 40 hours of running time of the processing station, output at least 0.2 terabase reads per run. Alternatively or additionally, the output may be at least about 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0 terabase reads per run or more.

Beneficially, the systems and methods of the present disclosure may facilitate automated sequencing with minimum user intervention, or in some cases, with lack of user intervention, after initiation of the automated process. The methods and systems of the present disclosure may increase automation efficiency by implementing one or more sensors with a control system. Control systems may be implemented as computer systems, such as comprising one or more processors or microprocessors, which are individually or collectively configured to perform certain operations, which are described elsewhere herein, such as with respect to FIG. 6. A control system may be in operable communication with mechanical controllers (e.g., actuator components, environmental units, movement units, etc.) as well as a sensor, or a combination of sensors, which provide measurements on a state or change in a component or process of the automated sequencing system. In non-limiting examples, the sensors may include temperature sensors, pressure sensors, humidity or moisture sensors, weight sensors (e.g., load cells), friction sensors, flow meters, motion sensors, optical sensors (e.g., cameras), pH sensors, audio sensors, voltage, current, and/or resistive sensors. The sensor may be any device or system capable of detecting a signal on a state or change in a component or process of the automated sequencing system. The sensor may automatically, and/or upon request, detect and transmit a signal to the control system, which may analyze the signal to determine a conclusion and based on the conclusion instruct one or more mechanical controllers to adjust, calibrate, or maintain a component or process of the automated sequencing system. The feedback may be open loop control feedback and/or a closed loop control feedback. In some instances, predetermined values (or ranges) or predetermined threshold values, as measured by one or more sensors, may be associated for a given component or process of the automated sequencing system, and the control system may be configured to instruct the appropriate mechanical controllers to adjust, calibrate, and/or maintain the given component or process when a predetermined threshold value is crossed. In some instances, the control system may be configured to instruct the appropriate mechanical controllers to adjust, calibrate, and/or maintain the given component or process at or near the predetermined value (or range). The control system may be in operable communication with a network of sensors to control one or more components or processes of the automated sequencing system, such as components or processes of the various stations described elsewhere herein.

In an example, to facilitate timely and precise hot-swapping of reagents, as described elsewhere herein, one or more sensors may be provided in the automated sequencing system to detect that a reagent reservoir needs replenishing or replacing. For example, a load cell may be used to determine a volume or mass of the reagent remaining in the reservoir from a weight of the reservoir, or a camera may be used to determine a volume level of the reservoir. The sensor(s) may continuously monitor the reagent reservoirs, or collect and transmit values upon request. In some cases, a predetermined value can be set as a predetermined threshold for alerting the control system. Upon receipt of an alert and/or determining that the reservoir needs replenishing or replacing (or otherwise that reagent may no longer be drawn from the reservoir), the control system may instruct that the drawing machine draw from the next available reservoir such that the sequencing process is not interrupted. The control system may also inform the operator that the first reservoir needs replenishing or replacing by sending an alert. Alternatively or in addition, the first reservoir may be automatically replenished or replaced. In another example, to facilitate optimal sample processing conditions in a sample environment system (e.g., 305a, 305b in FIGS. 3A-3C), one or more temperatures sensors and/or humidity sensors may be configured to detect the temperature and humidity of a sample environment to ensure that optimal temperature and humidity ranges are maintained during chemical processing and/or detection. Based on signals collected and received from the sensors, the control system may instruct one or more environmental units to adjust, calibrate, or maintain an optimal or predetermined environmental range. For example, if a temperature measured by a sensor is lower than an optimal temperature range, the control system may activate or adjust an environmental unit (e.g., heating or cooling element) to increase the temperature. In another example, to facilitate optimal reagent dispensing, an interferometer, as described elsewhere herein, may be used to determine a fluid layer thickness. The control system may, based on such determination, adjust, calibrate, or maintain dispensing parameters (e.g., fluid flow rate, substrate rotation rate, etc.). In another example, to facilitate efficient detection, pressure, distance, and/or positional sensors may be coupled to or integrated to the objective enclosure and/or the detector to provide feedback on efficiency and alignment of the objective. Based on signals collected and received from the sensors, the control system may adjust, calibrate, or maintain detection parameters (e.g., immersion fluid provision rate, alignment, movement speed, etc.). In some cases, optical signals collected by the detector itself may be used to calibrate the detection parameters, by the control system, for more efficient, accurate, and/or precise output.

Provided herein is a system for processing an analyte comprising one or more stations described herein, and one or more processors, individually or collectively programmed to, within at most 25 hours of running time of the processing station, output at least about 1.5 giga reads per substrate. Alternatively or additionally, the output may be at least about 0.5, 1.0, 2.0, 3.0, 4.0, 5.0, 6.0, 7.0, 8.0, 9.0, 10.0, 20.0, 30.0, 40.0, 50.0, 100.0, 200, 500, or 1000 giga reads per substrate or more. Alternatively or additionally, the one or more processors can, individually or collectively programmed to, within at most 25 hours of running time of the processing station, output at least 140 base pairs (bp) read length. Alternatively or additionally, the output may be at least about 100, 110, 120, 130, 140, 150, 200, 250, 300, 350, 400, 450, 500 bp read length or more. In some embodiments, the one or more processors are configured to output sequence reads of an average length longer than 500 bp, such as up to 550 bp, 600 bp, 700 bp, 800 bp, 900 bp, or up to 1000 bp or longer.

Alternatively or additionally, the one or more processors can, individually or collectively programmed to, within at most 25 hours of running time of the processing station, output at least 0.2 terabase reads per run. Alternatively or additionally, the output may include at least about 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 10.0, 20.0, 50.0, 100, 200, 300, 400, 500, 600, 700, 800, 900, or 1000 tera bases of sequence read information per run or more.

Provided herein is a system for processing an analyte comprising one or more stations described herein, and one or more processors, individually or collectively programmed to, within at most 15 hours of running time of the processing station, output at least about 1.5 giga reads per substrate. Alternatively or additionally, the output may be at least about 1.0, 2.0, 3.0, 4.0, 5.0, 6.0, 7.0, 8.0, 9.0, 10.0, 20.0, 30.0, 40.0, 50.0, 100.0, 200, 500, or 1000 giga reads per substrate or more. Alternatively or additionally, the one or more processors can, individually or collectively programmed to, within at most 15 hours of running time of the processing station, output at least 140 base pairs (bp) read length. Alternatively or additionally, the output may be at least about 100, 110, 120, 130, 140, 150, 200, 250, 300, 350, 400, 450, 500 bp read length or more. In some embodiments, the one or more processors are configured to output sequence reads of an average length longer than 500 bp, such as up to 550 bp, 600 bp, 700 bp, 800 bp, 900 bp, or up to 1000 bp or longer. Alternatively or additionally, the one or more processors can, individually or collectively programmed to, within at most 15 hours of running time of the processing station, output at least 0.2 terabase reads per run. Alternatively or additionally, the output may be at least about 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 10.0, 20.0, 50.0, 100, 200, 300, 400, 500, 600, 700, 800, 900, or 1000 tera bases of sequence read information per run or more.

In some examples, the methods may comprise discharging the output. In some cases, a portion of the output, such as at least about 1%, 2%, 5%, 10%, 15%, 20%, 30%, 40%, 50%, 60%, 70%, 80%, 90%, 99%, or more of the output may flow to the drain. In some examples, the method may comprise recycling one or more compounds from the output. Recycling may be performed for a number of reasons. For example, if a compound present in the output is a reagent, the compound may be recycled to its value. For example, a valuable or expensive reagent may be recycled. In some cases, a compound which may be harmful to the environment or otherwise not suitable for draining or discharging may be separated from the output. In some example, the waste generated from the methods and systems may be treated. Waste or output treatment may comprise PH neutralization, separation of given compounds from the output, or adjusting other parameters or characteristics of the waste. In some examples, components which may be inappropriate to drain, discharge, or otherwise discard and/or that are more economical to recycle off-site can be collected in a container. In some cases, the reagents may be shipped in a concentrated form to a facility or place for separation, storage, recycling, re-processing or other applications.

As will be appreciated, the systems, methods, and apparatus described herein may also have non-biological applications, such as for analyzing non-biological samples.

### Nucleic Acid Sequencing

The methods and systems provided herein may be useful in analyzing a nucleic acid molecule (e.g., a template nucleic acid molecule) using nucleic acid sequencing. Processing of a template nucleic acid molecule may be performed using a substrate comprising an array having immobilized thereto the template nucleic acid molecule (e.g., as described herein). The template nucleic acid molecule may be a sample nucleic acid molecule derived from a nucleic acid sample (e.g., as described herein). The template nucleic acid molecule may be immobilized to the substrate via a particle (e.g., bead). The template nucleic acid molecule may be hybridized to a growing nucleic acid strand. The substrate may be configured to rotate with respect to a central axis. A reagent solution comprising a plurality of nucleotides or nucleotide analogs may be directed across the array during rotation of the substrate. The plurality of nucleotides or nucleotide analogs may comprise non-terminated nucleotides to facilitate sequencing of homopolymeric regions of a template nucleic acid molecule. The plurality of nucleotides or nucleotide analogs may comprise a plurality of labeled nucleotides or nucleotide analogs labeled with an optically detectable label such as a fluorescent label (e.g., coupled to a nucleotide or nucleotide analog via a linker, such as a semi-rigid linker comprising a cleavable moiety). The plurality of nucleotides or nucleotide analogs may comprise nucleotides or nucleotide analogs of a single canonical type (e.g., adenine, uracil, thymine, cytosine, or guanine-containing nucleotides or nucleotide analogs) or of one or more different types. The template nucleic acid molecule may be subjected to conditions sufficient for nucleotides or nucleotide analogs of the plurality of nucleotides or nucleotide analogs to be incorporated into the growing nucleic acid strand (e.g., in a primer extension reaction). A signal (e.g., an optical signal) indicative of incorporation of a nucleotide or nucleotide analog may be detected (e.g., via optical detection, as described herein), thereby sequencing the nucleic acid molecule. The plurality of nucleotides or nucleotide analogs may be provided in a first reaction mixture, and provision of the first reaction mixture may be followed by one or more additional flows to wash away unbound nucleotides or nucleotide analogs and reagents, to cleave cleavable moieties of linkers coupling labels to nucleotides or nucleotide analogs, etc. Additional reaction mixtures comprising different combinations of nucleotides or nucleotide analogs may be provided (e.g., in a predefined sequence) to continue sequencing of the template nucleic acid molecule.

In another example, processing of a template nucleic acid molecule may be performed using an open substrate comprising an array of immobilized analytes thereon. For example, a template nucleic acid molecule may be immobilized to the open substrate via a particle (e.g., bead). The template nucleic acid molecule may be hybridized to a growing nucleic acid strand. The open substrate may be configured to rotate with respect to a central axis. A solution comprising a plurality of probes (e.g., nucleotides or nucleotide analogs) may be delivered to a region proximal to the central axis of the open substrate to introduce the solution to the open substrate. The solution may be dispersed across the open substrate such that at least one of the plurality of probes binds to at least one of the immobilized analytes to form a bound probe. Where the plurality of probes is a plurality of nucleotides or nucleotide analogs, the plurality of nucleotides or nucleotide analogs may comprise non-terminated nucleotides to facilitate sequencing of homopolymeric regions of a template nucleic acid molecule. The plurality of nucleotides or nucleotide analogs may comprise a plurality of labeled nucleotides or nucleotide analogs labeled with a fluorescent label (e.g., coupled to a nucleotide or nucleotide analog via a linker, such as a semi-rigid linker comprising a cleavable moiety). The plurality of nucleotides or nucleotide analogs may comprise nucleotides or nucleotide analogs of a single canonical type (e.g., adenine, uracil, thymine, cytosine, or guanine-containing nucleotides or nucleotide analogs) or of one or more different types. A bound probe may comprise a growing nucleic acid strand having a nucleotide or nucleotide analog incorporated therein. Formation of the bound probe may comprise subjecting a template nucleic acid molecule to conditions sufficient for nucleotides or nucleotide analogs of the plurality of nucleotides or nucleotide analogs to be incorporated into the growing nucleic acid strand (e.g., in a primer extension reaction). A first detector may be used to perform a first scan of the open substrate along a first set of scan paths and a second detector may be used to perform a second scan of the open substrate along a second set of scan paths. The first and second scans may be performed in sequence. Alternatively, the first and second scans may be performed simultaneously. The first and second scan paths may be linear paths along the open substrate. Alternatively, the first and second scan paths may be circular or spiral paths. The first and second scan paths may overlap. Alternatively, the first and second scan paths may not overlap. The first and second scan paths may be at least partially adjacent to one another. The first and second detectors may be of the same or different types. For example, the first and second detectors may both be optical detectors. The first and second detectors may be configured to detect signals (e.g., optical signals) indicative of formation of a bound probe (e.g., incorporation of a nucleotide or nucleotide analog into a growing nucleic acid strand). Accordingly, the first and second detectors may be used in sequencing template nucleic acid molecules. The plurality of probes (e.g., nucleotides or nucleotide analogs) may be provided in a first reaction mixture, and provision of the first reaction mixture may be followed by one or more additional flows to wash away unbound probes and reagents, to cleave cleavable moieties of linkers coupling labels to nucleotides or nucleotide analogs, etc. Additional reaction mixtures comprising different combinations of probes (e.g., nucleotides or nucleotide analogs) may be provided (e.g., in a predefined sequence) to, e.g., continue sequencing of the template nucleic acid molecule.

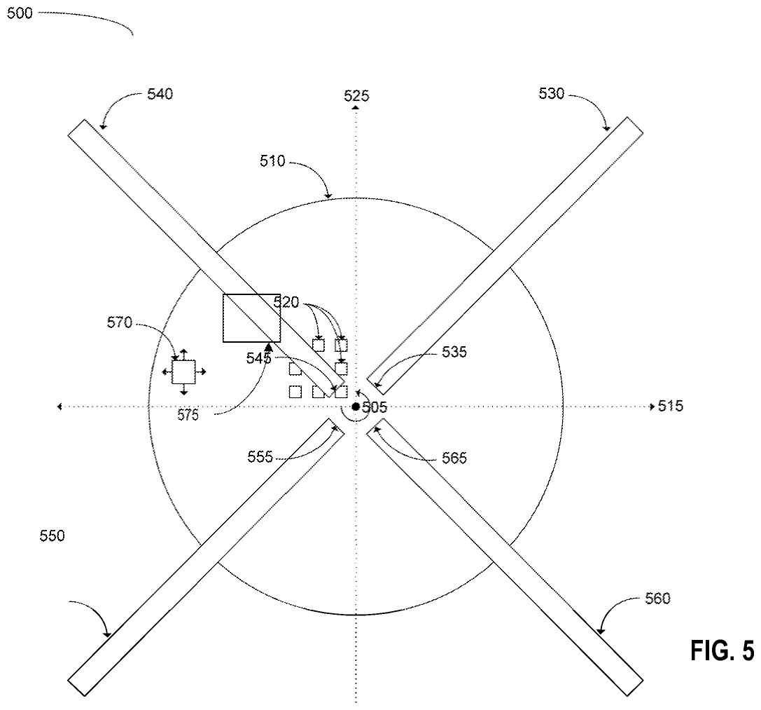

FIG. 5 shows a system 500 for sequencing a nucleic acid molecule or processing an analyte. The system may comprise a substrate 510. The substrate may comprise an array (e.g., arrays as illustrated in FIG. 2). The substrate may be open. The array may comprise one or more locations 520 configured to immobilize one or more nucleic acid molecules or analytes. The array may comprise a plurality of individually addressable locations. The array may comprise a linker (e.g., any binder described herein) that is coupled to a nucleic acid molecule to be sequenced. Alternatively or in combination, the nucleic acid molecule to be sequenced may be coupled to a particle. The particle (e.g., bead) may be immobilized to the array. The array may be textured. The array may be a patterned array. The array may be planar.

The substrate may be configured to rotate with respect to an axis 505. The axis may be an axis through the center of the substrate. The axis may be an off-center axis. The substrate may be configured to rotate at any useful rotational velocity. The substrate may be configured to undergo a change in relative position with respect to first or second longitudinal axes 515 and 525. For instance, the substrate may be translatable along the first and/or second longitudinal axes (as shown in FIG. 5). Alternatively, the substrate may be stationary along the first and/or second longitudinal axes. Alternatively or in combination, the substrate may be translatable along the axis. Alternatively or in combination, the substrate may be stationary along the axis. The relative position of the substrate may be configured to alternate between two or more positions (e.g., two or more positions with respect to an axis or a fluid channel as described herein). The first or second longitudinal axes may be substantially perpendicular, substantially parallel, or coincident with the axis.

The system may comprise one or more fluid channels 530, 540, 550, and 560. A fluid channel may comprise an inlet or outlet port (535, 545, 555, and 565) that may be a nozzle. A fluid channel may be configured to dispense a fluid (e.g., a solution comprising a plurality of probes, such as a plurality of nucleotides or nucleotide analogs) to the array. A fluid outlet port may be external to and may not contact the substrate. The relative position of one or more of the first, second, third, and fourth fluid channels may be configured to alternate between positions with respect to one or more of the longitudinal axes or the axis. For instance, the relative position of any of the first, second, third, or fourth fluid channel may be configured to alternate between a first position and a second position (e.g., by moving such channel, by moving the substrate, or by moving the channel and the substrate). Different fluid channels may be used to provide different combinations of probes and/or reagents to the array at the same or different times. For instance, a first fluid may comprise a first type of nucleotide or nucleotide mixture and a second fluid may comprise a second type of nucleotide or nucleotide mixture, where the first type or nucleotide or nucleotide mixture and the second type of nucleotide or nucleotide mixture differ from one another. Beneficially, where the first and second fluids comprise different types of reagents, each of the different reagents may remain free of contamination from the other reagents during dispensing. Alternatively, different fluid channels may be used to provide the same type of fluid through multiple fluid outlet ports (e.g., to increase coating speed). In some cases, a first fluid channel may be used to provide a nucleotide mixture and a second fluid channel may be used to provide a wash mixture. While four fluid channels and corresponding fluid outlet ports are shown in FIG. 5, any useful number of fluid channels (e.g., 1, 2, 3, 4, 5, 6, or more fluid channels) may be used. In some cases, a fluid channel may be configured to receive fluid from the substrate. Such a fluid channel may comprise a fluid inlet port disposed at the periphery of the substrate. The system may be configured to provide a fluid to the array during rotation of the substrate. Fluid may be dispensed to the array from different fluid channels at the same or different times while the substrate rotates at the same or different speeds and/or while the substrate remains stationary. When a fluid is dispensed during rotation of the substrate, the fluid may be dispensed across the substrate away from the central axis via centrifugal force. Additional details of systems useful for processing nucleic acid molecules that have undergone treated as described herein can be found in, for example, WO2019099886 and WO2020/186243, which are herein incorporated by reference in their entireties.

Sequencing a nucleic acid molecule (e.g., a nucleic acid molecule immobilized to a particle, which particle may be immobilized to a substrate as described herein) may comprise providing a solution comprising a plurality of optically (e.g., fluorescently) labeled nucleotides, where each optically (e.g., fluorescently) labeled nucleotide of the plurality of optically (e.g., fluorescently) labeled nucleotides is of a same type. The solution may also comprise a plurality of non-labeled nucleotides, which non-labeled nucleotides may comprise a nucleobase of the same type as that of the labeled nucleotides. The non-labeled and labeled nucleotides may be included in any useful ratio. For example, at least about 1%, about 2%, about 2.5%, about 5%, about 10%, about 20%, about 30%, about 40%, about 50%, about 60%, about 70%, about 80%, about 90%, about 95%, or more nucleotides in the solution may be fluorescently labeled. Alternatively, at most about 1%, about 2%, about 2.5%, about 5%, about 10%, about 20%, about 30%, about 40%, about 50%, about 60%, about 70%, about 80%, about 90%, about 95%, or fewer nucleotides in the solution may be fluorescently labeled. Labeled and/or non-labeled nucleotides may be non-terminated such that multiple nucleotides may be incorporated into a growing nucleic acid strand in sequence. A given optically (e.g., fluorescently) labeled nucleotide of the plurality of fluorescently labeled nucleotides may comprise an optical (e.g., fluorescent) dye that is connected to a nucleotide via a semi-rigid linker. Examples of linkers that may be used to link an optically detectable moiety to a nucleotide can be found in, for example, International Patent Application No. WO2020/172197, which is herein incorporated by reference in its entirety. The nucleic acid molecule (e.g., nucleic acid molecule coupled to a particle immobilized to a substrate) may be contacted with a primer under conditions sufficient to hybridize the primer to a nucleic acid molecule to be sequenced to generate a sequencing template. The sequencing template may then be contacted with a polymerase and the solution containing the plurality of optically (e.g., fluorescently) labeled nucleotides, wherein an optically (e.g., fluorescently) labeled nucleotide of the plurality of optically (e.g., fluorescently) labeled nucleotides is complementary to the nucleic acid molecule to be sequenced at a position adjacent to the primer. A substrate to which the sequencing template is coupled (e.g., via a particle immobilized to the substrate) may be rotated during provision of the solution such that the solution is radially dispersed across the substrate (e.g., as described herein). One or more optically (e.g., fluorescently) labeled nucleotides of the plurality of optically (e.g., fluorescently) labeled nucleotides may thus be incorporated into the sequencing template. One or more non-labeled nucleotides may also be incorporated (e.g., in a homopolymeric sequence). The solution comprising the plurality of optically (e.g., fluorescently) labeled nucleotides may be washed away from the sequencing template (e.g., using a wash solution). An optical (e.g., fluorescent) signal emitted by the sequencing template may then be measured. An optical (e.g., fluorescent) label may be cleaved from an incorporated labeled nucleotide after measuring the optical (e.g., fluorescent) signal (e.g., as described herein). Cleaving an optical (e.g., fluorescent) label may leave behind a scar (e.g., a residual chemical moiety). A washing flow may be used to remove cleaved labels and other residual materials. One or more additional nucleotide flows, such as one or more additional flows comprising nucleotides containing a same canonical type, may be used to ensure that nucleotides are incorporated into a substantial fraction of available positions. The process may then be repeated with an additional solution comprising additional nucleotides, such as nucleotides of a different type.

In some cases, a first solution used in a sequencing assay may include nucleotides of different types (e.g., comprising different canonical nucleobases), which nucleotides may comprise different fluorescent labels to facilitate differentiation between incorporation of different nucleotide types. Alternatively, the initial solution may include nucleotides of a same type (e.g., comprising only one type of nucleobase, such as only one type of canonical nucleobase). In an example, a sequencing assay may use four distinct four nucleotide flows including different canonical nucleobases that may be repeated in cyclical fashion (e.g., cycle 1: A, G, C, U; cycle 2 A, G, C, U; etc.). Each nucleotide flow may include nucleotides including nucleobases of a single canonical type (or analogs thereof), some of which may be include optical labeling reagents provided herein. The labeling fraction (e.g., % of nucleotides included in the flow that are attached to an optical labeling reagent) may be varied between, e.g., 0.5% to 100%. Labeling fractions may be different for different nucleotide flows. Nucleotides may not be terminated to facilitate incorporation into homopolymeric regions. The template may be contacted with a nucleotide flow, which may be followed by one or more wash flows (e.g., as described herein). The template may also be contacted with a cleavage flow (e.g., as described herein) including a cleavage reagent configured to cleave a portion of the optical labeling reagents attached to labeled nucleotides incorporated into the growing nucleic acid strand. A wash flow may be used to remove cleavage reagent and prepare the template for contact with a subsequent nucleotide flow. Emission may be detected from labeled nucleotides incorporated into the growing nucleic acid strand after each nucleotide flow.

The sequencing methods described herein may be applied for a single nucleic acid molecule, such as a single nucleic acid molecule immobilized to a single particle. The methods described herein may also be used to sequence a plurality of nucleic acid molecules, such as a plurality of nucleic acid molecules coupled to a plurality of particles, which plurality of particles may be immobilized to a substrate (e.g., as described herein). A substrate may comprise groupings of particles comprising nucleic acid molecules having common nucleic acid sequences (e.g., clonal populations.

### Computer Systems

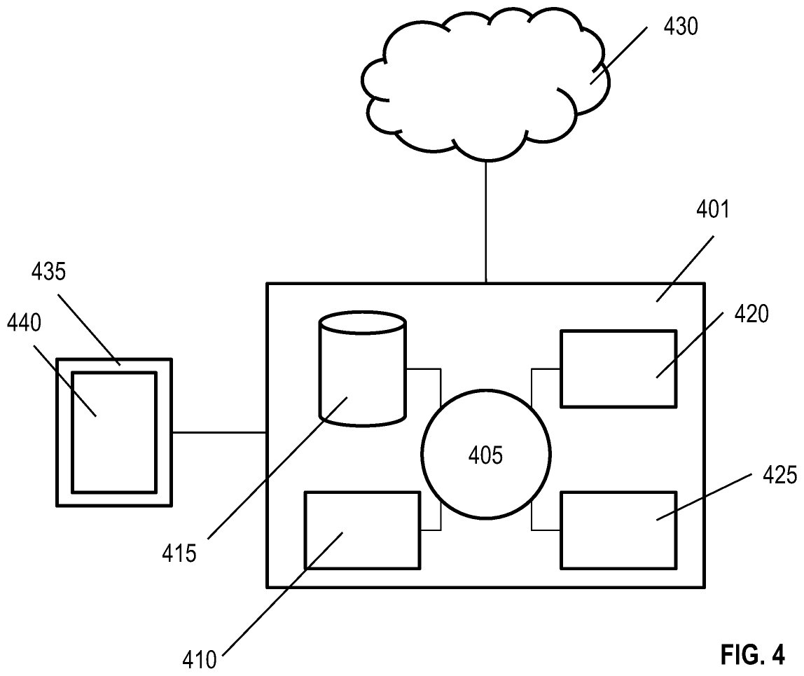

The present disclosure provides computer systems that are programmed to implement systems, methods, and apparatus of the disclosure. FIG. 4 shows a computer system 401 that is programmed or otherwise configured to process and/or detect a sample. The computer system 401 can regulate various aspects of methods and systems of the present disclosure. The computer system may be configured to regulate or communicate with any station, or component thereof, described herein. For example, the computer system 401 may comprise, or be, a controller configured to communicate with the user interface, fluid flow unit, other operating units, actuators, and/or detectors of the systems described herein. Alternatively, a controller may comprise the computer system 401.

The computer system 401 includes a central processing unit (CPU, also “processor” and “computer processor” herein) 405, which can be a single core or multi core processor, or a plurality of processors for parallel processing. The computer system 401 also includes memory or memory location 410 (e.g., random-access memory, read-only memory, flash memory), electronic storage unit 415 (e.g., hard disk), communication interface 420 (e.g., network adapter) for communicating with one or more other systems, and peripheral devices 425, such as cache, other memory, data storage and/or electronic display adapters. The memory 410, storage unit 415, interface 420 and peripheral devices 425 are in communication with the CPU 405 through a communication bus (solid lines), such as a motherboard. The storage unit 415 can be a data storage unit (or data repository) for storing data. The computer system 401 can be operatively coupled to a computer network (“network”) 430 with the aid of the communication interface 420. The network 430 can be the Internet, an internet and/or extranet, or an intranet and/or extranet that is in communication with the Internet. The network 430, in some cases, is a telecommunication and/or data network. The network 430 can include one or more computer servers, which can enable distributed computing, such as cloud computing. The network 430, in some cases with the aid of the computer system 401, can implement a peer-to-peer network, which may enable devices coupled to the computer system 401 to behave as a client or a server.

The CPU 405 can execute a sequence of machine-readable instructions, which can be embodied in a program or software. The instructions may be stored in a memory location, such as the memory 410. The instructions can be directed to the CPU 405, which can subsequently program or otherwise configure the CPU 405 to implement methods of the present disclosure. Examples of operations performed by the CPU 405 can include fetch, decode, execute, and writeback.

The CPU 405 can be part of a circuit, such as an integrated circuit. One or more other components of the system 401 can be included in the circuit. In some cases, the circuit is an application specific integrated circuit (ASIC).

The storage unit 415 can store files, such as drivers, libraries and saved programs. The storage unit 415 can store user data, e.g., user preferences and user programs. The computer system 401, in some cases, can include one or more additional data storage units that are external to the computer system 401, such as located on a remote server that is in communication with the computer system 401 through an intranet or the Internet.

The computer system 401 can communicate with one or more remote computer systems through the network 430. For instance, the computer system 401 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 401 via the network 430.

Methods as described herein can be implemented by way of machine (e.g., computer processor) executable code stored on an electronic storage location of the computer system 401, such as, for example, on the memory 410 or electronic storage unit 415. The machine executable or machine readable code can be provided in the form of software. During use, the code can be executed by the processor 405. In some cases, the code can be retrieved from the storage unit 415 and stored on the memory 410 for ready access by the processor 405. In some situations, the electronic storage unit 415 can be precluded, and machine-executable instructions are stored on memory 410.

The code can be pre-compiled and configured for use with a machine having a processer adapted to execute the code. Alternatively or additionally, the code can be compiled during runtime. The code can be supplied in a programming language that can be selected to enable the code to execute in a pre-compiled or as-compiled fashion.

Aspects of the systems and methods provided herein, such as the computer system 401, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 401 can include or be in communication with an electronic display 435 that comprises a user interface (UI) 440 for providing, for example, detection results to a user and/or receiving user input, such as user instructions. The UI may further present a console for configuring the fluid barrier systems, and/or components thereof (e.g., pressure-altering apparatus, environmental units, detectors, immersion enclosure, motion of detectors, motion of plates, motion of containers, motion of substrates, sample processing, etc.) of the present disclosure. Examples of UI's include, without limitation, a graphical user interface (GUI) and web-based user interface. The electronic display 435 may be part of or in communication with the instructions station 109, for example.

Methods and systems of the present disclosure can be implemented by way of one or more algorithms. An algorithm can be implemented by way of software upon execution by the central processing unit 405.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

### EMBODIMENTS

1. A method for sequencing a plurality of nucleic acid samples, the method comprising:
   (a) providing a nucleic acid sequencer having (i) a processing station configured to bring a nucleic acid molecule of a nucleic acid sample of said plurality of nucleic acid samples immobilized adjacent to a substrate into contact with a reagent to sequence said nucleic acid molecule; (ii) a sample station configured to supply said nucleic acid sample to said processing station; (iii) a substrate station configured to supply said substrate to said processing station, which substrate immobilizes adjacent thereto said nucleic acid sample; and (iv) a reagent station configured to supply said reagent to said processing station, wherein said reagent is supplied from a first reservoir or a second reservoir;
   (b) executing, by one or more processors individually or collectively, (i) at least a portion of a first queuing instruction to introduce a first set of one or more nucleic acid samples of said plurality of nucleic acid samples, including said nucleic acid sample, from said sample station to said processing station according to a first order of introduction defined by said first queuing instruction; (ii) a substrate loading instruction to introduce said substrate from said substrate station to said processing station and immobilize said first set of one or more nucleic acid samples adjacent to said substrate; and (iii) a sequencing instruction to draw said reagent from said first reservoir, from said second reservoir, or alternately from said first reservoir and said second reservoir and deliver said reagent to said processing station; and
   (c) while said processing station is in operation, performing one or more actions selected from the group consisting of:
      (1) introducing an additional nucleic acid sample to said sample station,
      (2) inputting a second queuing instruction and executing at least a portion of said second queuing instruction, wherein said second queuing instruction defines a second order of introduction that is different than said first order of introduction,
      (3) introducing an additional substrate to said substrate station, and
      (4) introducing an additional volume of said reagent to said reagent station by one or more of (i) replacing said first reservoir or said second reservoir with a third reservoir containing said reagent and (ii) replenishing said first reservoir or said second reservoir with said reagent.
2. The method of embodiment 1, wherein said processing station is configured to operate for at least 24 hours without human intervention.
3. The method of embodiment 2, wherein said processing station is configured to operate for at least 10 days without human intervention.
4. The method of any one of embodiments 1-3, wherein (c) comprises performing two or more actions selected from the group consisting of (1), (2), (3), and (4).
5. The method of any one of embodiments 1-3, wherein (c) comprises performing three or more actions selected from the group consisting of (1), (2), (3), and (4).
6. The method of any one of embodiments 1-3, wherein (c) comprises performing each of (1), (2), (3), and (4).
7. The method of any one of embodiments 1-6, wherein said sequencing instruction in (b)(iii) comprises instructions to draw said reagent from said first reservoir until said first reservoir is depleted below a predetermined threshold, then to draw said reagent from said second reservoir.
8. The method of any one of embodiments 1-7, wherein (4) comprises replacing or replenishing a reservoir from said first reservoir and said second reservoir that is depleted below a predetermined threshold.
9. The method of any one of embodiments 1-8, wherein said reagent comprises one or more members selected from the group consisting of a nucleotide solution, a cleaving solution, and a washing solution.
10. The method of embodiment 9, wherein said nucleotide solution comprises one or more members selected from the group consisting of adenine-containing nucleotides, cytosine-containing nucleotides, guanine-containing nucleotides, thymine-containing nucleotides, and uracil-containing nucleotides.
11. The method of embodiment 10, wherein said nucleotide solution comprises labeled nucleotides.
12. The method of any one of embodiments 1-11, wherein said substrate is a wafer.
13. The method of any one of embodiments 1-12, wherein said substrate comprises a substantially planar array.
14. The method of any one of embodiments 1-13, wherein said substrate comprises a plurality of independently addressable locations.
15. The method of any one of embodiments 1-14, wherein said substrate is configured to rotate about an axis in said processing station.
16. The method of any one of embodiments 1-15, wherein said substrate is configured to linearly translate in said processing station.
17. The method of any one of embodiments 1-16, wherein said nucleic acid molecule is coupled to a bead, wherein said bead is immobilized adjacent to said substrate.
18. The method of any one of embodiments 1-17, wherein a plurality of nucleic acid samples is immobilized adjacent to said substrate, wherein nucleic acid samples of said plurality of nucleic acid samples are from different sources.
19. The method of embodiment 18, wherein said plurality of nucleic acid samples is compatible with a common sequencing protocol.
20. The method of any one of embodiments 1-19, wherein said processing station is disposed in a first environment different from a second environment in which said sample station, substrate station, and/or reagent station is disposed.
21. The method of embodiment 20, wherein said first environment has a higher relative humidity than said second environment.
22. The method of embodiment 20 or 21, wherein said first environment comprises one or more regions of controlled average temperature different from a second average temperature of said second environment.
23. The method of any one of embodiments 1-22, wherein said processing station is disposed in an environment different from an ambient environment.
24. The method of embodiment 23, wherein said environment has a higher relative humidity than said ambient environment.
25. The method of embodiment 23 or 24, wherein said environment comprises one or more regions of controlled average temperature different from an ambient temperature.
26. The method of any one of embodiments 1-25, wherein said nucleic acid sequencer comprises a dilution station configured to dilute said reagent from said reagent station prior to delivery of said reagent to said processing station.
27. The method of embodiment 26, wherein said reagent is diluted with deionized water.
28. The method of any one of embodiments 1-27, wherein said substrate station comprises a sealed environment.
29. The method of embodiment 28, wherein said substrate station comprises a hermetically sealed environment.
30. The method of embodiment 28 or 29, wherein said substrate station comprise a vacuum desiccator.
31. The method of any one of embodiments 1-30, wherein said one or more processors are configured to, individually or collectively, within at most 40 hours of running time of said processing station, output one or more selected from the group consisting of:
   (i) at least 1.5 giga reads per substrate,
   (ii) sequence reads averaging at least 140 base pairs (bp) in length, and
   (iii) at least 0.2 terabase reads per run.
32. The method of embodiment 31, wherein said one or more processors are configured to, within at most 40 hours of running time of said processing station, output at least 40.0 Giga reads per substrate.
33. The method of embodiment 31 or 32, wherein said one or more processors are configured to, within at most 40 hours of running time of said processing station, output at least 500 bp read length.
34. The method of any one of embodiments 31-33, wherein said one or more processors are configured to, within at most 40 hours of running time of said processing station, output at least 6.5 terabase reads per run.
35. The method of any one of embodiments 1-30, wherein said one or more processors are configured to, within at most 25 hours of running time of said processing station, output one or more selected from the group consisting of:
   (i) at least 1.5 giga reads per substrate,
   (ii) sequence reads averaging at least 140 base pairs (bp) in length, and
   (iii) at least 0.2 terabase reads per run.
36. The method of any one of embodiments 1-30, wherein said one or more processors are configured to, within at most 15 hours of running time of said processing station, output one or more selected from the group consisting of:
   (i) at least 1.5 giga reads per substrate,
   (ii) sequence reads averaging at least 140 base pairs (bp) in length, and
   (iii) at least 0.2 terabase reads per run.
37. The method of any one of embodiments 1-36, further comprising (A) inputting (1) said plurality of nucleic acid samples, including said nucleic acid sample, to said sample station and (2) a plurality of substrates, including said substrate, to said substrate station; and (B) providing to said one or more processors user instructions to start two or more sequencing cycles.
38. The method of embodiment 37, further comprising (C) in a first sequencing cycle, processing a first nucleic acid sample from said plurality of nucleic acid samples on a first substrate of said plurality of substrates; and (D) during or subsequent to said first sequencing cycle, in a second sequencing cycle, processing a second nucleic acid sample from said plurality of nucleic acid samples on a second substrate of said plurality of substrates, wherein said second sequencing cycle is performed in absence of additional user intervention.
39. The method of embodiment 37 or 38, wherein said two or more sequencing cycles are at least 5 sequencing cycles.
40. The method of embodiment 39, wherein said two or more sequencing cycles are at least 10 sequencing cycles.
41. The method of embodiment 40, wherein said two or more sequencing cycles are at least 20 sequencing cycles.
42. The method of any one of embodiments 1-41, further comprising purifying a reagent mixture comprising said reagent prior to delivery of said reagent to said processing station, wherein said reagent mixture comprises a plurality of nucleotides or nucleotide analogs.
43. The method of embodiment 42, wherein said purifying comprises (A) directing said reagent mixture to a reaction space comprising a support having a first plurality of nucleic acid molecules immobilized adjacent thereto; (B) incorporating a subset of nucleotides or nucleotide analogs from said plurality of nucleotides or nucleotide analogs into said first plurality of nucleic acid molecules, thereby providing a remainder of said plurality of nucleotides or nucleotides analogs, wherein (B) is performed without detecting said subset of nucleotides incorporated into said plurality of nucleic acid molecules; and (C) delivering said remainder of said plurality of nucleotides or nucleotide analogs to said processing station.
44. The method of embodiment 43, further comprising (D) incorporating at least a subset of said remainder of said plurality of nucleotides or nucleotides analogs into a growing stand associated with said nucleic acid molecule.
45. The method of embodiment 43 or 44, wherein said subset of nucleotides or nucleotide analogs comprises less than 10%, less than 5%, less than 1%, less than 0.1%, or less than 0.01% of said plurality of nucleotides or nucleotide analogs.
46. The method of any one of embodiments 43-45, wherein said remainder of said plurality of nucleotides or nucleotide analogs has a ratio of a number of nucleotides or nucleotide analogs of one or more but less than all canonical types to a number of nucleotides or nucleotide analogs of all other canonical types which is greater than 19:1.
47. The method of embodiment 46, wherein said ratio is at least 29:1.
48. The method of embodiment 47, wherein said ratio is at least 99:1.
49. The method of embodiment 48, wherein said ratio is at least 999:1.
50. The method of embodiment 42, wherein said purifying comprises (A) selecting from a set of canonical types of nucleotides or nucleotides analogs a subset of canonical types of nucleotides or nucleotide analogs; (B) directing said reagent mixture to a reaction space comprising a support having a plurality of nucleic acid molecules immobilized thereto, wherein a percentage of nucleotides or nucleotide analogs corresponding to said subset relative to all other nucleotides or nucleotide analogs in said mixture is greater than 50%; and (C) incorporating nucleotides or nucleotide analogs from said mixture that do not correspond to said subset into said plurality of nucleic acid molecules such that said percentage is increased following said incorporating, wherein (A)-(C) are performed in absence of sequencing or sequence identification of said plurality of nucleic acid molecules.
51. The method of embodiment 42, wherein said purifying comprises (A) directing said reagent mixture to a reaction space comprising a support having a plurality of nucleic acid molecules immobilized thereto; (B) incorporating a subset of nucleotides or nucleotide analogs form said plurality of nucleotides or nucleotide analogs into said plurality of nucleic acid molecules, thereby providing a remainder of said plurality of nucleotides or nucleotides analogs, wherein (A)-(B) are performed in absence of sequencing or sequence identification of said plurality of nucleic acid molecules.
52. The method of embodiment 51, further comprising (C) using said remainder of said plurality of nucleotides or nucleotide analogs to perform nucleic acid sequencing by synthesis.
53. A system for sequencing a plurality of nucleic acid samples, comprising:
   a processing station configured to bring a nucleic acid molecule of a nucleic acid sample of said plurality of nucleic acid samples immobilized adjacent to a substrate into contact with a reagent to sequence the nucleic acid molecule;
   a sample station configured to supply said nucleic acid sample to said processing station;
   a substrate station configured to supply said substrate to said processing station, which substrate is configured to immobilize adjacent thereto said nucleic acid sample;
   a reagent station configured to supply said reagent to said processing station, wherein said reagent is supplied from a first reservoir or a second reservoir; and
   one or more processors, individually or collectively, programmed to execute (i) at least a portion of a first queuing instruction to introduce a first set of one or more nucleic acid samples of said plurality of nucleic acid samples, including said nucleic acid sample, from said sample station to said processing station according to a first order of introduction defined by said first queuing instruction, (ii) a substrate loading instruction to introduce said substrate from said substrate station to said processing station and immobilize said first set of one or more nucleic acid samples adjacent to said substrate, and (iii) a sequencing instruction to draw said reagent from said first reservoir, from said second reservoir, or alternately from said first reservoir and said second reservoir and deliver said reagent to said processing station,
   wherein said processing station is capable of operating during performance of one or more actions selected from the group consisting of:
      (1) introducing an additional nucleic acid sample of said plurality of nucleic acid samples to said sample station,
      (2) inputting a second queuing instruction and executing at least a portion of said second queuing instruction, wherein said second queuing instruction defines a second order of introduction that is different than said first order of introduction,
      (3) introducing an additional substrate to said substrate station, and
      (4) introducing an additional volume of said reagent to said reagent station by one or more (i) replacing said first reservoir or said second reservoir with a third reservoir containing said reagent and (ii) replenishing said first reservoir or said second reservoir with said reagent.
54. The system of embodiment 53, wherein said processing station is capable of operating for at least 24 hours without human intervention.
55. The system of embodiment 54, wherein said sequencing system is capable of continuous operation for more than 10 days with human intervention at intervals of not less than 18 hours.
56. The system of any one of embodiments 53-56, wherein said processing station is capable of operating during the performance of two or more actions selected from the group consisting of (1), (2), (3), and (4).
57. The system of any one of embodiments 53-56, wherein said processing station is capable of operating during the performance of three or more actions selected from the group consisting of (1), (2), (3), and (4).
58. The system of any one of embodiments 53-56, wherein said processing station is capable of operating during the performance of each of (1), (2), (3), and (4).
59. The system of any one of embodiments 53-58, wherein said sequencing instruction comprises instructions to draw said reagent from said first reservoir until said first reservoir is depleted below a predetermined threshold, then to draw said reagent from said second reservoir.
60. The system of any one of embodiments 53-59, wherein (4) comprises replacing or replenishing a reservoir from said first reservoir and said second reservoir that is depleted below a predetermined threshold.
61. The system of any one of embodiments 53-60, wherein said reagent comprises one or more members selected from the group consisting of a nucleotide solution, a cleavage solution, and a washing solution.
62. The system of embodiment 61, wherein said nucleotide solution comprises one or more members selected from the group consisting of adenine-containing nucleotides, cytosine-containing nucleotides, guanine-containing nucleotides, thymine-containing nucleotides, and uracil-containing nucleotides.
63. The system of embodiment 62, wherein said nucleotide solution comprises labeled nucleotides.
64. The system of any one of embodiments 53-63, wherein said substrate is a wafer.
65. The system of any one of embodiments 53-64, wherein said substrate comprises a substantially planar array.
66. The system of any one of embodiments 53-65, wherein said substrate comprises a plurality of independently addressable locations.
67. The system of any one of embodiments 53-66, wherein said substrate is configured to rotate about an axis in said processing station.
68. The system of any one of embodiments 53-67, wherein said substrate is configured to linearly translate in said processing station.
69. The system of any one of embodiments 53-68, wherein said nucleic acid molecule is coupled to a bead, wherein said bead is immobilized adjacent to said substrate.
70. The system of any one of embodiments 53-69, wherein a plurality of nucleic acid samples is immobilized adjacent to said substrate, wherein nucleic acid samples of said plurality of nucleic acid samples are from different sources.
71. The system of embodiment 70, wherein said plurality of nucleic acid samples is compatible with a common sequencing protocol.
72. The system of any one of embodiments 53-71, wherein said processing station is disposed in a first environment different from a second environment in which said sample station, substrate station, and/or reagent station is disposed.
73. The system of embodiment 72, wherein said first environment has a higher relative humidity than said second environment.
74. The system of embodiment 72 or 73, wherein said first environment comprises one or more regions of controlled average temperature different from a second average temperature of said second environment.
75. The system of any one of embodiments 53-74, wherein said processing station is disposed in an environment different from an ambient environment.
76. The system of embodiment 75, wherein said environment has a higher relative humidity than said ambient environment.
77. The system of embodiment 75 or 76, wherein said environment comprises one or more regions of controlled average temperature different from an ambient temperature.
78. The system of any one of embodiments 53-77, wherein said nucleic acid sequencer comprises a dilution station configured to dilute said reagent from said reagent station prior to delivery of said reagent to said processing station.
79. The system of embodiment 78, wherein said reagent is diluted with deionized water.
80. The system of any one of embodiments 53-79, wherein said substrate station comprises a sealed environment.
81. The system of embodiment 80, wherein said substrate station comprises a hermetically sealed environment.
82. The system of embodiment 80 or 81, wherein said substrate station comprise a vacuum desiccator.
83. The system of any one of embodiments 53-82, wherein said one or more processors are configured to, individually or collectively, within at most 40 hours of running time of said processing station, output one or more selected from the group consisting of:
   (i) at least 1.5 giga reads per substrate,
   (ii) sequence reads averaging at least 140 base pairs (bp) in length, and
   (iii) at least 0.2 terabase reads per run.
84. The system of embodiment 83, wherein said one or more processors are configured to, within at most 40 hours of running time of said processing station, output at least 40.0 Giga reads per substrate.
85. The system of embodiment 83 or 84, wherein said one or more processors are configured to, within at most 40 hours of running time of said processing station, output at least 500 bp read length.
86. The system of any one of embodiments 83-85, wherein said one or more processors are configured to, within at most 40 hours of running time of said processing station, output at least 6.5 terabase reads per run.
87. The system of any one of embodiments 53-86, wherein said one or more processors are configured to, within at most 25 hours of running time of said processing station, output one or more selected from the group consisting of:
   (i) at least 1.5 giga reads per substrate,
   (ii) sequence reads averaging at least 140 base pairs (bp) in length, and
   (iii) at least 0.2 terabase reads per run.
88. The system of any one of embodiments 53-87, wherein said one or more processors are configured to, within at most 15 hours of running time of said processing station, output one or more selected from the group consisting of:
   (i) at least 1.5 giga reads per substrate,
   (ii) sequence reads averaging at least 140 base pairs (bp) in length, and
   (iii) at least 0.2 terabase reads per run.
89. The system of any one of embodiments 53-88, wherein said system is further configured to (A) input (1) said plurality of nucleic acid samples, including said nucleic acid sample, to said sample station and (2) a plurality of substrates, including said substrate, to said substrate station; and (B) provide to said one or more processors user instructions to start two or more sequencing cycles.
90. The system of embodiment 89, wherein said system is further configured to (C) in a first sequencing cycle, process a first nucleic acid sample from said plurality of nucleic acid samples on a first substrate of said plurality of substrates; and (D) during or subsequent to said first sequencing cycle, in a second sequencing cycle, process a second nucleic acid sample from said plurality of nucleic acid samples on a second substrate of said plurality of substrates, wherein said second sequencing cycle is configured to be performed in absence of additional user intervention.
91. The system of embodiment 89 or 90, wherein said two or more sequencing cycles are at least 5 sequencing cycles.
92. The system of embodiment 91, wherein said two or more sequencing cycles are at least 10 sequencing cycles.
93. The system of embodiment 92, wherein said two or more sequencing cycles are at least 20 sequencing cycles.
94. The system of any one of embodiments 53-93, wherein said system is further configured to purify a reagent mixture comprising said reagent prior to delivery of said reagent to said processing station, wherein said reagent mixture comprises a plurality of nucleotides or nucleotide analogs.
95. The system of embodiment 94, wherein said system is configured to (A) direct said reagent mixture to a reaction space comprising a support having a first plurality of nucleic acid molecules immobilized adjacent thereto; (B) incorporate a subset of nucleotides or nucleotide analogs from said plurality of nucleotides or nucleotide analogs into said first plurality of nucleic acid molecules, thereby providing a remainder of said plurality of nucleotides or nucleotides analogs, wherein (B) is configured to be performed without detecting said subset of nucleotides incorporated into said plurality of nucleic acid molecules; and (C) deliver said remainder of said plurality of nucleotides or nucleotide analogs to said processing station.
96. The system of embodiment 95, wherein said system is further configured to (D) incorporate at least a subset of said remainder of said plurality of nucleotides or nucleotides analogs into a growing stand associated with said nucleic acid molecule.
97. The system of embodiment 95 or 96, wherein said subset of nucleotides or nucleotide analogs comprises less than 10%, less than 5%, less than 1%, less than 0.1%, or less than 0.01% of said plurality of nucleotides or nucleotide analogs.
98. The system of any one of embodiments 95-97, wherein said remainder of said plurality of nucleotides or nucleotide analogs has a ratio of a number of nucleotides or nucleotide analogs of one or more but less than all canonical types to a number of nucleotides or nucleotide analogs of all other canonical types which is greater than 19:1.
99. The system of embodiment 98, wherein said ratio is at least 29:1.
100. The system of embodiment 99, wherein said ratio is at least 99:1.
101. The system of embodiment 100, wherein said ratio is at least 999:1.
102. The system of embodiment 94, wherein said system is further configured to (A) select from a set of canonical types of nucleotides or nucleotides analogs a subset of canonical types of nucleotides or nucleotide analogs; (B) direct said reagent mixture to a reaction space comprising a support having a plurality of nucleic acid molecules immobilized thereto, wherein a percentage of nucleotides or nucleotide analogs corresponding to said subset relative to all other nucleotides or nucleotide analogs in said mixture is greater than 50%; and (C) incorporate nucleotides or nucleotide analogs from said mixture that do not correspond to said subset into said plurality of nucleic acid molecules such that said percentage is increased following said incorporating, wherein (A)-(C) are configured to be performed in absence of sequencing or sequence identification of said plurality of nucleic acid molecules.
103. The system of embodiment 94, wherein said system is further configured to (A) direct said reagent mixture to a reaction space comprising a support having a plurality of nucleic acid molecules immobilized thereto; (B) incorporate a subset of nucleotides or nucleotide analogs form said plurality of nucleotides or nucleotide analogs into said plurality of nucleic acid molecules, thereby providing a remainder of said plurality of nucleotides or nucleotides analogs, wherein (A)-(B) are configured to be performed in absence of sequencing or sequence identification of said plurality of nucleic acid molecules.
104. The system of embodiment 103, wherein said system is further configured to (C) use said remainder of said plurality of nucleotides or nucleotide analogs to perform nucleic acid sequencing by synthesis.
105. A method for processing analytes, comprising:
   (a) executing, by one or more processors individually or collectively, at least a portion of a first queuing instruction to introduce a first set of one or more sample analytes of a plurality of sample analytes from a sample station of a system into a processing station of said system according to a first order of introduction defined by said first queuing instruction, wherein said sample station comprises a plurality of sample sources, wherein each of said plurality of sample sources is accessible for introduction of sample analytes from said plurality of samples sources into said processing station by one or more actuators, and wherein said first queuing instruction defines said first order of introduction of said sample analytes between said plurality of sample sources;
   (b) receiving a second queuing instruction, wherein said second queuing instruction defines a second order of introduction different from said first order of introduction; and
   (c) executing, by said one or more processors individually or collectively, at least a portion of said second queuing instruction to introduce a second set of one or more sample analytes of said plurality of sample analytes from said sample station to said processing station according to said second order of introduction while said system is in operation.
106. The method of embodiment 105, wherein (c) is performed while said processing system is in operation.
107. The method of embodiment 105 or 106, wherein said executing in (c) is performed in absence of terminating said operation of said processing station.
108. The method of any one of embodiments 105-107, wherein, during said operation, said processing station is maintained at a different environment than an ambient environment.
109. The method of any one of embodiments 105-108, wherein, during said operation, said processing station is maintained at a different environment than an environment of said sample station.
110. The method of any one of embodiments 105-109, wherein, during said operation, said processing station is maintained at a different temperature than an ambient temperature.
111. The method of any one of embodiments 105-110, wherein, during said operation, said processing station is maintained at a different humidity than an ambient humidity.
112. The method of any one of embodiments 105-111, wherein said processing station is configured to direct a sample analyte from a sample source in said sample station onto a substrate in said processing station.
113. The method of embodiment 112, wherein said substrate is capable of processing a plurality of samples.
114. The method of embodiment 113, wherein a group of samples are selected according to a sample selection instruction based at least in part on use of area of said substrate.
115. The method of embodiment 113, wherein a group of samples are selected such that said group of samples can be processed using a first set of conditions which differs from a second set of conditions at which said other samples are processed.
116. The method of any one of embodiments 105-115, wherein said processing station is configured to direct a reagent to contact a sample analyte from a sample source in said sample station.
117. The method of any one of embodiments 105-116, wherein said processing station is configured to detect a signal associated with a sample analyte from a sample source in said sample station.
118. The method of any one of embodiments 105-117, further comprising, prior to (b), providing a new sample source in said sample station while said system is in operation.
119. The method of embodiment 118, wherein said new sample source is provided while said processing station is in operation.
120. The method of any one of embodiments 105-119, wherein said plurality of sample analytes comprises a plurality of nucleic acid molecules.
121. A method for processing analytes, comprising:
   (a) providing a first reagent source and a second reagent source in a reagent station, wherein each of said first reagent source and said second reagent source (i) comprises a first reagent, and (ii) is accessible for introduction of said first reagent from said reagent station to a processing station by a controller, wherein said processing station is configured to facilitate one or more operations using said first reagent;
   (b) directing said first reagent from said first reagent source to said processing station;
   (c) directing said first reagent from said second reagent source to said processing station;
   (d) while said processing station is in operation and receiving said first reagent from said second reagent source, (i) replacing said first reagent source with a third reagent source comprising said first reagent, wherein said third reagent source is accessible for introduction of said first reagent from said reagent station to said processing station by said controller, or (ii) replenishing said first reagent source with an additional volume of said first reagent; and
   (e) directing said first reagent from (i) said third reagent source, or (ii) said additional volume of said first reagent in said first reagent source, to said processing station.
122. The method of embodiment 121, wherein said controller is configured to control one or more actuators.
123. The method of embodiment 121 or 122, wherein said controller is configured to control one or more valves in fluid communication with said first reagent source or said second reagent source.
124. The method of any one of embodiments 121-123, wherein (c) is initiated when said first reagent source is depleted below a predetermined threshold level.
125. The method of embodiment 124, wherein said predetermined threshold level is a fully depleted level.
126. The method of any one of embodiments 121-125, wherein (e) is initiated when said second reagent source is depleted below a predetermined threshold level.
127. The method of embodiment 126, wherein said predetermined threshold level is a fully depleted level.
128. The method of any one of embodiments 121-127, wherein (i) said replacing or (ii) said replenishing in (d) is performed in absence of terminating said operation of said processing station.
129. The method of any one of embodiments 121-128, further comprising diluting said first reagent with a diluent subsequent to departure from said reagent station and prior to delivery to said processing station.
130. The method of embodiment 129, wherein said diluent is deionized water.
131. The method of embodiment 129 or 130, wherein said diluent is delivered from a diluent source comprising said diluent.
132. The method of any one of embodiments 129-131, wherein said diluent is produced within an enclosure comprising therein said reagent station and said processing station.
133. The method of any one of embodiments 121-132, wherein said directing said first reagent from said second reagent source in (c) commences subsequent to a volume of said first reagent in said first reagent source decreasing below a predetermined threshold.
134. The method of embodiment 133, wherein said directing in (e) commences subsequent to a volume of said first reagent in said second reagent source decreasing below a second predetermined threshold.
135. The method of embodiment 134, wherein said predetermined threshold and said second predetermined threshold are the same.
136. The method of any one of embodiments 121-135, wherein, during said operation, said processing station is maintained at a different environment than an ambient environment.
137. The method of any one of embodiments 121-136, wherein, during said operation, said processing station is maintained at a different environment than an environment of said reagent station.
138. The method of any one of embodiments 121-137, wherein, during said operation, said processing station is maintained at a different temperature than an ambient temperature.
139. The method of any one of embodiments 121-138, wherein, during said operation, said processing station is maintained at a different humidity than an ambient humidity.
140. The method of any one of embodiments 121-139, wherein said processing station is configured to direct said reagent to contact an analyte in said processing station.
141. The method of embodiment 140, wherein said processing station is configured to detect a signal associated with said analyte.
142. The method of embodiment 140 or 141, wherein said analyte is a nucleic acid molecule.
143. The method of any one of embodiments 121-142, wherein said first reagent source comprises a container.
144. The method of any one of embodiments 121-143, wherein said first reagent comprises a nucleotide solution, a washing solution, or a cleavage solution.
145. The method of embodiment 144, wherein said nucleotide solution comprises adenine-containing nucleotides, cytosine-containing nucleotides, guanine-containing nucleotides, thymine-containing nucleotides, or uracil-containing nucleotides.
146. The method of embodiment 144 or 145, wherein said nucleotide solution comprises labeled nucleotides.
147. The method of any one of embodiments 121-146, further comprising preparing said first reagent of said third reagent source or said additional volume of said first reagent source from a frozen concentrate.
148. The method of any one of embodiments 121-147, wherein said processing station is configured to operate for at least 24 hours without human intervention.
149. The method of embodiment 148, wherein said processing station is configured to operate for at least 40 hours without human intervention.
150. A method for processing analytes, comprising:
   (a) providing a plurality of substrates in a substrate station, wherein each of said plurality of substrates is accessible for introduction of substrates from said substrate station into a processing station of a system by one or more actuators;
   (b) delivering, by one or more actuators, a first substrate of said plurality of substrates into said processing station;
   (c) in said processing station, performing a process involving an analyte immobilized adjacent to said first substrate; and
   (d) delivering, by said one or more actuators, a second substrate of said plurality of substrates into said processing station while said system is in operation.
151. The method of embodiment 150, wherein said delivering in (d) is performed while said processing station is performing said process.
152. The method of embodiment 150 or 151, wherein said delivering in (d) is performed in absence of terminating said process of said processing station.
153. The method of any one of embodiments 150-152, wherein, during said process, said processing station is maintained at a different environment than an ambient environment.
154. The method of any one of embodiments 150-153, wherein, during said process, said processing station is maintained at a different environment than an environment of said substrate station.
155. The method of any one of embodiments 150-154, wherein, during said process, said processing station is maintained at a different temperature than an ambient temperature.
156. The method of any one of embodiments 150-155, wherein, during said process, said processing station is maintained at a different humidity than an ambient humidity.
157. The method of any one of embodiments 150-156, wherein said processing station is configured to perform processes on two or more substrates simultaneously.
158. The method of any one of embodiments 150-157, wherein said processing station is configured to deposit said analyte onto said first substrate.
159. The method of any one of embodiments 150-158, wherein said processing station is configured to direct a reagent to contact said analyte immobilized adjacent to said first substrate.
160. The method of embodiment 159, wherein said reagent comprises a nucleotide solution, a washing solution, or a cleavage solution.
161. The method of embodiment 160, wherein said nucleotide solution comprises adenine-containing nucleotides, cytosine-containing nucleotides, guanine-containing nucleotides, thymine-containing nucleotides, or uracil-containing nucleotides.
162. The method of embodiment 160 or 161, wherein said nucleotide solution comprises labeled nucleotides.
163. The method of any one of embodiments 150-162, wherein said processing station is configured to detect a signal associated with said analyte.
164. The method of embodiment 163, wherein said signal is a fluorescent signal.
165. The method of any one of embodiments 150-164, wherein said analyte is a nucleic acid molecule.
166. The method of any one of embodiments 150-165, wherein said plurality of substrates is a plurality of wafers.
167. The method of any one of embodiments 150-166, wherein said first substrate is substantially planar.
168. The method of any one of embodiments 150-167, wherein said first substrate is not a flow cell.
169. The method of any one of embodiments 150-168, wherein said first substrate is patterned or textured.
170. The method of any one of embodiments 150-169, wherein said substrate station comprises a rack containing said plurality of substrates.
171. The method of embodiment 170, wherein said rack is a vertical rack that contains said plurality of substrates in a substantially horizontal position.
172. The method of embodiment 170, wherein said rack is a horizontal rack that contains said plurality of substrates in a substantially vertical position.
173. The method of any one of embodiments 150-172, wherein said first substrate is delivered to a first location of said processing station and said second substrate is delivered to a second location of said processing station that is different than said first location.
174. The method of embodiment 173, wherein said second location is disposed below said first location.
175. The method of embodiment 173, wherein said second location is adjacent to said first location.
176. The method of any one of embodiments 173-175, further comprising removing said first substrate from said first location of said processing station.
177. The method of any one of embodiments 173-176, further comprising delivering said second substrate to said first location of said processing station.
178. The method of any one of embodiments 150-177, wherein said processing station is configured to operate for at least 24 hours without human intervention.
179. The method of embodiment 178, wherein said processing station is configured to operate for at least 40 hours without human intervention.
180. A method for processing analytes, comprising:
   (a) inputting (1) a plurality of nucleic acid samples from different sample sources, and (2) a plurality of substrates;
   (b) providing, to one or more processors, user instructions to start two or more sequencing cycles;
   (c) in a first sequencing cycle, processing a first nucleic acid sample of said plurality of nucleic acid samples on a first substrate of said plurality of substrates; and
   (d) during or subsequent to said first sequencing cycle, in a second sequencing cycle, processing a second nucleic acid sample of said plurality of nucleic acid samples on a second substrate of said plurality of substrates, wherein said second sequencing cycle is performed in absence of additional user intervention.
181. The method of embodiment 180, further comprising, during or subsequent to an (n−1)th sequencing cycle, in an nth sequencing cycle, processing an nth nucleic acid sample from said plurality of nucleic acid samples on an nth substrate of said plurality of substrates, wherein said nth sequencing cycle is performed in absence of additional user instructions from said user instructions.
182. The method of embodiment 180 or 181, wherein said plurality of substrates is a plurality of wafers.
183. The method of any one of embodiments 180-182, wherein first substrate or said second substrate is substantially planar.
184. The method of any one of embodiments 180-183, wherein said first substrate and said second substrate are not flow cells.
185. The method of any one of embodiments 180-184, wherein said first substrate or said second substrate is textured or patterned.
186. The method of any one of embodiments 180-185, wherein said first nucleic acid sample comprises a first plurality of nucleic acid molecules and said second nucleic acid sample comprises a second plurality of nucleic acid molecules.
187. The method of any one of embodiments 180-186, further comprising depositing said first nucleic acid sample onto said first substrate and depositing said second nucleic acid sample onto said second substrate.
188. The method of any one of embodiments 180-187, wherein said first nucleic acid sample is immobilized adjacent to said first substrate and said second nucleic acid sample is immobilized adjacent to said second substrate.
189. The method of embodiment 188, wherein said first nucleic acid sample is immobilized to said first substrate via a first plurality of particles and said second nucleic acid sample is immobilized to said second substrate via a second plurality of particles.
190. The method of any one of embodiments 180-189, wherein said first sequencing cycle comprises directing, in sequence, a first set of reagents, a second set of reagents, a third set of reagents, and a fourth set of reagents to said first nucleic acid sample.
191. The method of embodiment 190, wherein each of said first set of reagents, said second set of reagents, said third set of reagents, and said fourth set of reagents comprises a washing solution.
192. The method of embodiment 190 and 191, wherein each of said first set of reagents, said second set of reagents, said third set of reagents, and said fourth set of reagents comprises a nucleotide solution.
193. The method of embodiment 192, wherein said nucleotide solutions of said first set of reagents, said second set of reagents, said third set of reagents, and said fourth set of reagents comprise nucleotides of different canonical types.
194. The method of embodiment 192 or 193, wherein said nucleotide solutions comprise labeled nucleotides.
195. The method of any one of embodiments 190-194, wherein each of said first set of reagents, said second set of reagents, said third set of reagents, and said fourth set of reagents comprises a cleavage solution.
196. The method of any one of embodiments 180-195, wherein said first sequencing cycle comprises detecting signal associated with said first nucleic acid sample and said second sequencing cycle comprises detecting signal associated with said second nucleic acid sample.
197. The method of embodiment 196, wherein said signal is fluorescent signal.
198. A system, comprising:
   a sample station comprising a plurality of sample sources comprising a plurality of sample analytes, wherein said plurality of sample analytes comprises a first set of one or more sample analytes, wherein each of said plurality of sample sources is accessible for introduction of sample analytes from said plurality of sample sources into said processing station by one or more actuators;
   a processing station configured to receive sample analytes of said plurality of sample analytes; and
   one or more processors, individually or collectively, programmed to:
      (1) execute at least a portion of a first queuing instruction to introduce said first set of one or more sample analytes from said sample station into said processing station according to a first order of introduction defined by said first queuing instruction, wherein said first queuing instruction defines said first order of introduction of said sample analytes between said plurality of sample sources;
      (2) receive a second queuing instruction, wherein said second queuing instruction defines a second order of introduction different from said first order of introduction; and
      (3) execute at least a portion of said second queuing instruction to introduce a second set of one or more sample analytes of said plurality of sample analytes from said sample station to said processing station according to said second order of introduction while said system is in operation.
199. The system of embodiment 198, wherein said one or more processors are individually or collectively programmed to execute said at least said portion of said second queuing instruction while said processing station is in operation.
200. The system of embodiment 198 or 199, wherein (3) is performed in absence of terminating said operation of said processing station.
201. The system of any one of embodiments 198-200, wherein, during said operation, said processing station is maintained at (i) a different environment than an ambient environment, (ii) a different environment than an environment of said sample station, (iii) a different temperature than an ambient temperature, and/or (iv) a different humidity than an ambient humidity.
202. The system of any one of embodiments 198-201, wherein said processing station is configured to direct a sample analyte from a sample source in said sample station onto a substrate in said processing station.
203. The system of embodiment 202, wherein said substrate is capable of processing a plurality of samples.
204. The system of embodiment 203, wherein a group of samples are selected according to a sample selection instruction based at least in part on use of area of said substrate.
205. The system of embodiment 203, wherein a group of samples are selected such that said group of samples can be processed using a first set of conditions which differs from a second set of conditions at which said other samples are processed.
206. The system of any one of embodiments 198-205, wherein said processing station is configured to direct a reagent to contact a sample analyte from a sample source in said sample station.
207. The system of any one of embodiments 198-206, wherein said processing station is configured to detect a signal associated with a sample analyte from a sample source in said sample station.
208. The system of any one of embodiments 198-207, wherein said processors are individually or collectively programmed to provide a new sample source in said sample station while said system is in operation.
209. The system of any one of embodiments 198-208, wherein said plurality of sample analytes comprises a plurality of nucleic acid molecules.
210. The system of any one of embodiments 198-209, wherein said one or more processors are configured to, within at most 40 hours of running time of said processing station, output one or more selected from the group consisting of:
   (i) at least 1.5 giga reads per substrate,
   (ii) sequence reads averaging at least 140 base pairs (bp) in length, and
   (iii) at least 0.2 terabase reads per run.
211. The system of embodiment 210, wherein said one or more processors are configured to, within at most 40 hours of running time of said processing station, output at least 2.0 giga reads per substrate.
212. The system of embodiment 211, wherein said one or more processors are configured to, within at most 40 hours of running time of said processing station, output at least 6.0 giga reads per substrate.
213. The system of embodiment 212, wherein said one or more processors are configured to, within at most 40 hours of running time of said processing station, output at least 10.0 giga reads per substrate.
214. The system of embodiment 213, wherein said one or more processors are configured to, within at most 40 hours of running time of said processing station, output at least 40.0 giga reads per substrate.
215. The system of any one of embodiments 198-214, wherein said one or more processors are configured to, within at most 40 hours of running time of said processing station, output at least 150 bp read length.
216. The system of embodiment 215, wherein said one or more processors are configured to, within at most 40 hours of running time of said processing station, output at least 250 bp read length.
217. The system of embodiment 216, wherein said one or more processors are configured to, within at most 40 hours of running time of said processing station, output at least 300 bp read length.
218. The system of embodiment 217, wherein said one or more processors are configured to, within at most 40 hours of running time of said processing station, output at least 500 bp read length.
219. The system of any one of embodiments 198-218, wherein said one or more processors are configured to, within at most 40 hours of running time of said processing station, output at least 0.4 terabase reads per run.
220. The system of embodiment 219, wherein said one or more processors are configured to, within at most 40 hours of running time of said processing station, output at least 1.5 terabase reads per run.
221. The system of embodiment 220, wherein said one or more processors are configured to, within at most 40 hours of running time of said processing station, output at least 6.0 terabase reads per run.
222. The system of embodiment 221, wherein said one or more processors are configured to, within at most 40 hours of running time of said processing station, output at least 6.5 terabase reads per run.
223. The system of any one of embodiments 198-210, wherein said one or more processors are configured to, within at most 25 hours of running time of said processing station, output one or more selected from the group consisting of:
   (i) at least 1.5 giga reads per substrate,
   (ii) sequence reads averaging at least 140 base pairs (bp) in length, and
   (iii) at least 0.2 terabase reads per run.
224. The system of any one of embodiments 198-210, wherein said one or more processors are configured to, within at most 15 hours of running time of said processing station, output one or more selected from the group consisting of:
   (i) at least 1.5 giga reads per substrate,
   (ii) sequence reads averaging at least 140 base pairs (bp) in length, and
   (iii) at least 0.2 terabase reads per run.
225. A system comprising:
   a reagent station comprising a first reagent source and a second reagent source, wherein each of said first reagent source and said second reagent source (i) comprises a first reagent and (ii) is accessible for introduction of said first reagent from said reagent station to a processing station by a controller;
   said processing station, wherein said processing station is configured to facilitate one or more operations using said first reagent; and
   one or more processors, individually or collectively, programmed to:
      (1) direct said first reagent from said first reagent source to said processing station;
      (2) direct said first reagent from said second reagent source to said processing station;
      (3) while said processing station is in operation and receiving said first reagent from said second reagent source, (i) replace said first reagent source with a third reagent source comprising said first reagent, wherein said third reagent source is accessible for introduction of said first reagent from said reagent station to said processing station by said controller, or (ii) replenish said first reagent source with an additional volume of said first reagent; and
      (4) direct said first reagent from (i) said third reagent source, or (ii) said additional volume of said first reagent in said first reagent source, to said processing station.
226. The system of embodiment 225, wherein said one or more processors are configured to, within at most 40 hours of running time of said processing station, output one or more selected from the group consisting of:
   (i) at least 1.5 giga reads per substrate,
   (ii) sequence reads averaging at least 140 base pairs (bp) in length, and
   (iii) at least 0.2 terabase reads per run.
227. The system of embodiment 226, wherein said one or more processors are configured to, within at most 40 hours of running time of said processing station, output at least 2.0 Giga reads per substrate.
228. The system of embodiment 227, wherein said one or more processors are configured to, within at most 40 hours of running time of said processing station, output at least 6.0 Giga reads per substrate.
229. The system of embodiment 228, wherein said one or more processors are configured to, within at most 40 hours of running time of said processing station, output at least 10.0 Giga reads per substrate.
230. The system of embodiment 229, wherein said one or more processors are configured to, within at most 40 hours of running time of said processing station, output at least 40.0 Giga reads per substrate.
231. The system of any one of embodiments 225-230, wherein said one or more processors are configured to, within at most 40 hours of running time of said processing station, output at least 150 bp read length.
232. The system of embodiment 231, wherein said one or more processors are configured to, within at most 40 hours of running time of said processing station, output at least 250 bp read length.
233. The system of embodiment 232, wherein said one or more processors are configured to, within at most 40 hours of running time of said processing station, output at least 300 bp read length.
234. The system of embodiment 233, wherein said one or more processors are configured to, within at most 40 hours of running time of said processing station, output at least 500 bp read length.
235. The system of any one of embodiments 225-234, wherein said one or more processors are configured to, within at most 40 hours of running time of said processing station, output at least 0.4 terabase reads per run.
236. The system of embodiment 235, wherein said one or more processors are configured to, within at most 40 hours of running time of said processing station, output at least 1.5 terabase reads per run.
237. The system of embodiment 236, wherein said one or more processors are configured to, within at most 40 hours of running time of said processing station, output at least 6.0 terabase reads per run.
238. The system of embodiment 237, wherein said one or more processors are configured to, within at most 40 hours of running time of said processing station, output at least 6.5 terabase reads per run.
239. The system of any one of embodiments 225-238, wherein said one or more processors are configured to, within at most 25 hours of running time of said processing station, output one or more selected from the group consisting of:
   (i) at least 1.5 giga reads per substrate,
   (ii) sequence reads averaging at least 140 base pairs (bp) in length, and
   (iii) at least 0.2 terabase reads per run.
240. The system of any one of embodiments 225-239, wherein said one or more processors are configured to, within at most 15 hours of running time of said processing station, output one or more selected from the group consisting of:
   (i) at least 1.5 giga reads per substrate,
   (ii) sequence reads averaging at least 140 base pairs (bp) in length, and
   (iii) at least 0.2 terabase reads per run.
241. A system comprising:
   a substrate station comprising a plurality of substrates, wherein each of said plurality of substrates is accessible for introduction of substrates from said substrate station into a processing station by one or more actuators;
   said processing station; and
   one or more processors, individually or collectively, programmed to:
   (1) deliver, by one or more actuators, a first substrate of said plurality of substrates into said processing station;
   (2) in said processing station, perform a process involving an analyte immobilized adjacent to said first substrate; and
   (3) deliver, by said one or more actuators, a second substrate of said plurality of substrates into said processing station while said system is in operation.
242. The system of embodiment 241, wherein said one or more processors are individually or collectively programmed to deliver said second substrate while said processing station is performing said process.
243. The system of embodiment 241 or 242, wherein said one or more processors are configured to, within at most 40 hours of running time of said processing station, output one or more selected from the group consisting of:
   (i) at least 1.5 giga reads per substrate,
   (ii) sequence reads averaging at least 140 base pairs (bp) in length, and
   (iii) at least 0.2 terabase reads per run.
244. The system of embodiment 243 wherein said one or more processors are configured to, within at most 40 hours of running time of said processing station, output at least 2.0 Giga reads per substrate.
245. The system of embodiment 244, wherein said one or more processors are configured to, within at most 40 hours of running time of said processing station, output at least 6.0 Giga reads per substrate.
246. The system of embodiment 245, wherein said one or more processors are configured to, within at most 40 hours of running time of said processing station, output at least 10.0 Giga reads per substrate.
247. The system of embodiment 246, wherein said one or more processors are configured to, within at most 40 hours of running time of said processing station, output at least 40.0 Giga reads per substrate.
248. The system of any one of embodiments 241-247, wherein said one or more processors are configured to, within at most 40 hours of running time of said processing station, output at least 150 bp read length.
249. The system of embodiment 248, wherein said one or more processors are configured to, within at most 40 hours of running time of said processing station, output at least 250 bp read length.
250. The system of embodiment 249, wherein said one or more processors are configured to, within at most 40 hours of running time of said processing station, output at least 300 bp read length.
251. The system of embodiment 250, wherein said one or more processors are configured to, within at most 40 hours of running time of said processing station, output at least 500 bp read length.
252. The system of any one of embodiments 241-251, wherein said one or more processors are configured to, within at most 40 hours of running time of said processing station, output at least 0.4 terabase reads per run.
253. The system of embodiment 252, wherein said one or more processors are configured to, within at most 40 hours of running time of said processing station, output at least 1.5 terabase reads per run.
254. The system of embodiment 253, wherein said one or more processors are configured to, within at most 40 hours of running time of said processing station, output at least 6.0 terabase reads per run.
255. The system of embodiment 254, wherein said one or more processors are configured to, within at most 40 hours of running time of said processing station, output at least 6.5 terabase reads per run.
256. The system of any one of embodiments 241-255, wherein said one or more processors are configured to, within at most 25 hours of running time of said processing station, output one or more selected from the group consisting of:
   (i) at least 1.5 giga reads per substrate,
   (ii) sequence reads averaging at least 140 base pairs (bp) in length, and
   (iii) at least 0.2 terabase reads per run.
257. The system of any one of embodiments 241-255, wherein said one or more processors are configured to, within at most 15 hours of running time of said processing station, output one or more selected from the group consisting of:
   (i) at least 1.5 giga reads per substrate,
   (ii) sequence reads averaging at least 140 base pairs (bp) in length, and
   (iii) at least 0.2 terabase reads per run.
258. A system comprising:
   a processing station configured to receive nucleic acid samples of a plurality of nucleic acid samples from different sample sources and substrates of a plurality of substrates; and
   one or more processors, individually or collectively, programmed to:
   (1) provide a first nucleic acid sample of said plurality of nucleic acid samples to a first substrate of said plurality of substrates;
   (2) provide a second nucleic acid sample of said plurality of nucleic acid samples to a second substrate of said plurality of substrates;
   (3) receive user instructions to start two or more sequencing cycles;
   (4) initiate a first sequencing cycle to process said first nucleic acid sample; and
   (5) during or subsequent to said first sequencing cycle, initiate a second sequencing cycle to process said second nucleic acid sample, wherein said second sequencing cycle is configured to be performed in absence of additional user intervention.
259. The system of embodiment 258, wherein said one or more processors are individually or collectively programmed to, during or subsequent to an (n−1)th sequencing cycle, initiate an nth sequencing cycle to process an nth nucleic acid sample of said plurality of nucleic acid samples on an nth substrate of said plurality of substrates, wherein said nth sequencing cycle is configured to be performed in absence of additional user instructions from said user instructions.
260. The system of embodiment 258 or 259, wherein said one or more processors are configured to, within at most 40 hours of running time of said processing station, output one or more selected from the group consisting of:
   (i) at least 1.5 giga reads per substrate,
   (ii) sequence reads averaging at least 140 base pairs (bp) in length, and
   (iii) at least 0.2 terabase reads per run.
261. The system of embodiment 260, wherein said one or more processors are configured to, within at most 40 hours of running time of said processing station, output at least 2.0 Giga reads per substrate.
262. The system of embodiment 261, wherein said one or more processors are configured to, within at most 40 hours of running time of said processing station, output at least 6.0 Giga reads per substrate.
263. The system of embodiment 262, wherein said one or more processors are configured to, within at most 40 hours of running time of said processing station, output at least 10.0 Giga reads per substrate.
264. The system of embodiment 263, wherein said one or more processors are configured to, within at most 40 hours of running time of said processing station, output at least 40.0 Giga reads per substrate.
265. The system of any one of embodiments 258-264, wherein said one or more processors are configured to, within at most 40 hours of running time of said processing station, output at least 150 bp read length.
266. The system of embodiment 265, wherein said one or more processors are configured to, within at most 40 hours of running time of said processing station, output at least 250 bp read length.
267. The system of embodiment 266, wherein said one or more processors are configured to, within at most 40 hours of running time of said processing station, output at least 300 bp read length.
268. The system of embodiment 267, wherein said one or more processors are configured to, within at most 40 hours of running time of said processing station, output at least 500 bp read length.
269. The system of any one of embodiments 258-268, wherein said one or more processors are configured to, within at most 40 hours of running time of said processing station, output at least 0.4 terabase reads per run.
270. The system of embodiment 269, wherein said one or more processors are configured to, within at most 40 hours of running time of said processing station, output at least 1.5 terabase reads per run.
271. The system of embodiment 270, wherein said one or more processors are configured to, within at most 40 hours of running time of said processing station, output at least 6.0 terabase reads per run.
272. The system of embodiment 271, wherein said one or more processors are configured to, within at most 40 hours of running time of said processing station, output at least 6.5 terabase reads per run.
273. The system of any one of embodiments 258-272, wherein said one or more processors are configured to, within at most 25 hours of running time of said processing station, output one or more selected from the group consisting of:
   (i) at least 1.5 giga reads per substrate,
   (ii) sequence reads averaging at least 140 base pairs (bp) in length, and
   (iii) at least 0.2 terabase reads per run.
274. The system of any one of embodiments 258-272, wherein said one or more processors are configured to, within at most 15 hours of running time of said processing station, output one or more selected from the group consisting of:
   (i) at least 1.5 giga reads per substrate,
   (ii) sequence reads averaging at least 140 base pairs (bp) in length, and
   (iii) at least 0.2 terabase reads per run.
275. The method of any one of embodiments 1-52, wherein said processing station is configured to detect one or more signals or change thereof from said nucleic acid sample.
276. The method of any one of embodiments 1-52, wherein said nucleic acid sequencer further comprises a detection station configured to detect one or more signals or change thereof from said nucleic acid sample.
277. The method of embodiment 276, wherein in (c), said detection system is in operation to detect said one or more signals or change thereof.
278. The method of any one of embodiments 276-277, wherein said processing system comprises said detection station.
279. The system of any one of embodiments 53-104, wherein said processing station is configured to detect one or more signals or change thereof from said nucleic acid sample.
280. The system of any one of embodiments 53-104, further comprising a detection station configured to detect one or more signals or change thereof from said nucleic acid sample.
281. The system of embodiment 280, wherein said detection system is capable of operating to detect said one or more signals or change thereof during performance of said one or more actions.
282. The method of any one of embodiments 280-281, wherein said processing system comprises said detection station.
283. The method of any one of embodiments 105-120, wherein said processing station is configured to detect one or more signals or change thereof from said plurality of sample analytes.
284. The method of any one of embodiments 105-120, wherein said system further comprises a detection station configured to detect one or more signals or change thereof from said plurality of sample analytes.
285. The method of embodiment 284, wherein in (c), said detection system is in operation to detect said one or more signals or change thereof.
286. The method of any one of embodiments 284-285, wherein said processing system comprises said detection station.
287. The method of any one of embodiments 121-149, wherein said processing station is configured to detect one or more signals or change thereof from said analytes.
288. The method of any one of embodiments 121-149, further comprising providing a detection station configured to detect one or more signals or change thereof from said analytes.
289. The method of embodiment 288, wherein in (d), said detection system is in operation to detect said one or more signals or change thereof.
290. The method of any one of embodiments 288-289, wherein said processing system comprises said detection station.
291. The method of any one of embodiments 150-179, wherein said processing station is configured to detect one or more signals or change thereof from said analytes.
292. The method of any one of embodiments 150-179, wherein said system further comprises a detection station configured to detect one or more signals or change thereof from said analytes.
293. The method of embodiment 292, wherein in (d), said detection system is in operation to detect said one or more signals or change thereof.
294. The method of any one of embodiments 292-293, wherein said processing system comprises said detection station.
295. The system of any one of embodiments 198-224, wherein said processing station is configured to detect one or more signals or change thereof from said plurality of sample analytes.
296. The system of any one of embodiments 198-224, further comprising a detection station configured to detect one or more signals or change thereof from said plurality of sample analytes.
297. The system of embodiment 296, wherein said processing system comprises said detection station.
298. The system of any one of embodiments 225-240, wherein said processing station is configured to detect one or more signals or change thereof from a plurality of analytes.
299. The system of any one of embodiments 225-240, further comprising a detection station configured to detect one or more signals or change thereof from a plurality of analytes.
300. The system of embodiment 299, wherein said processing system comprises said detection station.
301. The system of any one of embodiments 241-257, wherein said processing station is configured to detect one or more signals or change thereof from said analyte.
302. The system of any one of embodiments 241-257, further comprising a detection station configured to detect one or more signals or change thereof from said analyte.
303. The system of embodiment 302, wherein said processing system comprises said detection station.
304. The system of any one of embodiments 258-274, wherein said processing station is configured to detect one or more signals or change thereof from said plurality of nucleic acid samples.
305. The system of any one of embodiments 258-274, further comprising a detection station configured to detect one or more signals or change thereof from said plurality of nucleic acid samples.
306. The system of embodiment 305, wherein said processing system comprises said detection station.
307. The method of any one of embodiments 1-52, wherein said nucleic acid sequencer comprises a network of sensors in operative communication with said one or more processors, wherein said one or more processors are configured to, based on one or more signals received from said network of sensors, calibrate, adjust, or maintain a component or process of said processing station, said sample station, said substrate station, or said reagent station, wherein said network of sensors comprises one or more sensors selected from the group consisting of a temperature sensor, pressure sensor, humidity sensor, weight sensor, friction sensor, flow meter, motion sensor, optical sensor, pH sensor, audio sensor, and voltage, current, and/or resistive sensor.
308. The system of any one of embodiments 53-104, further comprising a network of sensors in operative communication with said one or more processors, wherein said one or more processors are configured to, based on one or more signals received from said network of sensors, calibrate, adjust, or maintain a component or process of said processing station, said sample station, said substrate station, or said reagent station, wherein said network of sensors comprises one or more sensors selected from the group consisting of a temperature sensor, pressure sensor, humidity sensor, weight sensor, friction sensor, flow meter, motion sensor, optical sensor, pH sensor, audio sensor, and voltage, current, and/or resistive sensor.

1.-100. (canceled)

101. A method for sequencing a plurality of nucleic acid samples, the method comprising:
(a) providing a nucleic acid sequencer comprising (i) a processing station configured to bring a nucleic acid molecule of a nucleic acid sample of said plurality of nucleic acid samples immobilized adjacent to a substrate into contact with a reagent to sequence said nucleic acid molecule; (ii) a sample station configured to supply said nucleic acid sample to said processing station; (iii) a substrate station configured to supply said substrate to said processing station, which substrate immobilizes adjacent thereto said nucleic acid sample; and (iv) a reagent station configured to supply said reagent to said processing station, wherein said reagent is supplied from a first reservoir or a second reservoir;
(b) executing, by one or more processors individually or collectively, (i) at least a portion of a first queuing instruction to introduce a first set of one or more nucleic acid samples of said plurality of nucleic acid samples, including said nucleic acid sample, from said sample station to said processing station according to a first order of introduction defined by said first queuing instruction; (ii) a substrate loading instruction to introduce said substrate from said substrate station to said processing station and immobilize said first set of one or more nucleic acid samples adjacent to said substrate; and (iii) a sequencing instruction to draw said reagent from said first reservoir, from said second reservoir, or alternately from said first reservoir and said second reservoir and deliver said reagent to said processing station; and
(c) while said processing station is in operation, performing one or more actions selected from the group consisting of:
(1) introducing an additional nucleic acid sample to said sample station,
(2) inputting a second queuing instruction and executing at least a portion of said second queuing instruction, wherein said second queuing instruction defines a second order of introduction that is different than said first order of introduction,
(3) introducing an additional substrate to said substrate station, and
(4) introducing an additional volume of said reagent to said reagent station by one or more of (i) replacing said first reservoir or said second reservoir with a third reservoir containing said reagent and (ii) replenishing said first reservoir or said second reservoir with said reagent.

102. The method of claim 101, wherein said processing station is configured to operate for at least 12 hours without human intervention.

103. The method of claim 101, wherein (c) comprises performing two or more actions selected from the group consisting of (1), (2), (3), and (4).

104. The method of claim 101, wherein said sequencing instruction in (b)(iii) comprises instructions to draw said reagent from said first reservoir until said first reservoir is depleted below a predetermined threshold, then to draw said reagent from said second reservoir.

105. The method of claim 101, wherein (4) comprises replacing or replenishing a reservoir from said first reservoir and said second reservoir that is depleted below a predetermined threshold.

106. The method of claim 101, wherein said reagent comprises one or more members selected from the group consisting of a nucleotide solution, a cleaving solution, and a washing solution.

107. The method of claim 106, wherein said nucleotide solution comprises one or more members selected from the group consisting of adenine-containing nucleotides, cytosine-containing nucleotides, guanine-containing nucleotides, thymine-containing nucleotides, and uracil-containing nucleotides.

108. The method of claim 106, wherein said nucleotide solution comprises labeled nucleotides.

109. The method of claim 101, wherein said substrate comprises a substantially planar array.

110. The method of claim 101, wherein said substrate comprises a plurality of independently addressable locations.

111. The method of claim 101, wherein said substrate is configured to rotate about an axis in said processing station.

112. The method of claim 101, wherein said nucleic acid molecule is coupled to a bead, wherein said bead is immobilized adjacent to said substrate.

113. The method of claim 101, wherein said plurality of nucleic acid samples is immobilized adjacent to said substrate, wherein nucleic acid samples of said plurality of nucleic acid samples are from different sources.

114. The method of claim 101, wherein said processing station is disposed in a first environment different from a second environment in which said sample station, said substrate station, and/or said reagent station is disposed.

115. The method of claim 114, wherein said first environment has a higher relative humidity than said second environment.

116. The method of claim 101, wherein said nucleic acid sequencer comprises a dilution station configured to dilute said reagent from said reagent station prior to delivery of said reagent to said processing station.

117. The method of claim 101, wherein said substrate station comprises a vacuum desiccator.

118. The method of claim 101, wherein said one or more processors are configured to, within at most 25 hours of running time of said processing station, output one or more selected from the group consisting of:
(i) sequence reads averaging at least 250 base pairs (bp) in length, and
(ii) at least 1.5 terabase reads per run.

119. The method of claim 101, further comprising (A) inputting (1) said plurality of nucleic acid samples, including said nucleic acid sample, to said sample station and (2) a plurality of substrates, including said substrate, to said substrate station; and (B) providing to said one or more processors user instructions to start two or more sequencing cycles.

120. The method of claim 101, wherein said nucleic acid sequencer comprises a network of sensors in operative communication with said one or more processors, wherein said one or more processors are configured to, based on one or more signals received from said network of sensors, calibrate, adjust, or maintain a component or process of said processing station, said sample station, said substrate station, or said reagent station, wherein said network of sensors comprises one or more sensors selected from the group consisting of a temperature sensor, pressure sensor, humidity sensor, weight sensor, friction sensor, flow meter, motion sensor, optical sensor, pH sensor, audio sensor, and voltage, current, or resistive sensor.
